(19) United States
(12) Patent Application Publication
(10) Pub. No.: US 2023/0212628 A1
(43) Pub. Date: Jul. 6, 2023

(54) Production of Sialylated Oligosaccharide in Host Cells

(71) Applicant: Inbiose N.V., Zwijnaarde (BE)

(72) Inventors: Joeri Beauprez, Zwijnaarde (BE); Pieter Coussement, Zwijnaarde (BE); Thomas Decoene, Zwijnaarde (BE); Annelies Vercauteren, Zwijnaarde (BE)

(21) Appl. No.: 17/787,115

(22) PCT Filed: Dec. 18, 2020
(86) PCT No.: PCT/EP2020/086950

§ 371 Date: Jun. 17, 2022

(30) Foreign Application Priority Data
Dec. 18, 2019 (BE) BE2019/5935
Dec. 18, 2019 (BE) BE2019/5936
Dec. 18, 2019 (BE) BE2019/5937
Dec. 18, 2019 (BE) BE2019/5938
Dec. 18, 2019 (BE) BE2019/5939

(51) Int. Cl. C12P 19/12; C12N 9/10; C12N 15/70; C12P 19/18

(52) U.S. Cl. CPC C12P 19/12; C12N 9/1081; C12Y 204/99001; C12N 15/70; C12P 19/18; C12N 2800/101

(57) ABSTRACT

The present invention is in the technical field of synthetic biology and metabolic engineering. More particularly, the present invention is in the technical field of fermentation of metabolically engineered host cells. The present invention describes a method of making sialylated oligosaccharide by fermentation with a genetically modified cell, as well as to the genetically modified cell used in the method. The genetically modified cell comprises at least one nucleic acid sequence coding for an enzyme involved in sialylated oligosaccharide synthesis and at least one nucleic acid expressing a membrane protein.

### FIELD OF THE INVENTION

The present invention is in the technical field of synthetic biology and metabolic engineering. More particularly, the present invention is in the technical field of fermentation of metabolically engineered host cells. The present invention describes a method of producing sialylated oligosaccharides by fermentation with a genetically modified cell, as well as to the genetically modified cell used in the method. The genetically modified cell comprises at least one nucleic acid sequence coding for an enzyme involved in sialylated oligosaccharide synthesis and at least one nucleic acid expressing a membrane protein.

### BACKGROUND

Today, more than 80 compounds belonging to the family of Human Milk Oligosaccharides (HMOs), have been structurally characterized. These HMOs represent a class of complex oligosaccharides that function as prebiotics. Additionally, the structural homology of HMO to epithelial epitopes accounts for protective properties against bacterial pathogens. Within the infant gastrointestinal tract, HMOs selectively nourish the growth of selected bacterial strains and are, thus, priming the development of a unique gut microbiota in breast milk-fed infants.

Some of these Human Milk oligosaccharides require the presence of particular sialylated structures which most likely exhibit a particular biological activity. Production of these sialylated oligosaccharides requires the action of a sialyltransferase. Such sialyltransferases, which belong to enzyme family of glycosyltransferases, are widely expressed in vertebrates, invertebrates, plants, fungi, yeasts and bacteria. They catalyze the transfer of a sialic acid to an acceptor, which include di- and oligosaccharides, (glyco)proteins and (glyco)lipids. The thus sialylated acceptor substrates are involved in a variety of biological and pathological processes.

In microbial fermentative production of sialylated oligosaccharides, the sialylated oligosaccharide is in many cases produced intracellularly in the industrial production host. One problem identified in the art as the true difficulty in producing oligosaccharides in cells is the intracellular enrichment of the produced oligosaccharides and their extraction. The intracellular enrichment is deemed to be responsible for the product-inhibitory effect on the production of the desired oligosaccharide. Synthesis may become slow or the desired oligosaccharide may reach cytotoxic concentrations resulting in metabolic arrest or even cell lysis.

It is an object of the present invention to provide for tools and methods by means of which sialylated oligosaccharides can be produced in an efficient, time and cost-effective way and which yield high amounts of the desired product.

According to the invention, this and other objects are achieved by providing a method and a cell for the production of sialylated oligosaccharide wherein the cell is genetically modified for the production of said sialylated oligosaccharide and comprises at least one nucleic acid sequence encoding an enzyme involved in sialylated oligosaccharide synthesis. The cell furthermore also expresses a membrane protein according to the present invention.

### DESCRIPTION

### Summary of the Invention

Surprisingly it has now been found that the membrane proteins used in the present invention provide for newly identified membrane proteins having a positive effect on fermentative production of sialylated oligosaccharide, providing a better yield, productivity, specific productivity and/or growth speed when used to genetically engineer a host cell producing sialylated oligosaccharide.

The invention also provides methods for producing sialylated oligosaccharide. The sialylated oligosaccharide is obtained with a host cell comprising the membrane protein of the present invention.

### Definitions

The words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself.

The various embodiments and aspects of embodiments of the invention disclosed herein are to be understood not only in the order and context specifically described in this specification, but to include any order and any combination thereof. Whenever the context requires, all words used in the singular number shall be deemed to include the plural and vice versa. Unless defined otherwise, all technical and scientific terms used herein generally have the same meaning as commonly understood by one of ordinary skill in the art to which this invention belongs. Generally, the nomenclature used herein and the laboratory procedures in cell culture, molecular genetics, organic chemistry and nucleic acid chemistry and hybridization described herein are those well-known and commonly employed in the art. Standard techniques are used for nucleic acid and peptide synthesis. Generally, enzymatic reactions and purification steps are performed according to the manufacturer's specifications.

In the drawings and specification, there have been disclosed embodiments of the invention, and although specific terms are employed, the terms are used in a descriptive sense only and not for purposes of limitation, the scope of the invention being set forth in the following claims. It must be understood that the illustrated embodiments have been set forth only for the purposes of example and that it should not be taken as limiting the invention. It will be apparent to those skilled in the art that alterations, other embodiments, improvements, details and uses can be made consistent with the letter and spirit of the disclosure herein and within the scope of this disclosure, which is limited only by the claims, construed in accordance with the patent law, including the doctrine of equivalents. In the claims which follow, reference characters used to designate claim steps are provided for convenience of description only, and are not intended to imply any particular order for performing the steps.

According to the present invention, the term “polynucleotide(s)” generally refers to any polyribonucleotide or polydeoxyribonucleotide, which may be unmodified RNA or DNA or modified RNA or DNA. “Polynucleotide(s)” include, without limitation, single- and double-stranded DNA, DNA that is a mixture of single- and double-stranded regions or single-, double- and triple-stranded regions, single- and double-stranded RNA, and RNA that is mixture of single- and double-stranded regions, hybrid molecules comprising DNA and RNA that may be single-stranded or, more typically, double-stranded, or triple-stranded regions, or a mixture of single- and double-stranded regions. In addition, “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. As used herein, the term “polynucleotide(s)” also includes DNAs or RNAs as described above that contain one or more modified bases. Thus, DNAs or RNAs with backbones modified for stability or for other reasons are “polynucleotide(s)” according to the present invention. Moreover, DNAs or RNAs comprising unusual bases, such as inosine, or modified bases, such as tritylated bases, are to be understood to be covered by the term “polynucleotides”. It will be appreciated that a great variety of modifications have been made to DNA and RNA that serve many useful purposes known to those of skill in the art. The term “polynucleotide(s)” as it is employed herein embraces such chemically, enzymatically or metabolically modified forms of polynucleotides, as well as the chemical forms of DNA and RNA characteristic of viruses and cells, including, for example, simple and complex cells. The term “polynucleotide(s)” also embraces short polynucleotides often referred to as oligonucleotide(s).

“Polypeptide(s)” refers to any peptide or protein comprising two or more amino acids joined to each other by peptide bonds or modified peptide bonds. “Polypeptide(s)” refers to both short chains, commonly referred to as peptides, oligopeptides and oligomers and to longer chains generally referred to as proteins. Polypeptides may contain amino acids other than the 20 gene encoded amino acids. “Polypeptide(s)” include those modified either by natural processes, such as processing and other post-translational modifications, but also by chemical modification techniques. Such modifications are well described in basic texts and in more detailed monographs, as well as in a voluminous research literature, and they are well known to the skilled person. The same type of modification may be present in the same or varying degree at several sites in a given polypeptide. Furthermore, a given polypeptide may contain many types of modifications. Modifications can occur anywhere in a polypeptide, including the peptide backbone, the amino acid side-chains, and the amino or carboxyl termini. Modifications include, for example, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, gamma-carboxylation of glutamic acid residues, hydroxylation and ADP-ribosylation, selenoylation, transfer-RNA mediated addition of amino acids to proteins, such as arginylation, and ubiquitination. Polypeptides may be branched or cyclic, with or without branching. Cyclic, branched and branched circular polypeptides may result from post-translational natural processes and may be made by entirely synthetic methods, as well.

“Isolated” means altered “by the hand of man” from its natural state, i.e., if it occurs in nature, it has been changed or removed from its original environment, or both. For example, a polynucleotide or a polypeptide naturally present in a living organism is not “isolated,” but the same polynucleotide or polypeptide separated from the coexisting materials of its natural state is “isolated”, as the term is employed herein. Similarly, a “synthetic” sequence, as the term is used herein, means any sequence that has been generated synthetically and not directly isolated from a natural source. “Synthesized”, as the term is used herein, means any synthetically generated sequence and not directly isolated from a natural source.

“Recombinant” means genetically engineered DNA prepared by transplanting or splicing genes from one species into the cells of a host organism of a different species. Such DNA becomes part of the host's genetic makeup and is replicated. “Mutant” cell or microorganism as used within the context of the present disclosure refers to a cell or microorganism which is genetically engineered or has an altered genetic make-up.

The term “endogenous,” within the context of the present disclosure refers to any polynucleotide, polypeptide or protein sequence which is a natural part of a cell and is occurring at its natural location in the cell chromosome. The term “exogenous” refers to any polynucleotide, polypeptide or protein sequence which originates from outside the cell under study and not a natural part of the cell or which is not occurring at its natural location in the cell chromosome or plasmid.

The term “heterologous” when used in reference to a polynucleotide, gene, nucleic acid, polypeptide, or enzyme refers to a polynucleotide, gene, nucleic acid, polypeptide, or enzyme that is from a source or derived from a source other than the host organism species. In contrast, a “homologous” polynucleotide, gene, nucleic acid, polypeptide, or enzyme is used herein to denote a polynucleotide, gene, nucleic acid, polypeptide, or enzyme that is derived from the host organism. When referring to a gene regulatory sequence or to an auxiliary nucleic acid sequence used for maintaining or manipulating a gene sequence (e.g. a promoter, a 5′ untranslated region, 3′ untranslated region, poly A addition sequence, intron sequence, splice site, ribosome binding site, internal ribosome entry sequence, genome homology region, recombination site, etc.), “heterologous” means that the regulatory sequence or auxiliary sequence is not naturally associated with the gene with which the regulatory or auxiliary nucleic acid sequence is juxtaposed in a construct, genome, chromosome, or episome. Thus, a promoter operably linked to a gene to which it is not operably linked to in its natural state (i.e. in the genome of a non-genetically engineered organism) is referred to herein as a “heterologous promoter,” even though the promoter may be derived from the same species (or, in some cases, the same organism) as the gene to which it is linked.

The term “polynucleotide encoding a polypeptide” as used herein encompasses polynucleotides that include a sequence encoding a polypeptide of the invention. The term also encompasses polynucleotides that include a single continuous region or discontinuous regions encoding the polypeptide (for example, interrupted by integrated phage or an insertion sequence or editing) together with additional regions that also may contain coding and/or non-coding sequences.

The term “modified expression” of a gene relates to a change in expression compared to the wild type expression of said gene in any phase of the production process of the sialylated oligosaccharide. Said modified expression is either a lower or higher expression compared to the wild type, wherein the term “higher expression” is also defined as “overexpression” of said gene in the case of an endogenous gene or “expression” in the case of a heterologous gene that is not present in the wild type strain. Lower expression is obtained by means of common well-known technologies for a skilled person (such as the usage of siRNA, CRISPR, CRISPRi, recombineering, homologous recombination, ssDNA mutagenesis, RNAi, miRNA, asRNA, mutating genes, knocking-out genes, transposon mutagenesis, . . . ) which are used to change the genes in such a way that they are less-able (i.e. statistically significantly ‘less-able’ compared to a functional wild-type gene) or completely unable (such as knocked-out genes) to produce functional final products. Overexpression or expression is obtained by means of common well-known technologies for a skilled person, wherein said gene is part of an “expression cassette” which relates to any sequence in which a promoter sequence, untranslated region sequence (containing either a ribosome binding sequence or Kozak sequence), a coding sequence (for instance a membrane protein gene sequence) and optionally a transcription terminator is present, and leading to the expression of a functional active protein. Said expression is either constitutive or conditional or regulated.

The term “constitutive expression” is defined as expression that is not regulated by transcription factors other than the subunits of RNA polymerase (e.g. the bacterial sigma factors) under certain growth conditions. Non-limiting examples of such transcription factors are CRP, LacI, ArcA, Cra, IcIR in E. coli, or, Aft2p, Crz1p, Skn7 in Saccharomyces cerevisiae, or, DeoR, GntR, Fur in B. subtilis. These transcription factors bind on a specific sequence and may block or enhance expression in certain growth conditions. RNA polymerase binds a specific sequence to initiate transcription, for instance via a sigma factor in prokaryotic hosts.

The term “regulated expression” is defined as expression that is regulated by transcription factors other than the subunits of RNA polymerase (e.g. bacterial sigma factors) under certain growth conditions. Examples of such transcription factors are described above. Commonly expression regulation is obtained by means of an inducer, such as but not limited to IPTG, arabinose, rhamnose, fucose, allo-lactose or pH shifts, or temperature shifts or carbon depletion or substrates or the produced product.

The term “wild type” refers to the commonly known genetic or phenotypical situation as it occurs in nature.

“Variant(s)” as the term is used herein, is a polynucleotide or polypeptide that differs from a reference polynucleotide or polypeptide, respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one encoded by the genetic code. A variant of a polynucleotide or polypeptide may be naturally occurring such as an allelic variant, or it may be a variant that is not known to occur naturally. Non-naturally occurring variants of polynucleotides and polypeptides may be made by mutagenesis techniques, by direct synthesis, and by other recombinant methods known to the persons skilled in the art.

In some embodiments, the present disclosure contemplates making functional variants by modifying the structure of a membrane protein as used in the present invention. Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, and in case of the present invention to provide better yield, productivity, and/or growth speed than a cell without the variant.

The term “functional homolog” as used herein describes those molecules that have sequence similarity and also share at least one functional characteristic such as a biochemical activity. Functional homologs will typically give rise to the same characteristics to a similar, but not necessarily the same, degree. Functionally homologous proteins give the same characteristics where the quantitative measurement produced by one homolog is at least 10 percent of the other; more typically, at least 20 percent, between about 30 percent and about 40 percent; for example, between about 50 percent and about 60 percent; between about 70 percent and about 80 percent; or between about 90 percent and about 95 percent; between about 98 percent and about 100 percent, or greater than 100 percent of that produced by the original molecule. Thus, where the molecule has enzymatic activity the functional homolog will have the above-recited percent enzymatic activities compared to the original enzyme. Where the molecule is a DNA-binding molecule (e.g., a polypeptide) the homolog will have the above-recited percentage of binding affinity as measured by weight of bound molecule compared to the original molecule.

A functional homolog and the reference polypeptide may be naturally occurring polypeptides, and the sequence similarity may be due to convergent or divergent evolutionary events. Functional homologs are sometimes referred to as orthologs, where “ortholog” refers to a homologous gene or protein that is the functional equivalent of the referenced gene or protein in another species.

Functional homologs can be identified by analysis of nucleotide and polypeptide sequence alignments. For example, performing a query on a database of nucleotide or polypeptide sequences can identify homologs of biomass-modulating polypeptides. Sequence analysis can involve BLAST, Reciprocal BLAST, or PSI-BLAST analysis of non-redundant databases using amino acid sequence of a biomass-modulating polypeptide as the reference sequence. Amino acid sequence is, in some instances, deduced from the nucleotide sequence. Typically, those polypeptides in the database that have greater than 40 percent sequence identity are candidates for further evaluation for suitability as a biomass-modulating polypeptide. Amino acid sequence similarity allows for conservative amino acid substitutions, such as substitution of one hydrophobic residue for another or substitution of one polar residue for another. If desired, manual inspection of such candidates can be carried out in order to narrow the number of candidates to be further evaluated. Manual inspection can be performed by selecting those candidates that appear to have domains present in productivity-modulating polypeptides, e.g., conserved functional domains.

“Fragment”, with respect to a polynucleotide, refers to a clone or any part of a polynucleotide molecule, particularly a part of a polynucleotide that retains a usable, functional characteristic. Useful fragments include oligonucleotides and polynucleotides that may be used in hybridization or amplification technologies or in the regulation of replication, transcription or translation. A “polynucleotide fragment” refers to any subsequence of a polynucleotide, typically, of at least about 9 consecutive nucleotides, for example at least about 30 nucleotides or at least about 50 nucleotides of any of the sequences provided herein. Exemplary fragments can additionally or alternatively include fragments that comprise, consist essentially of, or consist of a region that encodes a conserved family domain of a polypeptide. Exemplary fragments can additionally or alternatively include fragments that comprise a conserved domain of a polypeptide.

Fragments may additionally or alternatively include subsequences of polypeptides and protein molecules, or a subsequence of the polypeptide. In some cases, the fragment or domain is a subsequence of the polypeptide which performs at least one biological function of the intact polypeptide in substantially the same manner, or to a similar extent, as does the intact polypeptide. For example, a polypeptide fragment can comprise a recognizable structural motif or functional domain such as a DNA-binding site or domain that binds to a DNA promoter region, an activation domain, or a domain for protein-protein interactions, and may initiate transcription. Fragments can vary in size from as few as 3 amino acid residues to the full length of the intact polypeptide, for example at least about 20 amino acid residues in length, for example at least about 30 amino acid residues in length. Preferentially a fragment is a functional fragment that has at least one property or activity of the polypeptide from which it is derived, such as, for example, the fragment can include a functional domain or conserved domain of a polypeptide. A domain can be characterized, for example, by a Pfam or Conserved Domain Database (CDD) designation.

The term “sialylated oligosaccharide” as used herein refers to a sugar polymer containing at least two monosaccharide units, at least one of which is a sialyl (N-acetylneuraminyl) moiety. The sialylated oligosaccharide can have a linear or branched structure containing monosaccharide units that are linked to each other by interglycosidic linkage.

As used herein, a ‘sialylated oligosaccharide’ is furthermore to be understood as a charged sialic acid containing oligosaccharide, i.e. an oligosaccharide having a sialic acid residue. It has an acidic nature. Some examples are 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Aca-2,3Gal beta-1,3GalNAc beta-1,3Gala-1,4Gal beta-1,4Gal), sialylated tetrasaccharide (Neu5Aca-2,3Gal beta-1,4GlcNAc beta-14GlcNAc), pentasaccharide LSTD (Neu5Aca-2,3Gal beta-1,4GlcNAc beta-1,3Gal beta-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s), including but not limited to: oligosaccharide moieties of the gangliosides selected from GM3 (3′ sialyllactose, Neu5Acα-2,3Gal β-4Glc) and oligosaccharides comprising the GM3 motif, GD3 (Neu5Acα-2,8Neu5Acα-2,3Gal β-1,4Glc), GT3 (Neu5Acα-2,8Neu5Acα-2,8Neu5Acα-2,3Gal β-1,4Glc), GM2 (GalNAc β-1,4(Neu5Acα-2,3)Gal β-1,4Glc), GM1 (Gal β-1,3GalNAc β-1,4(Neu5Acα-2,3)Gal β-1,4Glc), GD1a (Neu5Acα-2,3Gal β-1,3GalNAc β-1,4(Neu5Acα-2,3)Gal β-1,4Glc), GT1a (Neu5Acα-2,8Neu5Acα-2,3Gal β-1,3GalNAc β-1,4(Neu5Acα-2,3)Gal β-1,4Glc), GD2 (GalNAc β-1,4(Neu5Acα-2,8Neu5Acα2,3)Gal β-1,4Glc), GT2 (GalNAc β-1,4(Neu5Acα-2,8Neu5Acα-2,8Neu5Acα2,3)Gal β-1,4Glc), GD1b (Gal β-1,3GalNAc β-1,4(Neu5Acα-2,8Neu5Acα2,3)Gal β-1,4Glc), GT1b (Neu5Acα-2,3Gal β-1,3GalNAc β-1,4(Neu5Acα-2,8Neu5Acα2,3)Gal β-1,4Glc), GQ1b (Neu5Acα-2,8Neu5Acα-2,3Gal β-1,3GalNAc β-1,4(Neu5Acα-2,8Neu5Acα2,3)Gal β-1,4Glc), GT1c (Gal β-1,3GalNAc β-1,4(Neu5Acα-2,8Neu5Acα-2,8Neu5Acα2,3)Gal β-1,4Glc), GQ1c (Neu5Acα-2,3Gal β-1,3GalNAc β-1,4(Neu5Acα-2,8Neu5Acα-2,8Neu5Acα2,3)Gal β-1,4Glc), GP1c (Neu5Acα-2,8Neu5Acα-2,3Gal β-1,3GalNAc β-1,4(Neu5Acα-2,8Neu5Acα-2,8Neu5Acα2,3)Gal β-1,4Glc), GD1a (Neu5Acα-2,3Gal β-1,3(Neu5Acα-2,6)GalNAc β-1,4Gal β-1,4Glc), Fucosyl-GM1 (Fucα-1,2Gal β-1,3GalNAc β-1,4(Neu5Acα-2,3)Gal β-1,4Glc); all of which may be extended to the production of the corresponding gangliosides by reacting the above oligosaccharide moieties with ceramide or synthetizing the above oligosaccharides on a ceramide.

Preferably the sialylated oligosaccharide is a sialylated mammalian milk oligosaccharide, also known as acidic mammalian milk oligosaccharides. Examples of acidic mammalian milk oligosaccharides include, but are not limited to, 3′-sialyllactose (3′-O-sialyllactose, 3′-SL, 3′SL), 6′-sialyllactose (6′-O-sialyllactose, 6′-SL, 6′SL), 3-fucosyl-3′-sialyllactose (3′-O-sialyl-3-O-fucosyllactose, FSL), 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-LNH (SLNH), sialyl-lacto-N-hexaose (SLNH), sialyl-lacto-N-neohexaose I (SLNH-I), sialyl-lacto-N-neohexaose 11 (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose. In some sialylated mammalian milk oligosaccharides the sialic acid residue is preferably linked to the 3-O- and/or 6-O-position of a terminal D-galactose or to the 6-O-position of a non-terminal GlcNAc residue via α-glycosidic linkages.

The terms “cell genetically modified for the production of sialylated oligosaccharide” within the context of the present disclosure refers to a cell of a microorganism which is genetically manipulated to comprise at least one of i) a gene encoding a sialyltransferase necessary for the synthesis of said sialylated oligosaccharide, ii) a biosynthetic pathway to produce a sialic acid nucleotide donor suitable to be transferred by said sialyltransferase to a carbohydrate precursor, and/or iii) a biosynthetic pathway to produce lactose or a mechanism of internalization of a precursor from the culture medium into the cell where it is sialylated to produce the sialylated oligosaccharide.

The terms “nucleic acid sequence coding for an enzyme for sialylated oligosaccharide synthesis” relates to nucleic acid sequences coding for enzymes necessary in the synthesis pathway to the sialylated oligosaccharide. Examples of such enzymes are fructose-6-P-aminotransferases (e.g. glmS), glucosamine-6-P-aminotransferases (e.g. a heterologous GNA1), (native) phosphatases, N-acetylglucosamine epimerases (e.g. a heterologous AGE), sialic acid synthases (e.g. a heterologous neuB), CMP-sialic acid synthetases (e.g. a heterologous neuA), UDP-N-acetylglucosamine-2-epimerases, ManNAc kinase forming ManNAc-6P, sialic acid phosphate synthetase forming Neu5Ac-9P, sialic acid phosphatase forming sialic acid, sialyltransferases, alfa-2,3-sialyltransferase, alfa-2,6-sialyltransferase, alfa-2,8-sialyltransferase.

“Oligosaccharide” as the term is used herein and as generally understood in the state of the art, refers to a saccharide polymer containing a small number, typically two to ten, of simple sugars, i.e. monosaccharides.

The term “membrane proteins” as used herein refers to proteins that are part of or interact with the cell membrane and control the flow of molecules and information across the cell. The membrane proteins are thus involved in transport, be it import into or export out of the cell.

The major facilitator superfamily (MFS) is a superfamily of membrane transport proteins catalyzing uniport, solute:cation (H+, but seldom Na+) symport and/or solute:H+ or solute:solute antiport. Most are of 400-600 amino acyl residues in length and possess either 12, 14, or occasionally, 24 transmembrane α-helical spanners (TMSs) as defined by the Transporter Classification Database operated by the Saier Lab Bioinformatics Group available via www.tcdb.org and providing a functional and phylogenetic classification of membrane transport proteins.

“SET” or “Sugar Efflux Transporter” as used herein refers to membrane proteins of the SET family which are proteins with InterPRO domain IPR004750 and/or are proteins that belong to the eggNOGv4.5 family ENOG410XTE9. Identification of the InterPro domain can be done by using the online tool on https://www.ebi.ac.uk/interpro/ or a standalone version of InterProScan (https://www.ebi.ac.uk/interpro/download.html) using the default values. Identification of the orthology family in eggNOGv4.5 can be done using the online version or a standalone version of eggNOG-mapperv1 (http://eggnogdb.embl.de/#/app/home). It should be understood for those skilled in the art that for the databases used herein, comprising eggnogdb 4.5.1 (released September 2016) and InterPro 75.0 (released 4 Jul. 2019), the content of each database is fixed at each release and is not to be changed. When the content of a specific database is changed, this specific database receives a new release version with a new release date. All release versions for each database with their corresponding release dates and specific content as annotated at these specific release dates are available and known to those skilled in the art.

The term “Siderophore” as used herein is referring to the secondary metabolite of various microorganisms which are mainly ferric ion specific chelators. These molecules have been classified as catecholate, hydroxamate, carboxylate and mixed types.

Siderophores are in general synthesized by a nonribosomal peptide synthetase (NRPS) dependent pathway or an NRPS independent pathway (NIS). The most important precursor in NRPS-dependent siderophore biosynthetic pathway is chorismate. 2, 3-DHBA could be formed from chorismate by a three-step reaction catalyzed by isochorismate synthase, isochorismatase, and 2, 3-dihydroxybenzoate-2, 3-dehydrogenase. Siderophores can also be formed from salicylate which is formed from isochorismate by isochorismate pyruvate lyase. When ornithine is used as precursor for siderophores, biosynthesis depends on the hydroxylation of ornithine catalysed by L-ornithine N5-monooxygenase. In the NIS pathway, an important step in siderophore biosynthesis is N(6)-hydroxylysine synthase.

A transporter is needed to export the siderophore outside the cell. Four superfamilies of membrane proteins are identified so far in this process: the major facilitator superfamily (MFS); the Multidrug/Oligosaccharidyl-lipid/Polysaccharide Flippase Superfamily (MOP); the resistance, nodulation and cell division superfamily (RND); and the ABC superfamily. In general, the genes involved in siderophore export are clustered together with the siderophore biosynthesis genes. The term “siderophore exporter” as used herein refers to such transporters needed to export the siderophore outside of the cell.

The term “purified” refers to material that is substantially or essentially free from components which interfere with the activity of the biological molecule. For cells, saccharides, nucleic acids, and polypeptides, the term “purified” refers to material that is substantially or essentially free from components which normally accompany the material as found in its native state. Typically, purified saccharides, oligosaccharides, proteins or nucleic acids of the invention are at least about 50%, 55%, 60%, 65%, 70%, 75%, 80% or 85% pure, usually at least about 90%, 91%, 92%, 93%, 94% 95%, 96%, 97%, 98%, or 99% pure as measured by band intensity on a silver stained gel or other method for determining purity. Purity or homogeneity can be indicated by a number of means well known in the art, such as polyacrylamide gel electrophoresis of a protein or nucleic acid sample, followed by visualization upon staining. For certain purposes high resolution will be needed and HPLC or a similar means for purification utilized. For oligosaccharides, e.g., 3-sialyllactose, purity can be determined using methods such as but not limited to thin layer chromatography, gas chromatography, NMR, HPLC, capillary electrophoresis or mass spectroscopy.

The terms “identical” or percent “identity” or % “identity” in the context of two or more nucleic acid or polypeptide sequences, refer to two or more sequences or subsequences that are the same or have a specified percentage of amino acid residues or nucleotides that are the same, when compared and aligned for maximum correspondence, as measured using sequence comparison algorithms or by visual inspection. For sequence comparison, one sequence acts as a reference sequence, to which test sequences are compared. When using a sequence comparison algorithm, test and reference sequences are inputted into a computer, subsequence coordinates are designated, if necessary, and sequence algorithm program parameters are designated. The sequence comparison algorithm then calculates the percent sequence identity for the test sequence(s) relative to the reference sequence, based on the designated program parameters. Percent identity can be determined using BLAST and PSI-BLAST (Altschul et al., 1990, J Mol Biol 215:3, 403-410; Altschul et al., 1997, Nucleic Acids Res 25: 17, 3389-402). For the purposes of this invention, percent identity is determined using MatGAT2.01 (Campanella et al., 2003, BMC Bioinformatics 4:29). The following default parameters for protein are employed: (1) Gap cost Existence: 12 and Extension: 2; (2) The Matrix employed was BLOSUM50.

The term “control sequences” refers to sequences recognized by the host cells transcriptional and translational systems, allowing transcription and translation of a polynucleotide sequence to a polypeptide. Such DNA sequences are thus necessary for the expression of an operably linked coding sequence in a particular host cell or organism. Such control sequences can be, but are not limited to, promoter sequences, ribosome binding sequences, Shine Dalgarno sequences, Kozak sequences, transcription terminator sequences. The control sequences that are suitable for prokaryotes, for example, include a promoter, optionally an operator sequence, and a ribosome binding site. Eukaryotic cells are known to utilize promoters, polyadenylation signals, and enhancers. DNA for a presequence or secretory leader may be operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it affects the transcription of the sequence; or a ribosome binding site is operably linked to a coding sequence if it is positioned so as to facilitate translation. Said control sequences can furthermore be controlled with external chemicals, such as, but not limited to, IPTG, arabinose, lactose, allo-lactose, rhamnose or fucose via an inducible promoter or via a genetic circuit that either induces or represses the transcription or translation of said polynucleotide to a polypeptide.

Generally, “operably linked” means that the DNA sequences being linked are contiguous, and, in the case of a secretory leader, contiguous and in reading phase. However, enhancers do not have to be contiguous.

As used herein, the term “cell productivity index (CPI)” refers to the mass of the product produced by the cells divided by the mass of the cells produced in the culture.

As used herein, the term “whole broth concentration” is defined as the concentration measured by first disrupting the cells via methods known in the art such as but not limited to sonication, homogenization (Cell Lysor, Disperser, High Shear Mixer, Homogenizer, Polytron, Rotor Stator Homogenizer, Sonicator or Tissue Tearor), bead disruption with glass, ceramic, steel, beads, Cryopulverization, High Pressure Cell Disruption, such as but not limited to French press, Nitrogen decompression, enzymatic lysis with enzymes such as but not limited to proteases, glycanases, and/or lysozyme. Second, the liquid is separated from the solids through methods such as but not limited to centrifugation, filtration, flocculation, precipitation. Third, the sialylated oligosaccharide is measured through methods well known in the art such as but not limited to HPLC combined with RI, ELSD, CAD, MS, UV, fluorescence detector, DAD or HPAEC combined with PAD or GC with FID or MS, NMR, TLC, HP-TLC or MALDI TOF.

The term “supernatant concentration” is defined as the concentration measured by first removing the undisrupted cells from the medium, removing the solids from the liquid through methods such as but not limited to centrifugation, filtration, flocculation, precipitation. Also here, the sialylated oligosaccharide is measured through methods well known in the art such as but not limited to HPLC combined with RI, ELSD, CAD, MS, UV, fluorescence detector, DAD or HPAEC combined with PAD or GC with FID or MS, NMR, TLC, HP-TLC or MALDI TOF.

The supernatant concentration over whole broth concentration ratio as used herein is defined as the division of the supernatant concentration as measured and described herein to the whole broth concentration as measured and described herein, wherein the supernatant concentration forms the numerator and the whole broth concentration forms the denominator of the division. As such, such ratio can range from 0.1 to 3. In the methods of the present invention the ratio of supernatant concentration over whole broth concentration can be 0.1; 0.2; 0.3; 0.4; 0.5; 0.6; 0.7; 0.8; 0.9; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9 or 3. When the cells have a lower concentration of product compared to the supernatant concentration, such ratio ranges from higher than 0.5 to 3, more specifically such ratio can be 0.5; 0.6; 0.7; 0.8; 0.9; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9 or 3.

The term “precursor” as used herein refers to substances which are taken up or synthetized by the cell for the specific production of a sialylated oligosaccharide. In this sense a precursor can be an acceptor as defined herein, but can also be another substance, metabolite, which is first modified within the cell as part of the biochemical synthesis route of the sialylated oligosaccharide. Examples of such precursors comprise the acceptors as defined herein, and/or glucose, galactose, fructose, glycerol, sialic acid, fucose, mannose, maltose, sucrose, lactose, glucose-1-phosphate, galactose-1-phosphate, UDP-glucose, UDP-galactose, glucose-6-phosphate, fructose-6-phosphate, fructose-1,6-bisphosphate, glycerol-3-phosphate, dihydroxyacetone, glyceraldehyde-3-phosphate, dihydroxyacetone-phosphate, glucosamine-6-phosphate, glucosamine, N-acetylglucosamine-6-phosphate, N-acetylglucosamine, N-acetylmannosamine, N-acetylmannosamine-6-phosphate, UDP-N-acetylglucosamine, N-acetylglucosamine-1-phosphate, N-acetylneuraminic acid (sialic acid), N-acetyl-Neuraminic acid-9 phosphate, CMP-sialic acid, mannose-6-phosphate, mannose-1-phosphate, GDP-mannose, GDP-4-dehydro-6-deoxy-α-D-mannose, and/or GDP-fucose.

The term “acceptor” as used herein refers to oligosaccharides which can be modified by a sialyltransferase. Examples of such acceptors are lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.

### DETAILED DESCRIPTION OF THE INVENTION

In a first embodiment, the present invention provides a method for the production of sialylated oligosaccharide by a genetically modified cell. The method comprises the following steps.

A cell capable of producing said sialylated oligosaccharide is provided, wherein the cell comprises at least one nucleic acid sequence coding for an enzyme involved in sialylated oligosaccharide synthesis. The cell is genetically modified for i) overexpression of an endogenous membrane protein, ii) expression or overexpression of a homologous membrane protein, and/or iii) expression or overexpression of a heterologous membrane protein. This cell is cultured in a medium under conditions permissive for the production of the desired sialylated oligosaccharide. Optionally the sialylated oligosaccharide is separated from the cultivation as explained herein.

In a preferred embodiment, the cell is genetically modified for the production of sialylated oligosaccharide and said genetically modified cell excretes sialylated oligosaccharide at a ratio of the supernatant concentration to whole broth concentration higher than 0.5.

In the methods of the present invention the ratio of supernatant concentration over whole broth concentration can be 0.1; 0.2; 0.3; 0.4; 0.5; 0.6; 0.7; 0.8; 0.9; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9 or 3. When the cells have a lower concentration of product compared to the supernatant concentration, such ratio ranges from higher than 0.5 to 3, more specifically such ratio can be 0.5; 0.6; 0.7; 0.8; 0.9; 1.0; 1.1; 1.2; 1.3; 1.4; 1.5; 1.6; 1.7; 1.8; 1.9; 2.0; 2.1; 2.2; 2.3; 2.4; 2.5; 2.6; 2.7; 2.8; 2.9 or 3.

In an additional or alternative preferred embodiment, the cell is genetically modified for the production of sialylated oligosaccharide and said genetically modified cell has an enhanced production of sialylated oligosaccharide compared to a cell with the same genetic makeup but lacking the i) overexpression of the endogenous membrane protein, ii) expression or overexpression of the homologous membrane protein and/or iii) expression or overexpression of the heterologous membrane protein, respectively.

In the method of the invention described herein the membrane protein is either an endogenous protein with a modified expression, preferably said endogenous protein is overexpressed; or the membrane protein is a homologous or a heterologous protein, which can be expressed by the cell. The heterologous or homologous membrane protein will then be introduced and expressed, preferably overexpressed. In another embodiment, the endogenous protein can have a modified expression in the cell which also expresses a heterologous membrane protein. In another embodiment, modified expression of an endogenous membrane protein comprises modified expression of other proteins that map in the same operon of said endogenous membrane protein and/or share common control sequences for expression. In another embodiment, the membrane protein is expressed together with conterminal proteins that share the same regulon. In another embodiment, when the membrane protein is an inner membrane transporter (complex), the membrane protein is expressed together with one or more outer membrane transporter(s). In an alternative embodiment, when the membrane protein is an outer membrane transporter, the membrane protein is expressed together with one or more inner membrane protein(s). In an alternative embodiment, the membrane protein is expressed with one or more inner membrane proteins and/or one or more outer membrane proteins.

In another preferred embodiment according to the present invention, the membrane protein used in the present invention comprises
i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
iv) an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid; or
v) an amino acid sequence encoding a membrane transporter chosen from the list of SEQ ID NOs 1 to 21, 37 to 93 or 99 to 122 or a homolog having at least 80% sequence identity to the full length of any one of SEQ NOs 1 to 21, 37 to 93 or 99 to 122 and providing improved production and/or efflux of sialylated oligosaccharides.

The host cell used herein is preferably genetically modified for the production of sialylated oligosaccharide. In a further preferred embodiment, the cell used herein comprises a recombinant sialyltransferase capable of modifying lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.

The genetically modified cell capable of producing sialylated oligosaccharide is a cell comprising at least one nucleic acid sequence coding for an enzyme for sialylated oligosaccharide synthesis. Preferably, said cell comprises a biosynthetic pathway to produce a sialic acid monosaccharide nucleotide donor (typically CMP-sialic acid, also known as CMP-N-acetylneuraminic acid) suitable to be transferred by the corresponding sialyltransferase. The genetically modified cell can produce CMP-sialic acid in two ways. In one way, exogenously added sialic acid is internalized actively or passively, preferably actively by a sialic acid permease, more preferably by that encoded by nanT, and subsequently converted to CMP-sialic acid by a CMP-sialic acid synthetase, e.g. encoded by a heterologous neuA. In another way the internally available UDP-GlcNAc is utilized, by expressing heterologous neuC, neuB and neuA that convert it to CMP-sialic acid via ManNAc and sialic acid as intermediates.

In another way, the host cell used herein is optionally genetically modified for the production of sialylated oligosaccharide, wherein said host cell is modified to express the genes that catalyze de novo synthesis of CMP-N-acetylneuraminic acid. Said de novo synthesis of CMP-N-acetylneuraminic acid is started from fructose-6P and catalyzed by a mutated fructose-6-P-aminotransferase (glmS) into glucosamine-6P, a glucosamine-6-P-aminotransferase (e.g. a heterologous GNA1) into N-acetylglucosamine-6P, a native phosphatase into GlcNAc, an N-acetylglucosamine-2-epimerase (e.g. a heterologous AGE) into ManNAc, a sialic acid synthase (e.g. a heterologous neuB) into sialic acid and an CMP-sialic acid synthetase (e.g. a heterologous neuA) into CMP-sialic acid. Alternatively, de novo synthesis of CMP-N-acetylneuraminic acid is started from the internally available UDP-GlcNAc and catalyzed by an UDP-N-acetylglucosamine epimerase (e.g. a heterologous neuC) into ManNAc, a sialic acid synthase (e.g. a heterologous neuB) into sialic acid and finally an CMP-sialic acid synthetase (e.g. a heterologous neuA) into CMP-sialic acid. Alternatively, internally available UDP-GlcNAc is catalyzed by an UDP-N-acetylglucosamine-2-epimerase into ManNAc, further catalyzed by a ManNAc kinase into ManNAc-6P, further catalyzed by a sialic acid synthetase into Neu5Ac-9P, further catalyzed by a sialic acid phosphatase into sialic acid which is finally converted into CMP-sialic acid by a CMP-sialic acid synthetase. Preferably, said host cell is further modified to express one or more genes encoding for the enzymes of the de novo synthesis of CMP-N-acetylneuraminic acid (also known as CMP-sialic acid). In the meantime, the cell's catabolic activity on sialic acid and its precursor is suppressed by reducing/inactivating/deletion of the sialic acid aldolase gene (nanA) and/or the ManNAc kinase gene (nanK). The internalized carbohydrate precursor can be the subject of glycosylation other than sialylation, e.g. N-acetylglucosaminylation, galactosylation and/or fucosylation before being sialylated as described above.

As known in the art, the fermentative production comprising a genetically modified cell can occur in the following way. An exogenously added precursor can be internalized from the culture medium into the cell where it is converted to the sialylated oligosaccharide of interest in a reaction comprising enzymatic sialylation mediated by an appropriate sialyltransferase. On one mode of action, the internalization can take place via a passive transport mechanism during which the exogenous precursor diffuses passively across the plasma membrane of the cell. The flow is then directed by the concentration difference in the extra- and intracellular space with respect to the precursor molecule to be internalized, which precursor is passing from the place of higher concentration to the zone of lower concentration tending towards equilibrium. In another mode of action, the internalization of the exogenous precursor in the cell is accomplished by an active transport mechanism, during which the exogenous precursor diffuses across the plasma membrane of the cell under the influence of a transporter protein or permease of the cell. As known in the art, lactose permease (LacY) has specificity towards mono- or disaccharide selected from galactose, N-acetyl-glucosamine, lactose or another galactosylated monosaccharide, an N-acetylglucosaminylated monosaccharide and glycosidic derivatives thereof. All these carbohydrate derivatives can be easily taken up by a cell having a LacY permease by means of an active transport and, when the cell lacks the enzymes that could degrade the acceptor, accumulate in the cell before being glycosylated as e.g. described in WO 01/04341, WO 2013/182206 and WO 2014/048439. It is further also known that the specificity towards the sugar moiety of the substrate to be internalized can be altered by mutation by means of known recombinant DNA techniques. In a preferred embodiment, the exogenously added precursor is lactose, and its internalization takes place via an active transport mechanism mediated by a lactose permease of the cell, more preferably LacY. Being internalized in the cell, the acceptor is sialylated by means of a sialyltransferase expressed by a heterologous gene or nucleic acid sequence which is introduced into the cell by known techniques, e.g. by integrating it into the chromosome of the cell or using an expression vector.

A ‘sialylation pathway’ is a biochemical pathway consisting of the enzymes and their respective genes, L-glutamine-D-fructose-6-phosphate aminotransferase, glucosamine-6-phosphate deaminase, phosphoglucosamine mutase, N-acetylglucosamine-6-phosphate deacetylase, N-acetylglucosamine epimerase, UDP-N-acetylglucosamine 2-epimerase, N-acetylglucosamine-6P 2-epimerase, glucosamine 6-phosphate N-acetyltransferase, N-Acetylglucosamine-6-phosphate phosphatase, N-acetylmannosamine-6-phosphate phosphatase, N-acetylmannosamine kinase, phosphoacetylglucosamine mutase, N-acetylglucosamine-1-phosphate uridyltransferase, glucosamine-1-phosphate acetyltransferase, sialic acid synthase, N-acetylneuraminate lyase, N-acylneuraminate-9-phosphate synthase, N-acylneuraminate phosphate phosphatase, and/or CMP-sialic acid synthase, combined with a sialyltransferase leading to α 2,3; α 2,6, or α 2,8 sialylated oligosaccharides or sialylated oligosaccharide containing bioproduct. The host cell used herein is optionally genetically modified to import lactose in the cell, by the introduction and/or overexpression of a lactose permease. Said lactose permease is for example encoded by the lacY gene or the lac12 gene. Alternatively, the host cell used herein is optionally genetically modified for the de novo production of lactose within said cell.

In a more preferred embodiment of the present invention, when said membrane protein is a siderophore exporter, said siderophore exporter is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides.

In another preferred embodiment of the present invention said membrane protein is selected from SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 and providing improved production and/or efflux of sialylated oligosaccharides. As used herein, a protein having an amino acid sequence having at least 80% sequence identity to any of the enlisted membrane proteins is to be understood as that the sequence has 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5%, 99.6%, 99.7%, 99.8%, 99.9% sequence identity to the full length of the amino acid sequence of the respective membrane protein.

The amino acid sequence of such membrane protein can be a sequence chosen from SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 of the attached sequence listing, or an amino acid sequence that has at least 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% sequence identity to the full length amino acid sequence of any one of SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 and providing improved production and/or efflux of sialylated oligosaccharides.

In a more preferred alternative embodiment of the present invention, when said membrane protein is an ABC transporter, said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides. The amino acid sequence of such membrane protein can be a sequence chosen from SEQ ID NOs 18, 15, 19 or 63 of the attached sequence listing, or an amino acid sequence that has at least 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% sequence identity to the full length amino acid sequence of any one of SEQ ID NOs SEQ ID NOs 18, 15, 19 or 63 and providing improved production and/or efflux of sialylated oligosaccharides.

In a more preferred alternative embodiment of the present invention, when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides. The amino acid sequence of such membrane protein can be a sequence chosen from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 of the attached sequence listing, or an amino acid sequence that has at least 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% sequence identity to the full length amino acid sequence of any one of SEQ ID NOs SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides.

In a more preferred alternative embodiment of the present invention, when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides. The amino acid sequence of such membrane protein can be a sequence chosen from SEQ ID NOs 2, 1, 3, 16, 17 or 62 of the attached sequence listing, or an amino acid sequence that has at least 80% sequence identity, 80%, 81%, 82%, 83%, 84%, 85%, 86%, 87%, 88%, 89%, 90%, 91%, 92%, 93%, 94%, 95%, 95.5%, 96%, 96.5%, 97%, 97.5%, 98%, 98.5%, 99%, 99.5% sequence identity to the full length amino acid sequence of any one of SEQ ID NOs SEQ ID NOs 2, 1, 3, 16, 17 or 62 and providing improved production and/or efflux of sialylated oligosaccharides.

In a more preferred alternative embodiment of the present invention, when said membrane protein is entS from E. coli K12 MG1655 with SEQ ID NO 9, said entS is expressed together with any one or more of the ferric enterobactin ABC transporter encoding proteins comprising fepB, fepC, fepG and fepD.

In a more preferred alternative or additional embodiment of the present invention, when said membrane protein is entS from E. coli K12 MG1655 with SEQ ID NO 9, said entS is expressed together with outer membrane proteins including TolC. In a more preferred alternative embodiment, when said membrane protein is the outer membrane protein TolC, said TolC is expressed together with any one or more of entS, AcrAB, emrYK and/or emrAB.

In a more preferred alternative embodiment, when said membrane is oppF from E. coli K12 MG1655 with SEQ ID NO 18, said oppF is expressed together with any one or more other subunits of the murein tripeptide ABC transporter comprising oppB with SEQ ID NO 87, oppC with SEQ ID NO 88 and oppD with SEQ ID NO 89 and/or with oppA. In a more preferred alternative embodiment, when said membrane is oppB from E. coli K12 MG1655 with SEQ ID NO 87, said oppB is expressed together with any one or more other subunits of the murein tripeptide ABC transporter comprising oppF with SEQ ID NO 18, oppC with SEQ ID NO 88 and oppD with SEQ ID NO 89 and/or with oppA. In a more preferred alternative embodiment, when said membrane is oppC from E. coli K12 MG1655 with SEQ ID NO 88, said oppC is expressed together with any one or more other subunits of the murein tripeptide ABC transporter comprising oppB with SEQ ID NO 87, oppF with SEQ ID NO 18 and oppD with SEQ ID NO 89 and/or with oppA. In a more preferred alternative embodiment, when said membrane is oppD from E. coli K12 MG1655 with SEQ ID NO 89, said oppD is expressed together with any one or more other subunits of the murein tripeptide ABC transporter comprising oppB with SEQ ID NO 87, oppC with SEQ ID NO 88 and oppF with SEQ ID NO 18 and/or with oppA.

In a more preferred alternative embodiment, when said membrane protein is ImrA from Lactococcus lactis subsp. lactis bv. Diacetylactis with SEQ ID NO 15, said ImrA is expressed together with ImrB.

In a more preferred alternative embodiment, when said membrane protein is Blon_0247 from B. longum subsp. Infantis (strain ATCC 15697) with SEQ ID NO 20, said Blon_0247 is expressed together with Blon_0245 from B. longum subsp. Infantis (strain ATCC 15697) with SEQ ID NO 21.

In a more preferred alternative embodiment, when said membrane protein is Blon_0245 from B. longum subsp. Infantis (strain ATCC 15697) with SEQ ID NO 21, said Blon_0245 is expressed together with Blon_0247 from B. longum subsp. Infantis (strain ATCC 15697) with SEQ ID NO 20.

In a more preferred alternative embodiment, when said membrane protein is Blon2331 from B. longum subsp. Infantis (strain ATCC 15697) with SEQ ID NO 99, said Blon2331 is expressed together with Blon2332.

In a more preferred alternative embodiment, when said membrane protein is Bjnodj from Bradyrhizobium japonicum USDA 110 with SEQ ID NO 93, said Bjnodj is expressed together with nodulation factor nodi.

In a more preferred alternative embodiment, when said membrane protein is gsiA from E. coli K12 MG1655 with SEQ ID NO 63, said gsiA is expressed together with any one or more of iaaA, gsiB, gsiC with SEQ ID NO 85 and/or gsiD with SEQ ID NO 86. In a more preferred alternative embodiment, when said membrane protein is gsiC from E. coli K12 MG1655 with SEQ ID NO 85, said gsiC is expressed together with any one or more of iaaA, gsiA with SEQ ID NO 63, gsiB and/or gsiD with SEQ ID NO 86. In a more preferred alternative embodiment, when said membrane protein is gsiD from E. coli K12 MG1655 with SEQ ID NO 86, said gsiD is expressed together with any one or more of iaaA, gsiA with SEQ ID NO 63, gsiB and/or gsiC with SEQ ID NO 85.

In a more preferred alternative embodiment, when said membrane protein is wzx with any one of SEQ ID NOs 72, 73, 74 or 75, said wzx is expressed together with any one or more of wza, wzb and/or wzc.

In a more preferred alternative embodiment, when said membrane protein is mdlA from E. coli K12 MG1655 with SEQ ID NO 83, said mdlA is expressed together with mdlB from E. coli K12 MG1655 with SEQ ID NO 84.

In a more preferred alternative embodiment, when said membrane protein is mdlB from E. coli K12 MG1655 with SEQ ID NO 84, said mdlB is expressed together with mdlA from E. coli K12 MG1655 with SEQ ID NO 83.

In a more preferred alternative embodiment, when said membrane protein is uidB from E. coli K12 W3110 with SEQ ID NO 112, said uidB is expressed together with any one or more of uidA and uidC.

In a more preferred alternative embodiment, when said membrane protein is melB from E. coli K12 W3110 with SEQ ID NO 109, said melB is expressed together with melA.

In a more preferred alternative embodiment, when said membrane protein is AcrB from E. coli K12 W3110 with SEQ ID NO 102, said AcrB is expressed together with any one or more of AcrA and TolC.

In a further preferred aspect, the method for the production of sialylated oligosaccharide as described herein further comprises at least one of the following steps:
i) Adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
ii) Adding a precursor feed in a continuous manner to the culture medium over the course of 1 day, 2 days, 3 days, 4 days, 5 days by means of a feeding solution;
iii) Adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
iv) Said method resulting in a sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

Preferably the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.

Preferably the precursor is a precursor as defined herein, and for example being chosen from the list comprising lactose, lacto-N-biose, N-acetyllactosamine.

In an exemplary embodiment the present invention provides for a method for the production of sialyllactose as described herein and further comprises at least one of the following steps:
i) Adding to the culture medium a lactose feed comprising at least 50, more preferably at least 75, more preferably at least 100, more preferably at least 120, more preferably at least 150 gram of lactose per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said lactose feed;
ii) Adding a lactose feed in a continuous manner to the culture medium over the course of 1 day, 2 days, 3 days, 4 days, 5 days by means of a feeding solution;
iii) Adding a lactose feed in a continuous manner to the culture medium over the course of 1 day, 2 days, 3 days, 4 days, 5 days by means of a feeding solution and wherein the concentration of said lactose feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
iv) Said method resulting in a sialyllactose concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

In another aspect the precursor feed is accomplished by adding precursor to the cultivation medium in a concentration, such that throughout the production phase of the cultivation a precursor concentration of at least 5 mM, preferably 10 mM or 30 mM is obtained.

In a further embodiment of the methods described herein the host cells are cultivated for at least about 60, 80, 100, or about 120 hours or in a continuous manner.

In another embodiment of the methods described herein a carbon and energy source, preferably sucrose, glucose, fructose, glycerol, maltose, maltodextrine, trehalose, polyols, starch, succinate, malate, pyruvate, lactate, ethanol, citrate, and/or lactose, is also added, preferably continuously to the culture medium, preferably with the precursor.

In a preferred embodiment, a carbon-based substrate is provided, preferably sucrose, in the culture medium for 3 or more days, preferably up to 7 days; and/or provided, in the culture medium, at least 100, advantageously at least 105, more advantageously at least 110, even more advantageously at least 120 grams of sucrose per liter of initial culture volume in a continuous manner, so that the final volume of the culture medium is not more than three-fold, advantageously not more than two-fold, more advantageously less than two-fold of the volume of the culturing medium before the culturing.

Preferably, when performing the method as described herein, a first phase of exponential cell growth is provided by adding a carbon-based substrate, preferably glucose or sucrose, to the culture medium before the precursor is added to the culture medium in a second phase.

In an alternative preferable embodiment, in the method as described herein, the precursor is added already in the first phase of exponential growth together with the carbon-based substrate.

In another embodiment, the method as described herein produces only one sialylated oligosaccharide as described herein. Preferably the sialylated oligosaccharide is one of the group consisting of 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Acα-2,3Gal beta-1,3GalNAc beta-1,3Gala-1,4Gal beta-1,4Gal), sialylated tetrasaccharide (Neu5Acα-2,3Gal beta-1,4GlcNAc beta-14GlcNAc), pentasaccharide LSTD (Neu5Acα-2,3Gal beta-1,4GlcNAc beta-1,3Gal beta-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyl lactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s).

In an alternative embodiment the method as described herein is producing a mixture of sialyllactoses.

In a further alternative embodiment, the method as described herein is producing a mixture of sialylated oligosaccharides as described herein, preferably selected from the group of 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Acα-2,3Gal beta-1,3GalNAc beta-1,3Gala-1,4Gal beta-1,4Gal), sialylated tetrasaccharide (Neu5Acα-2,3Gal beta-1,4GlcNAc beta-14GlcNAc), pentasaccharide LSTD (Neu5Acα-2,3Gal beta-1,4GlcNAc beta-1,3Gal beta-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s).

Such mixture can comprise at least two oligosaccharides preferably chosen from the group consisting of 3-SL (3′-sialyllactose), 3′-sialyllactosamine, 6-SL (6′-sialyllactose), 6′-sialyllactosamine, oligosaccharides comprising 6′-sialyllactose, SGG hexasaccharide (Neu5Acα-2,3Gal beta-1,3GalNAc beta-1,3Gala-1,4Gal beta-1,4Gal), sialylated tetrasaccharide (Neu5Acα-2,3Gal beta-1,4GlcNAc beta-14GlcNAc), pentasaccharide LSTD (Neu5Acα-2,3Gal beta-1,4GlcNAc beta-1,3Gal beta-1,4Glc), sialylated lacto-N-triose, sialylated lacto-N-tetraose, sialyllacto-N-neotetraose, monosialyllacto-N-hexaose, disialyllacto-N-hexaose I, monosialyllacto-N-neohexaose I, monosialyllacto-N-neohexaose II, disialyllacto-N-neohexaose, disialyllacto-N-tetraose, disialyllacto-N-hexaose II, sialyllacto-N-tetraose a, disialyllacto-N-hexaose I, sialyllacto-N-tetraose b, 3′-sialyl-3-fucosyllactose, disialomonofucosyllacto-N-neohexaose, monofucosylmonosialyllacto-N-octaose (sialyl Lea), sialyllacto-N-fucohexaose II, disialyllacto-N-fucopentaose II, monofucosyldisialyllacto-N-tetraose and oligosaccharides bearing one or several sialic acid residu(s).

In the method described herein the genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal, all as described herein.

In a specific exemplary embodiment, the method of the invention provides the production of sialylated oligosaccharide, preferably in high yield. The method comprises the step of culturing or fermenting, an in aqueous culture or fermentation medium containing precursor, a genetically modified cell, preferably an E. coli, more preferably an E. coli cell modified by knocking out the genes lacZ, lacY lacA, nagA, nagB, nanA, nanE, nanK, glgC, agp, pfkA, pfkB, pgi, arcA, iclR, wcaJ, Ion and/or thyA. Even more preferably, additionally the E. coli lacY gene, a sucrose permease cscB from E. coli W, a fructose kinase gene (frk) originating from Zymomonas mobilis and a sucrose phosphorylase (SP) originating from Bifidobacterium adolescentis can knocked in into the genome and expressed constitutively. The constitutive promoters originate from the promoter library described by De Mey et al. (BMC Biotechnology, 2007). These genetic modifications are also described in WO2016075243, WO2012007481, WO2013087884, and WO2018122225. Additionally, the modified E. coli cell has a recombinant gene which encodes a single sialyltransferase, in an exemplary embodiment this can be an 2,3-sialyltransferase, that is capable of modifying lactose to produce 3-sialyllactose (3′-SL). The cell furthermore comprises a recombinant gene which encodes the expression of any one of the membrane proteins as described herein.

In a preferred embodiment of the production of sialylated oligosaccharide/sialylated lactoses by a genetically modified microorganism, the microorganism able to produce sialylated oligosaccharide is an E. coli, preferably of LacY+LacZ-genotype carrying neuBCA. The heterologous sialyltransferase gene in the microorganism is preferably an α-2,3- or an α-2,6-sialyl transferase with the aid of which, from the exogenously added lactose as carbohydrate acceptor, 3′-SL or 6′-SL is produced, respectively. Such a microorganism is disclosed e.g. in WO 2007/101862, Fierfort et al, J. Biotechnol, 134, 261 (2008), Drouillard et al. Carbohydr. Res. 345, 1394 (2010) and WO 2017/101958.

Another aspect of the present invention provides a host cell genetically modified for the production of sialylated oligosaccharide, wherein the host cell comprises at least one nucleic acid sequence coding for an enzyme for sialylated oligosaccharide synthesis, and wherein said cell is genetically modified for i) overexpression of an endogenous membrane protein, ii) expression or overexpression of a homologous membrane protein, and/or iii) expression or overexpression of a heterologous membrane protein, wherein said membrane protein comprises:
i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TN1, 05C0R, 07Y9F, 05CSH, 05QRD, 05EDF, 05C6X, 08NGX, 05C2C, 07FU4, 07U9Z, 080SS, 07SF1, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
iv) an amino acid sequence encoding a Sugar Efflux Transporter, preferably said membrane protein is an MFS transporter comprising the conserved domain L[FY]AxNR[HN]Y (SEQ ID NO 98), wherein x can by any distinct amino acid; or
v) an amino acid sequence encoding a membrane transporter chosen from the list of SEQ ID NOs 1 to 21, 37 to 93 or 99 to 122 or a homolog having at least 80% sequence identity to the full length of any one of SEQ NOs 1 to 21, 37 to 93 or 99 to 122 and providing improved production and/or efflux of sialylated oligosaccharides.

Alternatively or preferably, when the membrane protein is a siderophore exporter, said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides.

Alternatively or preferably, when the membrane protein is an ABC transporter, said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides.

Alternatively or preferably, when the membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides.

Alternatively or preferably, when the membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.

In another preferred embodiment of the present invention said membrane protein is selected from SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 and providing improved production and/or efflux of sialylated oligosaccharides. Another aspect provides for a cell to be stably cultured in a medium, which cell is adjusted for the production of sialylated oligosaccharide. The cell is transformed to comprise at least one nucleic acid sequence coding for an enzyme for sialylated oligosaccharide synthesis and the cell in addition comprises i) an overexpression of an endogenous membrane protein and/or ii) an expression or an overexpression of a homologous or heterologous membrane protein. The membrane protein is chosen from any of the membrane proteins as described herein.

Optionally, the cell is transformed to comprise at least one nucleic acid sequence encoding a protein selected from the group consisting of lactose transporter, N-acetylneuraminic acid transporter, fucose transporter, transporter for a nucleotide-activated sugar wherein said transporter internalizes a to the medium added precursor for sialylated oligosaccharide synthesis.

In the methods described herein the cell can be a cell of any organism. The term ‘organism’ or ‘cell’ as used herein refers to a microorganism chosen from the list consisting of a bacterium, a yeast or a fungus, or, refers to a plant cell, animal cell, a mammalian cell, an insect cell and a protozoal cell. The latter bacterium preferably belongs to the phylum of the Proteobacteria or the phylum of the Firmicutes or the phylum of the Cyanobactria or the phylum Deinococcus-Thermus. The latter bacterium belonging to the phylum Proteobacteria belongs preferably to the family Enterobacteriaceae, preferably to the species Escherichia coli. The latter bacterium preferably relates to any strain belonging to the species Escherichia coli such as but not limited to Escherichia coli B, Escherichia coli C, Escherichia coli W, Escherichia coli K12, Escherichia coli Nissle. More specifically, the latter term relates to cultivated Escherichia coli strains—designated as E. coli K12 strains—which are well-adapted to the laboratory environment, and, unlike wild type strains, have lost their ability to thrive in the intestine. Well-known examples of the E. coli K12 strains are K12 Wild type, W3110, MG1655, M182, MC1000, MC1060, MC1061, MC4100, JM101, NZN111 and AA200. Hence, preferably the present invention specifically relates to a mutated and/or transformed Escherichia coli strain as indicated above wherein said E. coli strain is a K12 strain. More specifically, the present invention relates to a mutated and/or transformed Escherichia coli strain as indicated above wherein said K12 strain is E. coli MG1655. The latter bacterium belonging to the phylum Firmicutes belongs preferably to the Bacilli, preferably from the species Bacillus. The latter yeast preferably belongs to the phylum of the Ascomycota or the phylum of the Basidiomycota or the phylum of the Deuteromycota or the phylum of the Zygomycetes. The latter yeast belongs preferably to the genus Saccharomyces, Pichia, Komagataella, Hansunella, Kluyveromyces, Yarrowia, Stormerella, Eremothecium, Zygosaccharomyces or Debaromyces. The latter fungus belongs preferably to the genus Rhizopus, Dictyostelium, Penicillium, Mucor or Aspergillus. “Plant cells” includes cells of flowering and non-flowering plants, as well as algal cells, for example Chlamydomonas, Chlorella, etc. Preferably, said plant cell is a tobacco, alfalfa, rice, tomato, corn, maize or soybean cell; said mammalian cell is a CHO cell or a HEK cell; said insect cell is an S. frugiperda cell and said protozoal cell is a L. tarentolae cell.

In a preferred embodiment the cell is a cell of a microorganism, wherein more preferably said microorganism is a bacterium or a yeast. In a more preferred embodiment, the microorganism is a bacterium, most preferably Escherichia coli. Examples using such E. coli are described herein.

In another more preferred embodiment, the microorganism is yeast. Examples using yeast for the production of sialylated oligosaccharides and useable in the present invention are e.g. described in WO18122225.

It is generally preferred that the cell's catabolic pathway for selected mono-, di- or oligosaccharides is at least partially inactivated, the mono-, di-, or oligosaccharides being involved in and/or required for the synthesis of sialylated oligosaccharides.

In a further embodiment the present invention provides a method for the production of sialylated oligosaccharide, wherein a cell as described herein is used for culturing in a medium under conditions permissive for the production of said sialylated oligosaccharide. The sialylated oligosaccharide is then separated from the cultivation.

As used herein, conditions permissive for the production are to be understood to be conditions relating to physical or chemical parameters enabling growth of and living cells, including but not limited to temperature, pH, pressure, osmotic pressure and product/educt concentration. Preferably, such permissive conditions may include temperature range of 30+/−20° C., a pH range of 7+/−3.

The cell according to the invention produces sialylated oligosaccharide as described herein. The sialylated oligosaccharide is preferably chosen from the group consisting of 6′-sialyllactose, 3′-sialyllactose, 3-fucosyl-3′-sialyllactose (3′-O-sialyl-3-O-fucosyllactose, FSL), 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-LNH (SLNH), sialyl-lacto-N-hexaose (SLNH), sialyl-lacto-N-neohexaose I (SLNH-1), sialyl-lacto-N-neohexaose 11 (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose. The cell can also produce a mixture of sialylated oligosaccharides. Such mixture comprises at least two sialylated oligosaccharides preferably chosen from the group consisting of 6′-sialyllactose, 3′-sialyllactose, 3-fucosyl-3′-sialyllactose (3′-O-sialyl-3 fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-1), sialyl-lacto-N-neohexaose 11 (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.

Another aspect of the present invention provides for the use of a membrane protein selected from the group of membrane proteins as defined herein in the fermentative production of sialylated oligosaccharide. The sialylated oligosaccharide is an oligosaccharide as described herein. The cell can also produce a mixture of sialylated oligosaccharides. Such mixture comprises at least two sialylated oligosaccharides as described herein.

In a further aspect, the present invention provides for the use of a cell as defined herein, in a method for the production of sialylated oligosaccharide as described herein.

In yet another aspect, the present invention provides for the use of a cell as defined herein for the production of sialylated oligosaccharide as defined herein.

Furthermore, the invention also relates to the sialylated oligosaccharide obtained by the methods according to the invention, as well as to the use of a polynucleotide, the vector, host cells, microorganisms or the polypeptide as described herein for the production of sialylated oligosaccharide or a mix of sialylated oligosaccharides. The sialylated oligosaccharide or the mix may be used as food additive, prebiotic, symbiotic, for the supplementation of baby food, adult food or feed, or as either therapeutically or pharmaceutically active compound. With the novel methods, sialylated oligosaccharide can easily and effectively be provided, without the need for complicated, time and cost consuming synthetic processes.

In a preferred embodiment of the methods of the invention the produced sialylated oligosaccharide or mix of sialylated oligosaccharides is separated from the culture.

As used herein, the term “separating” means harvesting, collecting or retrieving the sialylated oligosaccharide from the host cell and/or the medium of its growth as explained herein.

Sialylated oligosaccharide can be separated in a conventional manner from the culture or aqueous culture medium, in which the mixture was made. In case the sialylated oligosaccharide is still present in the cells producing the sialylated oligosaccharide, conventional manners to free or to extract the sialylated oligosaccharide out of the cells can be used, such as cell destruction using high pH, heat shock, sonication, French press, homogenisation, enzymatic hydrolysis, chemical hydrolysis, solvent hydrolysis, detergent, hydrolysis, . . . . The culture medium, reaction mixture and/or cell extract, together and separately called sialylated oligosaccharide containing mixture or culture, can then be further used for separating the sialylated oligosaccharide.

Typically oligosaccharides, and sialylated oligosaccharide being an oligosaccharide, are purified by first removing macro components, i.e. first the cells and cell debris, then the smaller components, i.e. proteins, endotoxins and other components between 1000 Da and 1000 kDa and then the oligosaccharide is desalted by means of retaining the oligosaccharide with a nanofiltration membrane or with electrodialysis in a first step and ion exchange in a second step, which consists of a cation exchange resin and anion exchange resin, wherein most preferably the cation exchange chromatography is performed before the anion exchange chromatography. These steps do not separate sugars with a small difference in degree of polymerization from each other. Said separation is done for instance by chromatographical separation.

Separation preferably involves clarifying the sialylated oligosaccharide containing mixtures to remove suspended particulates and contaminants, particularly cells, cell components, insoluble metabolites and debris produced by culturing the genetically modified cell and/or performing the enzymatic reaction. In this step, the sialylated oligosaccharide containing mixture can be clarified in a conventional manner. Preferably, the sialylated oligosaccharide containing mixture is clarified by centrifugation, flocculation, decantation and/or filtration. A second step of separating the sialylated oligosaccharide from the sialylated oligosaccharide containing mixture preferably involves removing substantially all the proteins, as well as peptides, amino acids, RNA and DNA and any endotoxins and glycolipids that could interfere with the subsequent separation step, from the sialylated oligosaccharide containing mixture, preferably after it has been clarified. In this step, proteins and related impurities can be removed from the sialylated oligosaccharide containing mixture in a conventional manner. Preferably, proteins, salts, byproducts, colour and other related impurities are removed from the sialylated oligosaccharide containing mixture by ultrafiltration, nanofiltration, reverse osmosis, microfiltration, activated charcoal or carbon treatment, tangential flow high-performance filtration, tangential flow ultrafiltration, affinity chromatography, ion exchange chromatography (such as but not limited to cation exchange, anion exchange, mixed bed ion exchange), hydrophobic interaction chromatography and/or gel filtration (i.e., size exclusion chromatography), particularly by chromatography, more particularly by ion exchange chromatography or hydrophobic interaction chromatography or ligand exchange chromatography. With the exception of size exclusion chromatography, proteins and related impurities are retained by a chromatography medium or a selected membrane, while sialylated oligosaccharide remains in the sialylated oligosaccharide containing mixture.

Contaminating compounds with a molecular weight above 1000 Da (dalton) are removed by means of ultrafiltration membranes with a cut-off above 1000 Da to approximately 1000 kDa. The membrane retains the contaminant and the oligosaccharide goes to the filtrate. Typical ultrafiltration principles are well known in the art and are based on Tubular modules, Hollow fiber, spiral-wound or plates. These are used in cross flow conditions or as a dead-end filtration. The membrane composition is well known and available from several vendors, and are composed of PES (Polyethylene sulfone), polyvinylpyrrolidone, PAN (Polyacrylonitrile), PA (Poly-amide), Polyvinylidene difluoride (PVDF), NC (Nitrocellulose), ceramic materials or combinations thereof.

Components smaller than the oligosaccharide, for instance monosaccharides, salts, disaccharides, acids, bases, medium constituents are separated by means of a nano-filtration or/and electrodialysis. Such membranes have molecular weight cut-offs between 100 Da and 1000 Da. For an oligosaccharide such as 3′-sialyllactose or 6′-sialyllactose the optimal cut-off is between 300 Da and 500 Da, minimizing losses in the filtrate. Typical membrane compositions are well known and are for example polyamide (PA), TFC, PA-TFC, Polypiperazine-amide, PES, Cellulose Acetate or combinations thereof.

Sialylated oligosaccharide is further isolated from the culture medium and/or cell with or without further purification steps by evaporation, lyophilization, crystallization, precipitation, and/or drying, spray drying. Said further purification steps allow the formulation of sialylated oligosaccharide in combination with other oligosaccharides and/or products, for instance but not limited to the co-formulation by means of spray drying, drying or lyophilization or concentration by means of evaporation in liquid form.

In an even further aspect, the present invention also provides for a further purification of the sialylated oligosaccharide. A further purification of said sialylated oligosaccharide may be accomplished, for example, by use of (activated) charcoal or carbon, nanofiltration, ultrafiltration or ion exchange to remove any remaining DNA, protein, LPS, endotoxins, or other impurity. Alcohols, such as ethanol, and aqueous alcohol mixtures can also be used. Another purification step is accomplished by crystallization or precipitation of the product. Another purification step is to spray dry or lyophilize sialylated oligosaccharide.

The separated and preferably also purified sialylated oligosaccharide can be used as a supplement in infant formulas and for treating various diseases in newborn infants.

In a specific embodiment of the present invention a bacterial cell is provided, said cell to be stably cultured in a medium for the production of oligosaccharides, more specifically sialyllactose. This cell is transformed to comprise at least one nucleic acid sequence coding for a sialyltransferase, and the cell in addition is transformed to comprise at least one nucleic acid sequence coding for a membrane protein wherein said membrane protein comprises
i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SF1, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
iv) an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid; or
v) an amino acid sequence encoding a membrane transporter chosen from the list of SEQ ID NOs 1 to 21, 37 to 93 or 99 to 122 or a homolog having at least 80% sequence identity to the full length of any one of SEQ NOs 1 to 21, 37 to 93 or 99 to 122 and providing improved production and/or efflux of sialylated oligosaccharides.

Preferably the bacterial cell is an Escherichia coli cell.

In the above specific embodiment, the bacterial cell is preferably comprising, when said membrane protein is a siderophore exporter, a membrane protein being selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides.

Alternatively or preferably, when said membrane protein is an ABC transporter, said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides.

Alternatively or preferably, when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides.

Alternatively or preferably, when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.

In another preferred embodiment of the present invention said membrane protein is selected from SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 1, 2, 3, 4, 5, 6, 7, 8, 9, 10, 11, 12, 13, 14, 15, 16, 17, 18, 19, 20, 21, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 and providing improved production and/or efflux of sialylated oligosaccharides.

The above bacterial cell is in another preferred embodiment further transformed to comprise at least one nucleic acid sequence coding for a protein facilitating or promoting the import of precursor required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.

In another preferred embodiment, such bacterial cell is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.

The above specific cell can furthermore be used in method for the production of sialyllactose, wherein the method provides the cell described above and then cultures the cell in a medium under conditions permissive for the production of the sialyllactose. Optionally the sialyllactose can then be separated from the culture, and preferably further purified.

In a preferred embodiment of the above specific embodiment, said sialyllactose is 3′-sialyllactose and/or 6′-sialyllactose.

In the methods of the invention the culturing is preferably performed using a continuous flow bioreactor.

In an alternative embodiment, the culturing can be done in batches.

The medium used in the methods of the present invention preferably substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.

As will be shown in the examples herein, the method and the cell of the invention provide at least one of the following surprising advantages when using the membrane proteins as defined herein:
Better sialylated oligosaccharide titers (enhanced production) (g/L),
Better production rate r (g sialylated oligosaccharide /L/h),
Better cell performance index CPI (g sialylated oligosaccharide /g X),
Better specific productivity Qp (g sialylated oligosaccharide /g X /h),
Better yield on sucrose Ys (g sialylated oligosaccharide /g sucrose),
Better sucrose uptake/conversion rate Qs (g sucrose /g X /h),
Better precursor conversion/consumption rate rs (g precursor /h),
Enhanced sialylated oligosaccharide secretion or export ratio, and/or
Enhanced growth speed of the production host,
   when compared to sialylated oligosaccharide production host with an identical genetic background but lacking the expression or overexpression of the homologous or heterologous membrane protein or overexpression of the endogenous membrane protein.

Moreover, the present invention relates to the following specific embodiments:
1. Method for the production of sialylated oligosaccharide by a genetically modified cell, comprising the steps of:
   providing a cell genetically modified for the production of sialylated oligosaccharide, said cell comprising at least one nucleic acid sequence coding for an enzyme for sialylated oligosaccharide synthesis,
   said cell genetically modified for i) modified expression of an endogenous membrane protein, ii) expression of a homologous membrane protein, and/or iii) expression of a heterologous membrane protein
   wherein said modified cell excretes sialylated oligosaccharide at a ratio of the supernatant concentration to whole broth concentration higher than 0.5,
   culturing the cell in a medium under conditions permissive for the production of sialylated oligosaccharide,
   optionally separating sialylated oligosaccharide from the culture.
2. Method for the production of sialylated oligosaccharide by a genetically modified cell, comprising the steps of:
   providing a cell genetically modified for the production of sialylated oligosaccharide, said cell comprising at least one nucleic acid sequence coding for an enzyme for sialylated oligosaccharide synthesis,
   said cell genetically modified for i) modified expression of an endogenous membrane protein, ii) expression of a homologous membrane protein, and/or iii) expression of a heterologous membrane protein
   wherein said modified cell has an enhanced production of sialylated oligosaccharide compared to a cell with the same genetic makeup but lacking the i) modified expression of the endogenous membrane protein, ii) expression of the homologous membrane protein and/or iii) expression of the heterologous membrane protein, respectively,
   culturing the cell in a medium under conditions permissive for the production of sialylated oligosaccharide,
   optionally separating sialylated oligosaccharide from the culture.
3. Method according to any one of embodiment 1 or 2, wherein said modified expression in i) or expression in ii) and/or iii) is an overexpression of said membrane protein.
4. Method according to any one of embodiments 1 to 3, wherein said membrane protein comprises
   i. a) an amino acid sequence encoding a conserved domain GxSGxGKST (SEQ ID NO 94) and b) an amino acid sequence encoding a conserved domain SGGQxQRxxxxRAxxxxPK (SEQ ID NO 95) wherein x can be any distinct amino acid; or
   ii. a) an amino acid sequence encoding a conserved domain [AGMS]x[FLMVY]x[DGKNQR]xx[EGST][PRTVY][KR]x[GILMV] (SEQ ID NO 96) and b) an amino acid sequence encoding a conserved domain [LRST]xxx[AG][AFILV] (SEQ ID NO 97), wherein x can be any distinct amino acid; or
   iii. an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid; or
   iv. an amino acid sequence encoding a siderophore exporter.
5. Method according to any one of embodiments 1 to 4, wherein said siderophore exporter is part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or
   bactNOG families 05E8G, 08HFG, 089VA, 07TN1, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP.
6. Method according to any one of embodiment 1 to 5, wherein said membrane protein is selected from SEQ ID NOs 11, 1, 2, 3, 4, 5, 6, 7, 8, 9, 10, 12, 13, 14, 15, 16, 17, 18, 19, 20, 21, 22, 23, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 11, 1, 2, 3, 4, 5, 6, 7, 8, 9, 10, 12, 13, 14, 15, 16, 17, 18, 19, 20, 21, 22, 23, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122.
7. Method for the production of sialylated oligosaccharide according to any one of the previous embodiments, the method further comprising at least one of the following steps:
   i) Adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   ii) Adding a precursor feed in a continuous manner to the culture medium over the course of 1 day, 2 days, 3 days, 4 days, 5 days by means of a feeding solution;
   iii) Adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   iv) Said method resulting in a sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
8. The method of embodiment 7, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.
9. The method of any one of the embodiments 7 or 8, wherein said precursor feed is accomplished by adding precursor to the cultivation medium in a concentration, such, that throughout the production phase of the cultivation a precursor concentration of at least 5 mM, preferably 10 mM or 30 mM is obtained.
10. The method of any of the embodiments 7, 8 or 9, wherein the host cells are cultivated for at least about 60, 80, 100, or about 120 hours or in a continuous manner.
11. The method of any one of embodiments 1 to 10, wherein a precursor feed is added to the culture medium and wherein precursor is chosen from the group comprising lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.
12. The method of any one of embodiments 1 to 11, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.
13. The method of any one of embodiments 1 to 12, wherein a first phase of exponential cell growth is provided by adding a carbon-based substrate, preferably glucose or sucrose, to the culture medium before the lactose is added to the culture medium in a second phase.
14. Method according to any one of embodiments 1 to 13, wherein said sialylated oligosaccharide is 6′-sialyllactose, 3′-sialyl lactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
15. Method according to any one of the embodiments 1 to 14, wherein the method is producing a mixture of sialylated oligosaccharides.
16. Method according to any one of embodiment 1 to 15, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal, preferably the cell is an Escherichia coli cell.
17. Host cell genetically modified for the production of a sialylated oligosaccharide, wherein the host cell comprises at least one nucleic acid sequence coding for an enzyme for sialylated oligosaccharide synthesis and wherein said cell is genetically modified for i) modified expression of an endogenous membrane protein, ii) expression of a homologous membrane protein, and/or iii) expression of a heterologous membrane protein,
   wherein said membrane protein comprises
   i) a) an amino acid sequence encoding a conserved domain GxSGxGKST (SEQ ID NO 94) and b) an amino acid sequence encoding a conserved domain SGGQxQRxxxxRAxxxxPK (SEQ ID NO 95) wherein x can be any amino acid; or
   ii) a) an amino acid sequence encoding a conserved domain [AGMS]x[FLMVY]x[DGKNQR]xx[EGST][PRTVY][KR]x[GILMV] (SEQ ID NO 96) and b) an amino acid sequence encoding a conserved domain [LRST]xxx[AG][AFILV] (SEQ ID NO 97), wherein x can be any distinct amino acid; or
   iii) an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid; or
   iv) an amino acid sequence encoding a siderophore exporter.
18. Host cell according to embodiment 17, wherein said membrane protein is part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or
   bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP.
19. Cell according to any one of the embodiments 17 or 18, wherein said membrane protein is selected from SEQ ID NOs 11, 1, 2, 3, 4, 5, 6, 7, 8, 9, 10, 12, 13, 14, 15, 16, 17, 18, 19, 20, 21, 22, 23, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 11, 1, 2, 3, 4, 5, 6, 7, 8, 9, 10, 12, 13, 14, 15, 16, 17, 18, 19, 20, 21, 22, 23, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122.
20. Cell according to any one of embodiments 17 to 19, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
21. Cell according to any one of the embodiments 17 to 20, wherein the cell comprises a catabolic pathway for selected mono-, di- or oligosaccharides which is at least partially inactivated, the mono-, di-, or oligosaccharides being involved in and/or required for the synthesis of sialylated oligosaccharide.
22. Cell according to any one of the embodiments 17 to 21, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
23. Method for the production of sialylated oligosaccharide, comprising the steps of:
   a) providing a cell according to any one of the embodiments 17 to 22,
   b) culturing the cell in a medium under conditions permissive for the production of said sialylated oligosaccharide,
   c) separating said sialylated oligosaccharide from the culture.
24. Use of a membrane protein selected from the group of membrane proteins as defined in any one of the embodiments 1 to 16 in the fermentative production of sialylated oligosaccharide.
25. Use of a cell according to any one of the embodiments 17 to 22, in a method for the production of sialylated oligosaccharide.
26. Use of a cell according to any one of embodiments 24 or 25 wherein said sialylated oligosaccharide 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′ sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
27. A bacterial cell to be stably cultured in a medium for the production of oligosaccharides, said oligosaccharides being sialyllactose, the cell being transformed to comprise at least one nucleic acid sequence coding for a sialyltransferase, characterized in that: the cell in addition is transformed to comprise at least one nucleic acid sequence coding for a membrane protein wherein said membrane protein comprises
   i) a) an amino acid sequence encoding a conserved domain GxSGxGKST (SEQ ID NO 94) and b) an amino acid sequence encoding a conserved domain SGGQxQRxxxxRAxxxxPK (SEQ ID NO 95) wherein x can be any amino acid; or
   ii) a) an amino acid sequence encoding a conserved domain [AGMS]x[FLMVY]x[DGKNQR]xx[EGST][PRTVY][KR]x[GILMV] (SEQ ID NO 96); and b) an amino acid sequence encoding a conserved domain [LRST]xxx[AG][AFILV] (SEQ ID NO 97), wherein x can be any distinct amino acid; or
   iii) an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid; or
   iv) an amino acid sequence encoding a siderophore exporter, and which protein is overexpressed.
28. The bacterial cell according to embodiment 27, characterized in that the cell is an Escherichia coli cell.
29. The bacterial cell according to any one of embodiments 27 or 28, wherein said siderophore exporter is part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or
   bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP.
30. The bacterial cell according to any one of embodiments 27 to 29, wherein said membrane protein is selected from SEQ ID NOs 11, 1, 2, 3, 4, 5, 6, 7, 8, 9, 10, 12, 13, 14, 15, 16, 17, 18, 19, 20, 21, 22, 23, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 11, 1, 2, 3, 4, 5, 6, 7, 8, 9, 10, 12, 13, 14, 15, 16, 17, 18, 19, 20, 21, 22, 23, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 108, 109, 110, 111, 112, 113, 114, 115, 116, 117, 118, 119, 120, 121 or 122.
31. The bacterial cell according to any one of embodiments 17 to 22, 27 to 30, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
32. The bacterial cell according to anyone of embodiments 17 to 22, 27 to 30, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
33. A method for the production of oligosaccharides, said oligosaccharides being sialyllactose, the method comprising the steps of:
   a) providing a cell according to anyone of embodiments 17 to 22, 27 to 32,
   b) culturing the cell in a medium under conditions permissive for the production of said oligosaccharides,
   c) optionally separating said oligosaccharides from the culture.
34. The method according to any one of embodiments 1 to 16, 23 to 26 or 33, characterized in that culturing is performed using a continuous flow bioreactor.
35. The method according to any one of embodiments 1 to 16, 23 to 26, 33 or 34, characterized in that, the medium comprises substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.
36. Method according to any one of embodiments 1 to 16, 23 to 26, 33 to 35, wherein said sialyllactose is 3′-sialyllactose and/or 6′-sialyllactose.
37. Method for the production of sialylated oligosaccharide by a genetically modified cell, comprising the steps of:
   providing a cell capable of producing sialylated oligosaccharide, said cell comprising at least one nucleic acid sequence coding for an enzyme for sialylated oligosaccharide synthesis,
   said cell genetically modified for i) modified expression of an endogenous membrane protein, ii) expression of a homologous membrane protein, and/or iii) expression of a heterologous membrane protein, and wherein said membrane protein comprises a) an amino acid sequence encoding a conserved domain GxSGxGKST (SEQ ID NO 94) and b) an amino acid sequence encoding a conserved domain SGGQxQRxxxxRAxxxxPK (SEQ ID NO 95), wherein x can be any amino acid,
   culturing the cell in a medium under conditions permissive for the production of sialylated oligosaccharide,
   optionally separating sialylated oligosaccharide from the culture.
38. Method according to embodiment 37, wherein said modified expression in i) or expression in ii) and/or iii) is an overexpression of said membrane protein.
39. Method according to any one of embodiments 37 or 38, wherein membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NO 18, SEQ ID NO 15, SEQ ID NO 19 or SEQ ID NO 63.
40. Method for the production of sialylated oligosaccharide according to any one of embodiments 37 to 39, the method further comprising at least one of the following steps:
   i) adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   adding a precursor feed in a continuous manner to the culture medium over the course of 1 day, 2 days, 3 days, 4 days, 5 days by means of a feeding solution;
   iii) adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   iv) said method resulting in a sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
41. The method of embodiment 40, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.
42. The method of any one of the embodiments 40 or 41, wherein said precursor feed is accomplished by adding precursor to the cultivation medium in a concentration, such, that throughout the production phase of the cultivation a precursor concentration of at least 5 mM, preferably 10 mM or 30 mM is obtained.
43. The method of any of the embodiments 40, 41 or 42, wherein the host cells are cultivated for at least about 60, 80, 100, or about 120 hours or in a continuous manner.
44. The method of any one of embodiments 37 to 43, wherein a precursor feed is added to the culture medium and wherein said precursor is chosen from the group comprising lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.
45. The method of any one of embodiments 37 to 44, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.
46. The method of any one of embodiments 37 to 45, wherein a first phase of exponential cell growth is provided by adding a carbon-based substrate, preferably glucose or sucrose, to the culture medium before the lactose is added to the culture medium in a second phase.
47. Method according to any one of embodiments 37 to 46, wherein said sialylated oligosaccharide is 6′-sialyllactose, 3′-sialyl lactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
48. Method according to any one of the embodiments 37 to 47, wherein the method is producing a mixture of sialylated oligosaccharides.
49. Method according to any one of embodiment 37 to 48, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal.
50. Method according to embodiment 49, wherein the cell is an Escherichia coli cell.
51. Host cell genetically modified for the production of a sialylated oligosaccharide, wherein the host cell comprises at least one nucleic acid sequence coding for an enzyme for sialylated oligosaccharide synthesis and wherein said cell is genetically modified for i) modified expression of an endogenous membrane protein, ii) expression of a homologous membrane protein, and/or iii) expression of a heterologous membrane protein,
   wherein said membrane protein comprises
   a) an amino acid sequence encoding a conserved domain GxSGxGKST (SEQ ID NO 94); and
   b) an amino acid sequence encoding a conserved domain SGGQxQRxxxxRAxxxxPK (SEQ ID NO 95), wherein x can be any amino acid.
52. Host cell according to embodiments 51, wherein said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NO 18, SEQ ID NO 15, SEQ ID NO 19 or SEQ ID NO 63.
53. Cell according to any one of the embodiments 51 to 52, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
54. Cell according to any one of the embodiments 51 to 53 wherein the cell comprises a catabolic pathway for selected mono-, di- or oligosaccharides which is at least partially inactivated, the mono-, di-, or oligosaccharides being involved in and/or required for the synthesis of sialylated oligosaccharide.
55. Cell according to any one of the embodiments 51 to 54 wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′ sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
56. Method for the production of sialylated oligosaccharide, comprising the steps of:
   a) providing a cell according to any one of the embodiments 51 to 55,
   b) culturing the cell in a medium under conditions permissive for the production of said sialylated oligosaccharide,
   c) separating said sialylated oligosaccharide from the culture.
57. Use of a membrane protein selected from the group of membrane proteins as defined in any one of the embodiments 37 to 50 in the fermentative production of sialylated oligosaccharide.
58. Use of a cell according to any one of the embodiments 51 to 55, in a method for the production of sialylated oligosaccharide.
59. Use of a cell according to any one of embodiments 57 or 58 wherein said sialylated oligosaccharide 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
60. A bacterial cell to be stably cultured in a medium for the production of oligosaccharides, said oligosaccharides being sialyllactose, the cell being transformed to comprise at least one nucleic acid sequence coding for a sialyltransferase, characterized in that: the cell in addition is transformed to comprise at least one nucleic acid sequence coding for a membrane protein wherein said membrane protein comprises a) an amino acid sequence encoding a conserved domain GxSGxGKST (SEQ ID NO 94) and b) an amino acid sequence encoding a conserved domain SGGQxQRxxxxRAxxxxPK (SEQ ID NO 95), wherein x can be any amino acid, and which protein is overexpressed.
61. The bacterial cell according to embodiment 60, characterized in that the cell is an Escherichia coli cell.
62. The bacterial cell according to any one of embodiments 60 or 61, characterized in that the membrane protein is chosen from the group comprising selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NO 18, SEQ ID NO 15, SEQ ID NO 19, or SEQ ID NO 63.
63. The bacterial cell according to any one of embodiments 60 to 62, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
64. The bacterial cell according to anyone of embodiments 60 to 63, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
65. A method for the production of oligosaccharides, said oligosaccharides being sialyllactose, the method comprising the steps of:
   a) providing a cell according to anyone of embodiments 60 to 64,
   b) culturing the cell in a medium under conditions permissive for the production of said oligosaccharides,
   c) optionally separating said oligosaccharides from the culture.
66. The method according to any one of embodiments 37 to 50, 56 or 65, characterized in that culturing is performed using a continuous flow bioreactor.
67. The method according to any one of embodiment 37 to 50, 56 or 65, characterized in that, the medium comprises substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.
68. Method according to any one of embodiment 65 to 67, wherein said sialyllactose is 3′-sialyllactose and/or 6′-sialyllactose.
69. Method for the production of sialylated oligosaccharide by a genetically modified cell, comprising the steps of:
   providing a cell capable of producing sialylated oligosaccharide, said cell comprising at least one nucleic acid sequence coding for an enzyme for sialylated oligosaccharide synthesis,
   said cell genetically modified for i) modified expression of an endogenous membrane protein, ii) expression of a homologous membrane protein, and/or iii) expression of a heterologous membrane protein, and wherein said membrane protein is an MFS transporter and comprises a) an amino acid sequence encoding a conserved domain [AGMS]x[FLMVY]x[DGKNQR]xx[EGST][PRTVY][KR]x[GILMV] (SEQ ID NO 96) and b) an amino acid sequence encoding a conserved domain [LRST]xxx[AG][AFILV] (SEQ ID NO 97), wherein x can be any distinct amino acid,
   culturing the cell in a medium under conditions permissive for the production of sialylated oligosaccharide,
   optionally separating sialylated oligosaccharide from the culture.
70. Method according to embodiment 69, wherein said modified expression in i) or expression in ii) and/or iii) is an overexpression of said membrane protein.
71. Method according to any one of embodiments 69 or 70, wherein said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122.
72. Method for the production of sialylated oligosaccharide according to any one of embodiments 69 to 71, the method further comprising at least one of the following steps:
   i) adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   ii) adding a precursor feed in a continuous manner to the culture medium over the course of 1 day, 2 days, 3 days, 4 days, 5 days by means of a feeding solution;
   iii) adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   iv) said method resulting in a sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
73. The method of embodiment 72, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.
74. The method of any one of the embodiments 72 or 73, wherein said precursor feed is accomplished by adding precursor to the cultivation medium in a concentration, such, that throughout the production phase of the cultivation a precursor concentration of at least 5 mM, preferably 10 mM or 30 mM is obtained.
75. The method of any of the embodiments 72, 73 or 74, wherein the host cells are cultivated for at least about 60, 80, 100, or about 120 hours or in a continuous manner.
76. The method of any one of embodiments 69 to 75, wherein a precursor feed is added to the culture medium and wherein said precursor is chosen from the group comprising lactose, lacto-N-biose (LN B), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.
77. The method of any one of embodiments 69 to 76, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.
78. The method of any one of embodiments 69 to 77, wherein a first phase of exponential cell growth is provided by adding a carbon-based substrate, preferably glucose or sucrose, to the culture medium before the lactose is added to the culture medium in a second phase.
79. Method according to any one of embodiments 69 to 78, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
80. Method according to any one of the embodiments 69 to 79, wherein the method is producing a mixture of sialylated oligosaccharides.
81. Method according to any one of embodiment 69 to 80, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal.
82. Method according to embodiment 81, wherein the cell is an Escherichia coli cell.
83. Host cell genetically modified for the production of a sialylated oligosaccharide, wherein the host cell comprises at least one nucleic acid sequence coding for an enzyme for sialylated oligosaccharide synthesis and wherein said cell is genetically modified for i) modified expression of an endogenous membrane protein, ii) expression of a homologous membrane protein, and/or iii) expression of a heterologous membrane protein,
   wherein said membrane protein is an MFS transporter and comprises
   a) an amino acid sequence encoding a conserved domain [AGMS]x[FLMVY]x[DGKNQR]xx[EGST][PRTVY][KR]x[GILMV] (SEQ ID NO 96) and b) an amino acid sequence encoding a conserved domain [LRST]xxx[AG][AFILV] (SEQ ID NO 97), wherein x can be any distinct amino acid.
84. Host cell according to embodiment 83, wherein said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122.
85. Cell according to any one of the embodiments 83 to 84, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
86. Cell according to any one of the embodiments 83 to 85 wherein the cell comprises a catabolic pathway for selected mono-, di- or oligosaccharides which is at least partially inactivated, the mono-, di-, or oligosaccharides being involved in and/or required for the synthesis of sialylated oligosaccharide.
87. Cell according to any one of the embodiments 83 to 86 wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
88. Method for the production of sialylated oligosaccharide, comprising the steps of:
   a) providing a cell according to any one of the embodiments 83 to 87,
   b) culturing the cell in a medium under conditions permissive for the production of said sialylated oligosaccharide,
   c) separating said sialylated oligosaccharide from the culture.
89. Use of a membrane protein selected from the group of membrane proteins as defined in any one of the embodiments 69 to 82 in the fermentative production of sialylated oligosaccharide.
90. Use of a cell according to any one of the embodiments 83 to 87, in a method for the production of sialylated oligosaccharide.
91. Use of a cell according to any one of embodiments 89 or 90 wherein said sialylated oligosaccharide 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
92. A bacterial cell to be stably cultured in a medium for the production of oligosaccharides, said oligosaccharides being sialyllactose, the cell being transformed to comprise at least one nucleic acid sequence coding for a sialyltransferase, characterized in that:
   the cell in addition is transformed to comprise at least one nucleic acid sequence coding for a membrane protein wherein said membrane protein is an MFS transporter and comprises a) an amino acid sequence encoding a conserved domain [AGMS]x[FLMVY]x[DGKNQR]xx[EGST][PRTVY][KR]x[GILMV] (SEQ ID NO 96) and b) an amino acid sequence encoding a conserved domain [LRST]xxx[AG][AFILV] (SEQ ID NO 97), wherein x can be any distinct amino acid, and which protein is overexpressed.
93. The bacterial cell according to embodiment 92, characterized in that the cell is an Escherichia coli cell.
94. The bacterial cell according to any one of embodiments 83 to 88 or 92 or 93, characterized in that the membrane protein is chosen from the group comprising selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122.
95. The bacterial cell according to any one of embodiments 83 to 88 or 92 to 94, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
96. The bacterial cell according to anyone of embodiments 83 to 88 or 92 to 95, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
97. A method for the production of oligosaccharides, said oligosaccharides being sialyllactose, the method comprising the steps of:
   a) providing a cell according to anyone of embodiments 83 to 88 or 92 to 96,
   b) culturing the cell in a medium under conditions permissive for the production of said oligosaccharides,
   c) optionally separating said oligosaccharides from the culture.
98. The method according to any one of embodiments 69 to 82, 88 or 97, characterized in that culturing is performed using a continuous flow bioreactor.
99. The method according to any one of embodiment 69 to 82, 88 or 97, characterized in that, the medium comprises substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.
100. Method according to any one of embodiment 97 to 99, wherein said sialyllactose is 3′-sialyllactose and/or 6′-sialyllactose.
101. Method for the production of sialylated oligosaccharide by a genetically modified cell, comprising the steps of:
   providing a cell capable of producing sialylated oligosaccharide, said cell comprising at least one nucleic acid sequence coding for an enzyme for sialylated oligosaccharide synthesis
   said cell genetically modified for i) modified expression of an endogenous membrane protein, ii) expression of a homologous membrane protein, and/or iii) expression of a heterologous membrane protein, and wherein said membrane protein is a Sugar Efflux Transporter,
   culturing the cell in a medium under conditions permissive for the production of sialylated oligosaccharide,
   optionally separating sialylated oligosaccharide from the culture.
102. Method according to embodiment 1, wherein said modified expression in i) or expression in ii) and/or iii) is an overexpression of said membrane protein.
103. Method according to any one of embodiments 101 or 102, wherein said membrane protein is an MFS transporter comprising the conserved domain L[FY]AxNR[HN]Y (SEQ ID NO 98), wherein x can be any distinct amino acid,
104. Method according to any one of embodiments 101 to 103, membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62.
105. Method for the production of sialylated oligosaccharide according to any one of embodiments 101 to 104, the method further comprising at least one of the following steps:
   i) Adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   ii) Adding a precursor feed in a continuous manner to the culture medium over the course of 1 day, 2 days, 3 days, 4 days, 5 days by means of a feeding solution;
   iii) Adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   iv) Said method resulting in a sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
106. The method of embodiment 105, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.
107. The method of any one of the embodiments 105 or 106, wherein said precursor feed is accomplished by adding precursor to the cultivation medium in a concentration, such, that throughout the production phase of the cultivation a precursor concentration of at least 5 mM, preferably 10 mM or 30 mM is obtained.
108. The method of any of the embodiments 105, 106 or 107, wherein the host cells are cultivated for at least about 60, 80, 100, or about 120 hours or in a continuous manner.
109. The method of any one of embodiments 101 to 108, wherein a precursor feed is added to the culture medium and wherein said precursor is chosen from the group comprising lactose, lacto-N-biose (LN B), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.
110. The method of any one of embodiments 101 to 109, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.
111. The method of any one of embodiments 101 to 110, wherein a first phase of exponential cell growth is provided by adding a carbon-based substrate, preferably glucose or sucrose, to the culture medium before the lactose is added to the culture medium in a second phase.
112. Method according to any one of embodiments 101 to 111, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
113. Method according to any one of the embodiments 101 to 112, wherein the method is producing a mixture of sialylated oligosaccharides.
114. Method according to any one of embodiment 101 to 113, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal.
115. Method according to embodiment 114, wherein the cell is an Escherichia coli cell.
116. Host cell genetically modified for the production of a sialylated oligosaccharide, wherein the host cell comprises at least one nucleic acid sequence coding for an enzyme for sialylated oligosaccharide synthesis and wherein said cell is genetically modified for i) modified expression of an endogenous membrane protein, ii) expression of a homologous membrane protein, and/or iii) expression of a heterologous membrane protein,
   wherein said membrane protein is a Sugar Efflux Transporter.
117. Cell according to embodiment 116, wherein said modified expression in i) or expression in ii) and/or iii) is an overexpression of said membrane protein.
118. Cell according to any one of embodiments 116 or 117, wherein said membrane protein is an MFS transporter comprising the conserved domain L[FY]AxNR[HN]Y (SEQ ID NO 98), wherein x can be any amino acid.
119. Host cell according to any one of embodiments 116 to 118, wherein said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62.
120. Cell according to any one of the embodiments 116 to 119, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
121. Cell according to any one of the embodiments 116 to 120 wherein the cell comprises a catabolic pathway for selected mono-, di- or oligosaccharides which is at least partially inactivated, the mono-, di-, or oligosaccharides being involved in and/or required for the synthesis of sialylated oligosaccharide.
122. Cell according to any one of the embodiments 116 to 121 wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
123. Method for the production of sialylated oligosaccharide, comprising the steps of:
   a) providing a cell according to any one of the embodiments 115 to 119,
   b) culturing the cell in a medium under conditions permissive for the production of said sialylated oligosaccharide,
   c) separating said sialylated oligosaccharide from the culture.
124. Use of a membrane protein selected from the group of membrane proteins as defined in any one of the embodiments 101 to 104 in the fermentative production of sialylated oligosaccharide.
125. Use of a cell according to any one of the embodiments 116 to 122, in a method for the production of sialylated oligosaccharide.
126. Use of a cell according to any one of embodiments 124 or 125 wherein said sialylated oligosaccharide 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose 11 (SLNH-II), disialyl-lacto-N-tetraose (DS-LNT), 6′-O-sialylated-lacto-N-neotetraose, 3′ sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
127. A bacterial cell to be stably cultured in a medium for the production of oligosaccharides, said oligosaccharides being sialyllactose, the cell being transformed to comprise at least one nucleic acid sequence coding for a sialyltransferase, characterized in that: the cell in addition is transformed to comprise at least one nucleic acid sequence coding for a protein of the Sugar Efflux Transporter (SET) family, and which protein is overexpressed.
128. The bacterial cell according to embodiment 127, characterized in that the cell is an Escherichia coli cell.
129. The bacterial cell according to any one of embodiment 127 or 128, wherein said membrane protein is an MFS transporter comprising the conserved domain L[FY]AxNR[HN]Y (SEQ ID NO 98), wherein x can be any distinct amino acid.
130. The bacterial cell according to any one of embodiments 127 to 129, characterized in that the membrane protein is chosen from the group comprising selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62.
131. The bacterial cell according to any one of embodiments 127 to 130, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
132. The bacterial cell according to anyone of embodiments 127 to 131, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
133. A method for the production of oligosaccharides, said oligosaccharides being sialyllactose, the method comprising the steps of:
   a) providing a cell according to anyone of embodiments 116 to 122, 127 to 132,
   b) culturing the cell in a medium under conditions permissive for the production of said oligosaccharides,
   c) optionally separating said oligosaccharides from the culture.
134. The method according to any one of embodiments 101 to 115, 123 or 133, characterized in that culturing is performed using a continuous flow bioreactor.
135. The method according to any one of embodiment 101 to 115, 123, 133 or 134, characterized in that, the medium comprises substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.
136. Method according to any one of embodiment 133 to 135, wherein said sialyllactose is 3′-sialyllactose and/or 6′-sialyllactose.
137. Method for the production of sialylated oligosaccharide by a genetically modified cell, comprising the steps of:
   providing a cell capable of producing sialylated oligosaccharide, said cell comprising at least one nucleic acid sequence coding for an enzyme for sialylated oligosaccharide synthesis
   said cell genetically modified for i) modified expression of an endogenous membrane protein, ii) expression of a homologous membrane protein, and/or iii) expression of a heterologous membrane protein, and wherein said membrane protein is a siderophore exporter,
   culturing the cell in a medium under conditions permissive for the production of sialylated oligosaccharide,
   optionally separating sialylated oligosaccharide from the culture.
138. Method according to embodiment 137, wherein said modified expression in i) or expression in ii) and/or iii) is an overexpression of said membrane protein
139. Method according to any one of embodiments 137 or 138, wherein said siderophore exporter is part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or
   bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP.
140. Method according to any one of embodiment 137 to 139, wherein said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122.
141. Method for the production of sialylated oligosaccharide according to any one of embodiments 137 to 140, the method further comprising at least one of the following steps:
   i) Adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   ii) Adding a precursor feed in a continuous manner to the culture medium over the course of 1 day, 2 days, 3 days, 4 days, 5 days by means of a feeding solution;
   iii) Adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   iv) Said method resulting in a sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
142. The method of embodiment 141, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.
143. The method of any one of the embodiments 141 or 142, wherein said precursor feed is accomplished by adding precursor to the cultivation medium in a concentration, such, that throughout the production phase of the cultivation a precursor concentration of at least 5 mM, preferably 10 mM or 30 mM is obtained.
144. The method of any of the embodiments 141, 142 or 143, wherein the host cells are cultivated for at least about 60, 80, 100, or about 120 hours or in a continuous manner.
145. The method of any one of embodiments 137 to 144, wherein a precursor feed is added to the culture medium and wherein precursor is chosen from the group comprising lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of, Lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.
146. The method of any one of embodiments 137 to 145, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.
147. The method of any one of embodiments 137 to 146, wherein a first phase of exponential cell growth is provided by adding a carbon-based substrate, preferably glucose or sucrose, to the culture medium before the lactose is added to the culture medium in a second phase.
148. Method according to any one of embodiments 137 to 147, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
149. Method according to any one of the embodiments 137 to 148, wherein the method is producing a mixture of sialylated oligosaccharides.
150. Method according to any one of embodiment 137 to 149, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal, preferably the cell is an Escherichia coli cell.
151. Host cell genetically modified for the production of a sialylated oligosaccharide, wherein the host cell comprises at least one nucleic acid sequence coding for an enzyme for sialylated oligosaccharide synthesis and wherein said cell is genetically modified for i) modified expression of an endogenous membrane protein, ii) expression of a homologous membrane protein, and/or iii) expression of a heterologous membrane protein,
   wherein said membrane protein is a siderophore exporter.
152. Host cell according to embodiments 151, wherein said membrane protein is part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or
   bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SF1, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP.
153. Cell according to any one of the embodiments 151 or 152, wherein said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122.
154. Cell according to any one of embodiments 151 to 153, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
155. Cell according to any one of the embodiments 151 to 154, wherein the cell comprises a catabolic pathway for selected mono-, di- or oligosaccharides which is at least partially inactivated, the mono-, di-, or oligosaccharides being involved in and/or required for the synthesis of sialylated oligosaccharide.
156. Cell according to any one of the embodiments 151 to 155, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialyl N-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
157. Method for the production of sialylated oligosaccharide, comprising the steps of:
   a) providing a cell according to any one of the embodiments 151 to 156,
   b) culturing the cell in a medium under conditions permissive for the production of said sialylated oligosaccharide,
   c) separating said sialylated oligosaccharide from the culture.
158. Use of a membrane protein selected from the group of membrane proteins as defined in any one of the embodiments 137 to 150 in the fermentative production of sialylated oligosaccharide.
159. Use of a cell according to any one of the embodiments 151 to 156, in a method for the production of sialylated oligosaccharide.
160. Use of a cell according to any one of embodiments 158 or 159, wherein said sialylated oligosaccharide 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
161. A bacterial cell to be stably cultured in a medium for the production of oligosaccharides, said oligosaccharides being sialyllactose, the cell being transformed to comprise at least one nucleic acid sequence coding for a sialyltransferase, characterized in that: the cell in addition is transformed to comprise at least one nucleic acid sequence coding for a membrane protein wherein said membrane protein is a siderophore exporter, and which protein is overexpressed.
162. The bacterial cell according to embodiment 161, characterized in that the cell is an Escherichia coli cell.
163. The bacterial cell according to any one of embodiments 161 or 162, wherein the membrane protein is part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or
   bactNOG families 05E8G, 08HFG, 089VA, 07TN1, 05C0R, 07Y9F, 05CSH, 05QRD, 05EDF, 05C6X, 08NGX, 05C2C, 07FU4, 07U9Z, 080SS, 07SF1, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP.
164. The bacterial cell according to any one of embodiments 161 to 163, wherein said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above transporter membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122.
165. The bacterial cell according to any one of embodiments 151 to 156, 161 to 164, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
166. The bacterial cell according to anyone of embodiments 151 to 146, 161 to 165, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
167. A method for the production of oligosaccharides, said oligosaccharides being sialyllactose, the method comprising the steps of:
   a) providing a cell according to anyone of embodiments 151 to 146, 161 to 166,
   b) culturing the cell in a medium under conditions permissive for the production of said oligosaccharides,
   c) optionally separating said oligosaccharides from the culture.
168. The method according to any one of embodiments 137 to 150, 157 or 167, characterized in that culturing is performed using a continuous flow bioreactor.
169. The method according to any one of embodiments 137 to 150, 157, 167 or 168, characterized in that, the medium comprises substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.
170. Method according to any one of embodiments 137 to 150, 157, 165 to 167, wherein said sialyllactose is 3′-sialyllactose and/or 6′-sialyllactose.

Moreover, the present invention relates to the following preferred specific embodiments:
1. Method for the production of sialylated oligosaccharide by a genetically modified cell, comprising the steps of:
   providing a cell capable of producing sialylated oligosaccharide, said cell comprising at least one nucleic acid sequence coding for an enzyme for sialylated oligosaccharide synthesis,
   said cell genetically modified for i) overexpression of an endogenous membrane protein, ii) expression or overexpression of a homologous membrane protein, and/or iii) expression or overexpression of a heterologous membrane protein
   culturing the cell in a medium under conditions permissive for the production of sialylated oligosaccharide
   optionally separating sialylated oligosaccharide from the culture.
2. Method according to preferred embodiment 1, wherein said cell is genetically modified for the production of sialylated oligosaccharide and wherein said genetically modified cell a) excretes sialylated oligosaccharide at a ratio of the supernatant concentration to whole broth concentration higher than 0.5 and/or b) has an enhanced production of sialylated oligosaccharide compared to a cell with the same genetic makeup but lacking the i) overexpression of the endogenous membrane protein, ii) expression or overexpression of the homologous membrane protein and/or iii) expression or overexpression of the heterologous membrane protein, respectively.
3. Method according to any one of preferred embodiment 1 or 2, wherein said membrane protein comprises
   i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TN1, 05C0R, 07Y9F, 05CSH, 05QRD, 05EDF, 05C6X, 08NGX, 05C2C, 07FU4, 07U9Z, 080SS, 07SF1, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
   vi) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
   iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or an amino acid sequence encoding a Sugar Efflux Transporter, preferably said membrane protein is an MFS transporter comprising the conserved domain L[FY]AxNR[HN]Y (SEQ ID NO: 98), wherein x can be any distinct amino acid.
4. Method according to any one of preferred embodiments 1 to 3, wherein
   i) when said membrane protein is a siderophore exporter, said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides;
   ii) when said membrane protein is an ABC transporter, said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides;
   iii) when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides;
   iv) when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.
5. Method according to any one of the previous preferred embodiments, the method further comprising at least one of the following steps:
   i) Adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m³ (cubic meter), preferably in a continuous manner, and preferably so that the final volume of the culture medium is not more than three-fold, preferably not more than two-fold, more preferably less than 2-fold of the volume of the culture medium before the addition of said precursor feed;
   ii) Adding a precursor feed in a continuous manner to the culture medium over the course of 1 day, 2 days, 3 days, 4 days, 5 days by means of a feeding solution;
   iii) Adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
   iv) Said method resulting in sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.
6. Method according to preferred embodiment 5, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.
7. Method according to any one of preferred embodiments 5 or 6, wherein said precursor feed is accomplished by adding precursor to the cultivation medium in a concentration, such, that throughout the production phase of the cultivation a precursor concentration of at least 5 mM, preferably 10 mM or 30 mM is obtained.
8. Method according to any one of preferred embodiments 5 to 7, wherein the host cells are cultivated for at least about 60, 80, 100, or about 120 hours or in a continuous manner.
9. Method according to any one of preferred embodiments 1 to 8, wherein a precursor feed is added to the culture medium and wherein precursor is chosen from the group comprising lactose, lacto-N-biose (LN B), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.
10. Method according to any one of preferred embodiments 1 to 9, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.
11. Method according to any one of preferred embodiments 1 to 10, wherein a first phase of exponential cell growth is provided by adding a carbon-based substrate, preferably glucose or sucrose, to the culture medium before the lactose is added to the culture medium in a second phase.
12. Method according to any one of preferred embodiments 1 to 11, wherein said sialylated oligosaccharide is 6′-sialyllactose, 3′-sialyllactose, 3-fucosyl-3′-sialyllactose (3′-O-sialyl-3 fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
13. Method according to any one of preferred embodiments 1 to 12, wherein the method is producing a mixture of sialylated oligosaccharides.
14. Method according to any one of preferred embodiments 1 to 13, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal, preferably the cell is an Escherichia coli cell.
15. Host cell genetically modified for the production of sialylated oligosaccharide, wherein the host cell comprises at least one nucleic acid sequence coding for an enzyme for sialylated oligosaccharide synthesis and wherein said cell is genetically modified for i) overexpression of an endogenous membrane protein, ii) expression or overexpression of a homologous membrane protein, and/or iii) expression or overexpression of a heterologous membrane protein, wherein said membrane protein comprises
   i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SF1, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
   ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
   iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
   iv) an amino acid sequence encoding a Sugar Efflux Transporter, preferably said membrane protein is an MFS transporter comprising the conserved domain L[FY]AxNR[HN]Y (SEQ ID NO: 98), wherein x can be any distinct amino acid.
16. Cell according to preferred embodiment 15, wherein
   i) when said membrane protein is a siderophore exporter, said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides;
   ii) when said membrane protein is an ABC transporter, said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides;
   iii) when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides;
   iv) when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.
17. Cell according to any one of preferred embodiments 15 or 16, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.
18. Cell according to any one of preferred embodiments 15 to 17, wherein the cell comprises a catabolic pathway for selected mono-, di- or oligosaccharides which is at least partially inactivated, the mono-, di-, or oligosaccharides being involved in and/or required for the synthesis of sialylated oligosaccharide.
19. Cell according to any one of preferred embodiments 15 to 18, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
20. Cell according to any one of preferred embodiments 15 to 19, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
21. Cell according to any one of preferred embodiments 15 to 20, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
22. Use of a membrane protein selected from the group of membrane proteins as defined in any one of the preferred embodiments 1 to 14 in the fermentative production of sialylated oligosaccharide.
23. Use of a cell according to any one of the preferred embodiments 15 to 21, in a method for the production of sialylated oligosaccharide.
24. Use of a membrane protein according to preferred embodiment 22 wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
25. Use of a cell according to preferred embodiment 23, wherein said sialylated oligosaccharide is 6′-sialyllactose, 3′-sialyl lactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′ sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.
26. A bacterial cell for the production of sialyllactose, the cell being transformed to comprise at least one nucleic acid sequence coding for a sialyltransferase, characterized in that: the cell in addition is transformed to comprise at least one nucleic acid sequence coding for a membrane protein wherein said membrane protein comprises
   i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08JJ6, 08JJA, 05FDX, 05EGG, 08JN3, 08N1B, 051D1, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
   ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
   iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
   iv) an amino acid sequence encoding a Sugar Efflux Transporter, preferably said membrane protein is an MFS transporter comprising the conserved domain L[FY]AxNR[HN]Y (SEQ ID NO: 98), wherein x can be any distinct amino acid.
27. Bacterial cell according to preferred embodiment 26, characterized in that the cell is an Escherichia coli cell.
28. Bacterial cell according to any one of preferred embodiments 26 or 27, wherein
   i) when said membrane protein is a siderophore exporter, said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides;
   ii) when said membrane protein is an ABC transporter, said membrane protein is selected from oppF from Escherichia coli K12 MG1655 with SEQ ID NO 18, ImrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides;
   iii) when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides;
   iv) when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.
29. Bacterial cell according to any one of preferred embodiments 26 to 28, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.
30. Bacterial cell according to any one of preferred embodiments 26 to 29, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.
31. A method for the production of oligosaccharides, said oligosaccharides being sialyllactose, the method comprising the steps of:
   a) providing a bacterial cell according to any one of preferred embodiments 26 to 30,
   b) culturing the cell in a medium under conditions permissive for the production of said oligosaccharides,
   c) optionally separating said oligosaccharides from the culture.
32. The method according to any one of preferred embodiments 1 to 14, 22 to 25 or 31, characterized in that culturing is performed using a continuous flow bioreactor.
33. The method according to any one of preferred embodiments 1 to 14, 22 to 25, 31 or 32, characterized in that the medium comprises substrates required for the synthesis of said oligosaccharides, wherein the substrates are selected from the group consisting of arabinose, threose, erythrose, ribose, ribulose, xylose, glucose, D-2-deoxy-2-amino-glucose, N-acetylglucosamine, glucosamine, fructose, mannose, galactose, N-acetylgalactosamine, galactosamine, sorbose, fucose, N-acetylneuraminic acid, glycoside, non-natural sugar, nucleobase, nucleoside, nucleotide and any possible di- or polymer thereof; lactose, maltose, glycerol, sucrose.
34. Method according to any one of preferred embodiments 1 to 14, 22 to 25, 31 to 33, wherein said sialyllactose is 3′-sialyllactose and/or 6′-sialyllactose.

The following drawings and examples will serve as further illustration and clarification of the present invention and are not intended to be limiting.

### DESCRIPTION OF THE FIGURES

FIG. 1: Whole broth measurement in relative percentages (%) obtained in a growth experiment with strains expressing membrane proteins with SEQ ID 02, 03, 04, 06, 07, 09, 10, 11, 14, 15, 16, or 18 in TU 01, SEQ ID 10 in TU 03 or SEQ ID 20 and 21 in their native transcriptional operon structure and all expressing a sialyllactose pathway with α2,6-sialyltransferase ST1 (SEQ ID NO 32). The growth experiment was performed in MMsf medium supplemented with 20 g/L lactose as precursor for 6′-SL. The dashed horizontal line indicates the setpoint to which all adaptations were normalized.

FIG. 2: 6′-SL export ratio in relative percentages (%) obtained in a growth experiment with strains expressing membrane proteins with SEQ ID 02, 03, 04, 06, 07, 09, 10, 11, 12, 13, 14, 15, 16, 18 or 19 in TU 01, SEQ ID 19 in TU 02, SEQ ID 10 in TU 03 or SEQ ID 20 and 21 in their native transcriptional operon structure and all expressing a sialyllactose pathway with α2,6-sialyltransferase ST1 (SEQ ID NO 32). The growth experiment was performed in MMsf medium supplemented with 20 g/L lactose as precursor for 6′-SL. The dashed horizontal line indicates the setpoint to which all adaptations were normalized.

FIG. 3: Growth speed in relative percentages (%) obtained in a growth experiment with strains expressing the membrane proteins with SEQ ID NOs 01, 02, 03, 04, 05, 06, 07, 08, 09, 10, 11, 12, 13, 16, 17 or 18 in TU 01, SEQ ID NO 19 in TU 02 or SEQ ID NOs 20 and 21 in their native transcriptional operon structure and all expressing a sialyllactose pathway with α2,6-sialyltransferase ST1 (SEQ ID NO 32). The growth experiment was performed in MMsf medium supplemented with 20 g/L lactose as precursor for 6′-SL. The dashed horizontal line indicates the setpoint to which all adaptations were normalized.

FIG. 4: 6′-SL export ratio in relative percentages (%) obtained in a growth experiment with a strain expressing the membrane protein with SEQ ID 02, 04, 07, 09, 11, 16 or 18 in TU 01 or SEQ ID 20 and 21 in their native transcriptional operon structure and all expressing a sialyllactose pathway with α2,6-sialyltransferase ST1 (SEQ ID NO 32). All genes were integrated into the genome. The growth experiment was performed in MMsf medium supplemented with 20 g/L lactose as precursor for 6′-SL. The dashed horizontal line indicates the setpoint to which all adaptations were normalized.

FIG. 5: Whole broth measurement in relative percentages (%) obtained in a growth experiment with the strain expressing the membrane protein with SEQ ID 09 in the different transcriptional units TU 04 up to TU 12 from the host's genome and expressing a sialyllactose pathway with α2,6-sialyltransferase ST1 (SEQ ID NO 32). All genes were integrated into the genome. The growth experiment was performed in MMsf medium supplemented with 20 g/L lactose as precursor for 6′-SL. The dashed horizontal line indicates the setpoint to which all adaptations were normalized.

FIG. 6: 6′-SL export ratio in relative percentages (%) obtained in a growth experiment with strains expressing membrane proteins with SEQ ID 09 in the different transcriptional units TU 04 up to TU 12 from the host's genome and expressing a sialyllactose pathway with α2,6-sialyltransferase ST1 (SEQ ID NO 32). All genes were integrated into the genome. The growth experiment was performed in MMsf medium supplemented with 20 g/L lactose as precursor for 6′-SL. The dashed horizontal line indicates the setpoint to which all adaptations were normalized.

FIG. 7: 6′-SL export ratio in relative percentages (%) obtained from samples taken during four different fermentation runs expressing membrane protein EcEntS with SEQ ID 09 in TU 01 and expressing a sialyllactose pathway with α2,6-sialyl transferase ST1 (SEQ ID NO 32) on the genome. For Ferm 03, an additional sialyltransferase was expressed from a p15A plasmid. The fermentations were performed in minimal medium for fermentations supplemented with 100 g/L lactose as precursor for 6′-SL. The dashed horizontal line indicates the setpoint to which all adaptations were normalized.

FIG. 8: Whole broth measurement of 6′-SL in relative percentages (%) obtained in a growth experiment with the strains expressing a membrane protein with SEQ ID NO 19 in TU 02, SEQ ID NOs 66 or 68 in TU08, SEQ ID NOs 19 or 99 in TU 13, SEQ ID NOs 100, 19, 57, 60 or 74 in TU 14, SEQ ID NOs 102, 103, 105, 106, 108, 109, 110, 111, 114, 115, 117, 118, 119 or 121 in TU 15, SEQ ID NO 66 in TU 16, SEQ ID NO 71 in TU 17, SEQ ID NOs 47, 55 or 75 in TU 18, SEQ ID NOs 19 or 68 in TU 21, SEQ ID NO 80 in TU 22, SEQ ID NOs 70, 71, 72, 74 or 80 in TU 25, SEQ ID NOs 75 or 81 in TU 26 or SEQ ID NO 80 in TU 27 from plasmid and expressing a sialyllactose pathway with α2,6-sialyltransferase ST1 (SEQ ID NO 32). The growth experiment was performed in MMsf medium supplemented with 20 g/L lactose as precursor for 6′-SL. The dashed horizontal line indicates the setpoint to which all adaptations were normalized.

FIG. 9: 6′-SL export ratio in relative percentages (%) obtained in a growth experiment with strains expressing a membrane protein with SEQ ID NO 66 in TU 01, SEQ ID NO 19 in TU 02, SEQ ID NOs 19, 66, 67, 68 or 99 in TU 08, SEQ ID NOs 19, 66, 67 or 99 in TU 13, SEQ ID NOs 100, 19, 57, 59 or 74 in TU 14, SEQ ID NOs 102, 103, 104, 105, 106, 108, 109, 110, 111, 112, 113, 114, 115, 116, 117, 118, 119, 120, 121 or 122 in TU 15, SEQ ID NOs 19 or 66 in TU 16, SEQ ID NOs 66 or 72 in TU 17, SEQ ID NOs 67, 74 or 75 in TU 18, SEQ ID NOs 19 or 67 in TU 19 and TU 20, SEQ ID NOs 19, 67 or 68 in TU 21, SEQ ID NOs 19, 68, 79 or 80 in TU 22, SEQ ID NO 19 in TU 23, SEQ ID NO 68 in TU 24, SEQ ID NOs 71, 72, 74, 79 or 80 in TU 25, SEQ ID NOs 75, 78 or 81 in TU 26, SEQ ID NOs 72 or 80 in TU 27 or SEQ ID NO 68 in TU 29 from plasmid and expressing a sialyllactose pathway with α2,6-sialyl transferase ST1 (SEQ ID NO 32). The growth experiment was performed in MMsf medium supplemented with 20 g/L lactose as precursor for 6′-SL. The dashed horizontal line indicates the setpoint to which all adaptations were normalized.

FIG. 10: Growth speed in relative percentages (%) obtained in a growth experiment with strains expressing a membrane protein with SEQ ID NO 66 in TU 01, SEQ ID NO 19 in TU 07, SEQ ID NOs 19, 66, 67 or 99 in TU 08 and TU 13, SEQ ID NOs 100, 19, 48, 57, 59, 60 or 74 in TU 14, SEQ ID NOs 102, 103, 104, 105, 106, 107, 108, 109, 110, 111, 112, 113, 114, 115, 116, 117, 118, 119 or 121 in TU 15, SEQ ID NOs 19 or 66 in TU 16, SEQ ID NOs 66, 71 or 72 in TU 17, SEQ ID NOs 47, 55 or 67 in TU 18, SEQ ID NOs 19 or 67 in TU 19 and TU 20, SEQ ID NOs 19 or 68 in TU 21, SEQ ID NOs 19, 68 or 80 in TU 22, SEQ ID NO 19 in TU 23, SEQ ID NO 68 in TU 24, SEQ ID NOs 71, 72, 74 or 80 in TU 25, SEQ ID NOs 75 or 78 in TU 26, SEQ ID NO 80 in TU 27 or SEQ ID NO 101 in TU 28 from plasmid and expressing a sialyllactose pathway with α2,6-sialyl transferase ST1 (SEQ ID NO 32). The growth experiment was performed in MMsf medium supplemented with 20 g/L lactose as precursor for 6′-SL. The dashed horizontal line indicates the setpoint to which all adaptations were normalized.

FIG. 11: Whole broth measurement of 3′-SL in relative percentages (%) obtained in a growth experiment with the strains expressing a membrane protein with SEQ ID NOs 02, 07, 11, 14, 16 or 18 in TU 01 or SEQ ID NOs 20 or 21 in their natural operon structure from plasmid and expressing a sialyllactose pathway with α2,3-sialyl transferase ST2 (SEQ ID NO 33). The growth experiment was performed in MMsf medium supplemented with 20 g/L lactose as precursor for 3′-SL. The dashed horizontal line indicates the setpoint to which all adaptations were normalized.

FIG. 12: 3′-SL export ratio in relative percentages (%) obtained in a growth experiment with strains expressing a membrane protein with SEQ ID NOs 02, 07, 09, 11, 14, 16 or 18 in TU 01 or SEQ ID NOs 20 or 21 in their natural operon structure from plasmid and expressing a sialyllactose pathway with α2,3-sialyl transferase ST2 (SEQ ID NO 33). The growth experiment was performed in MMsf medium supplemented with 20 g/L lactose as precursor for 3′-SL. The dashed horizontal line indicates the setpoint to which all adaptations were normalized.

### EXAMPLES

### Example 1: Material and Methods

Material and Methods Escherichia coli

### Media

Three different media were used, namely a rich Luria Broth (LB), a minimal medium for shake flask (MMsf) and a minimal medium for fermentation (MMf). Both minimal media use a trace element mix.

Trace element mix consisted of 3.6 g/L FeCl₂·4H₂O, 5 g/L CaCl₂·2H₂O, 1.3 g/L MnCl₂·2H₂O, 0.38 g/L CuCl₂·2H₂O, 0.5 g/L CoCl₂·6H₂O, 0.94 g/L ZnCl₂, 0.0311 g/L H₃BO₄, 0.4 g/L Na₂EDTA·2H₂O and 1.01 g/L thiamine·HCl. The molybdate solution contained 0.967 g/L NaMoO₄·2H₂O. The selenium solution contained 42 g/L SeO₂.

The Luria Broth (LB) medium consisted of 1% tryptone peptone (Difco, Erembodegem, Belgium), 0.5% yeast extract (Difco) and 0.5% sodium chloride (VWR, Leuven, Belgium). Luria Broth agar (LBA) plates consisted of the LB media, with 12 g/L agar (Difco, Erembodegem, Belgium) added.

The minimal medium for the shake flasks (MMsf) experiments contained 2.00 g/L NH₄Cl, 5.00 g/L (NH₄)₂SO₄, 2.993 g/L KH₂PO₄, 7.315 g/L K₂H PO₄, 8.372 g/L MOPS, 0.5 g/L NaCl, 0.5 g/L MgSO₄.7H₂O, 14.26 g/L sucrose or another carbon source when specified in the examples, 1 ml/L trace element mix, 100 μl/L molybdate solution, and 1 mL/L selenium solution. The medium was set to a pH of 7 with 1M KOH. Depending on the experiment lactose, LNB or LacNAc could be added as a precursor.

The minimal medium for fermentations (MMf) contained 6.75 g/L NH₄Cl, 1.25 g/L (NH₄)₂SO₄, 2.93 g/L KH₂PO₄ and 7.31 g/L KH₂PO₄, 0.5 g/L NaCl, 0.5 g/L MgSO₄·7H2O, 14.26 g/L sucrose, 1 mL/L trace element mix, 100 μL/L molybdate solution, and 1 mL/L selenium solution with the same composition as described above.

Complex medium, e.g. LB, was sterilized by autoclaving (121° C., 21′) and minimal medium by filtration (0.22 μm Sartorius). When necessary, the medium was made selective by adding an antibiotic (e.g. ampicillin (100 mg/L), chloramphenicol (20 mg/L), carbenicillin (100 mg/L), spectinomycin (40 mg/L) and/or kanamycin (50 mg/L)).

### Plasmids

pKD46 (Red helper plasmid, Ampicillin resistance), pKD3 (contains an FRT-flanked chloramphenicol resistance (cat) gene), pKD4 (contains an FRT-flanked kanamycin resistance (kan) gene), and pCP20 (expresses FLP recombinase activity) plasmids were obtained from Prof. R. Cunin (Vrije Universiteit Brussel, Belgium in 2007).

Plasmids for membrane protein and for additional sialyltransferase expression from plasmid, were constructed in a pSC101 or a p15A ori containing backbone vector, respectively, using Golden Gate assembly. All membrane protein and sialyltransferase encoding genes were synthetically synthetized at Twist Biosciences (San Francisco, USA). Polynucleotide sequences of the membrane proteins and the corresponding membrane protein polypeptides are shown in SEQ ID NOs 01 to 21, 37 to 93 and 99 to 122 and enlisted in Table 1.

### Transcription Units

Both membrane protein and sialyl transferase genes were expressed in different transcriptional units (TUs) using specific promoter, UTR and terminator combinations as enlisted in Table 2. The genes were expressed using promoters from Mutalik et al. (Nat. Methods 2013, No. 10, 354-360), as described herein as “PROM0005”, “PROM0010”, “PROM0012”, “PROM0025”, “PROM0032” and “PROM0050”, a promoter from De Mey et al. (BMC Biotechnology 2007, 7:34)), as described herein as “PROM0015” and a modified promoter of apFAB115 (as described by Mutalik et al. (Nat. Methods 2013, No. 10, 354-360), described herein as “PROM0171”. UTRs used as described herein as “UTR0003”, “UTR0011”, “UTR0013”, “UTR0014”, “UTR0029”, “UTR0038”, “UTR0051” and “UTR0055” were obtained from Mutalik et al. (Nat. Methods 2013, No. 10, 354-360). Terminators used in the examples are described as “TER0010” and “TER0020” as obtained from Dunn et al. (Nucleic Acids Res. 1980, 8(10), 2119-32) and “TER0002” are as obtained from Orosz et al. (Eur. J. Biochem. 1991, 201, 653-59). Table 2 shows the overview of the transcriptional units used in the examples by combination of the above promoter UTRs and terminators. Expression could be further facilitated by optimizing the codon usage to the codon usage of the expression host. Genes were optimized using the tools of the supplier.

TABLE 1

| | | | | Country of origin of |
|---|---|---|---|---|
| SEQ ID NO | Name/ | | | digital sequence |
| (protein) | TCDB group | Organism | Origin | information |
| 01 | EcSetA | E. coli K12 MG1655 | Synthetic | USA |
| 02 | EcSetB | E. coli K12 MG1655 | Synthetic | USA |
| 03 | EcSetC | E. coli K12 MG1655 | Synthetic | USA |
| 04 | EcMdfA | E. coli K12 MG1655 | Synthetic | USA |
| 05 | EcYnfM | E. coli K12 MG1655 | Synthetic | USA |
| 06 | EcIceT | E. coli K12 MG1655 | Synthetic | USA |
| 07 | EcYhhs | E. coli K12 MG1655 | Synthetic | USA |
| 08 | EcYdhC | E. coli K12 MG1655 | Synthetic | USA |
| 09 | EcEntS/EcYbdA | E. coli K12 MG1655 | Synthetic | USA |
| 10 | EcMhpT | E. coli K12 MG1655 | Synthetic | USA |
| 11 | EcYebQ | E. coli K12 MG1655 | Synthetic | USA |
| 12 | EcYjhB | E. coli K12 MG1655 | Synthetic | USA |
| 13 | EcBcr | E. coli K12 MG1655 | Synthetic | USA |
| 14 | EcFucP | E. coli K12 MG1655 | Synthetic | USA |
| 15 | LlImrA | Lactococcus lactis strain | Synthetic | South Korea |
| | | SRCM103457 | | |
| 16 | PcSetA_01 | Pectobacterium carotovorum | Synthetic | South Korea |
| | | (short version) | | |
| 17 | PcSetA_02 | Pectobacterium carotovorum | Synthetic | South Korea |
| | | (long version) | | |
| 18 | EcOppF | E. coli strain K12 (MG1655) | Synthetic | USA |
| 19 | Blon_2475 | B. longum subsp. Infantis | Synthetic | Germany |
| | | (strain ATCC 15697) | | |
| 20 | Blon_0247 | B. longum subsp. Infantis | Synthetic | Germany |
| | | (strain ATCC 15697) | | |
| 21 | Blon_0245 | B. longum subsp. Infantis | Synthetic | Germany |
| | | (strain ATCC 15697) | | |
| 37 | CuNAm | Curtobacterium sp. | Synthetic | USA |
| | | 314Chir4.1 | | |
| 38 | mdtD | Citrobacter freundii MGH152 | Synthetic | USA |
| 39 | mdtD | Citrobacter werkmanii NBRC | Synthetic | Belgium |
| | | 105721 | | |
| 40 | mdtD | Citrobacter amalonaticus | Synthetic | USA |
| 41 | mdtD | Klebsiella oxytoca | Synthetic | United Kingdom |
| 42 | mdtD | Escherichia albertii B156 | Synthetic | USA |
| 43 | yegB | Salmonella enterica subsp. | Synthetic | United Kingdom |
| | | salamae | | |
| 44 | mdtD | Klebsiella pneumoniae | Synthetic | USA |
| | | 30684/NJST258_2 | | |
| 45 | mdtD | Klebsiella pneumoniae kneu | Synthetic | USA |
| 46 | mdtD | Pseudocitrobacter faecalis | Synthetic | USA |
| 47 | YrMdfA | Yokenella regensburgei | Synthetic | United Kingdom |
| | | ATCC43003 | | |
| 48 | CmMdfA | Cronobacter muytjensii | Synthetic | USA |
| 49 | KoMdfA | Klebsiella oxytoca | Synthetic | Sweden |
| 50 | CkMdfA | Citrobacter koseri | Synthetic | USA |
| 51 | EmMdfA | Escherichia marmotae | Synthetic | USA |
| 52 | Cmr | Shigella flexneri | Synthetic | United Kingdom |
| 53 | SeMdfA | Salmonella enterica subsp. | Synthetic | Denmark |
| | | salamae | | |
| 54 | Cmr | Citrobacter youngae ATCC | Synthetic | USA |
| | | 29220 | | |
| 55 | CfMdfA | Citrobacter freundii | Synthetic | USA |
| 56 | EkMdfA | Enterobacter kobei | Synthetic | USA |
| 57 | EnMdfA | Enterobacter sp. | Synthetic | Australia |
| 58 | MdfA | Lelliottia sp. WB101 | Synthetic | USA |
| 59 | ElMdfA | Enterobacter ludwigii | Synthetic | USA |
| | | EcWSU1 | | |
| 60 | EsMdfA | Enterobacter soli LF7a | Synthetic | Unknown |
| 61 | TIC76629 | Bifidobacterium longum | Synthetic | USA |
| | | subsp. infantis (strain Bi-26) | | |
| 62 | CnSetA | Cedecea neteri | Synthetic | USA |
| 63 | EcgsiA | E. coli K12 MG1655 | Synthetic | USA |
| 64 | EcGalP | E. coli K12 MG1655 | Synthetic | USA |
| 65 | EcYdeA | E. coli K12 MG1655 | Synthetic | USA |
| 66 | EcWzxE | E. coli K12 MG1655 | Synthetic | USA |
| 67 | HpWzk | Helicobacter pylori strain | Synthetic | United Kingdom |
| | | ATCC 700392/26695 | | |
| 68 | Blon0345 | B. longum subsp. Infantis | Synthetic | Germany |
| | | (strain ATCC 15697) | | |
| 69 | EcMsbA | E. coli K12 MG1655 | Synthetic | USA |
| 70 | NcCDT2 | Neurospora crassa OR74A | Synthetic | USA |
| 71 | AoCDT2 | Aspergillus oryzae RIB40 | Synthetic | Japan |
| 72 | ChWzx | Chitinophaga sp. CF118 | Synthetic | USA |
| 73 | EuWzx | Eubacterium sp. CAG: 581 | Synthetic | Denmark |
| 74 | LrWzx | Lactococcus raffinolactis | Synthetic | South Korea |
| | | KACC 13441 | | |
| 75 | PrWzx | Prevotella ruminicola (AR32) | Synthetic | USA |
| 76 | PsTolC | Candidatus Planktophila | Synthetic | Switzerland |
| | | sulfonica | | |
| 77 | BhTolC | Butyrivibrio hungatei | Synthetic | USA |
| | | XBD2006 | | |
| 78 | RiMsbA | Roseburia intestinalis | Synthetic | Denmark |
| | | CAG: 13 | | |
| 79 | PgMsbA | Pedobacter ginsengisoli | Synthetic | South Korea |
| 80 | VbMsbA | Verrucomicrobia bacterium | Synthetic | USA |
| | | CG1_02_43_26 | | |
| 81 | AsNAm | Actinobaculum suis DSM | Synthetic | Unknown |
| | | 20639 | | |
| 82 | RgNAm | Ruminococcus gnavus | Synthetic | USA |
| 83 | EcmdIA | E. coli K12 MG1655 | Synthetic | USA |
| 84 | EcmdIB | E. coli K12 MG1655 | Synthetic | USA |
| 85 | EcgsiC | E. coli K12 MG1655 | Synthetic | USA |
| 86 | EcgsiD | E. coli K12 MG1655 | Synthetic | USA |
| 87 | EcoppB | E. coli K12 MG1655 | Synthetic | USA |
| 88 | EcoppC | E. coli K12 MG1655 | Synthetic | USA |
| 89 | EcoppD | E. coli K12 MG1655 | Synthetic | USA |
| 90 | EcwzxC | E. coli K12 MG1655 | Synthetic | USA |
| 91 | EcyihP | E. coli K12 MG1655 | Synthetic | USA |
| 92 | Abaet | Azospirillum brasiliense LMG | Synthetic | Belgium |
| | | 04375 | | |
| 93 | Bjnodj | Bradyrhizobium japonicum | Synthetic | USA |
| | | USDA 110 | | |
| 99 | Blon2331 | Bifidobacterium longum | Synthetic | Germany |
| | | subsp. Infantis (strain ATCC | | |
| | | 15697) | | |
| 100 | KpIceT | Klebsiella pneumoniae | Synthetic | USA |
| | | MB369 | | |
| 101 | Abaet | Azospirillum brasiliense LMG | Synthetio | Belgium |
| | | 04375 | | |
| 102 | EcAcrB | Escherichia coli str. K-12 | Synthetic | USA |
| | | substr. W3110 | | |
| 103 | EcAmpG | Escherichia coli str. K-12 | Synthetic | USA |
| | | substr. W3110 | | |
| 104 | EcEmrB | Escherichia coli str. K-12 | Synthetic | USA |
| | | substr. W3110 | | |
| 105 | EcEmrY | Escherichia coli str. K-12 | Synthetic | USA |
| | | substr. W3110 | | |
| 106 | EcEntS | Escherichia coli | Synthetic | United Kingdom |
| 107 | EcHsrA | Escherichia coli str. K-12 | Synthetic | USA |
| | | substr. W3110 | | |
| 108 | EcLgoT | Escherichia coli str. K-12 | Synthetic | USA |
| | | substr. W3110 | | |
| 109 | EcMelB | Escherichia coli str. K-12 | Synthetic | USA |
| | | substr. W3110 | | |
| 110 | EcProP | Escherichia coli str. K-12 | Synthetic | USA |
| | | substr. W3110 | | |
| 111 | EcShiA | Escherichia coli str. K-12 | Synthetic | USA |
| | | substr. W3110 | | |
| 112 | EcUidB | Escherichia coli str. K-12 | Synthetic | USA |
| | | substr. W3110 | | |
| 113 | EcYdfJ | Escherichia coli str. K-12 | Synthetic | USA |
| | | substr. W3110 | | |
| 114 | EcYhjE | Escherichia coli str. K-12 | Synthetic | USA |
| | | substr. W3110 | | |
| 115 | EcYicJ | Escherichia coli str. K-12 | Synthetic | USA |
| | | substr. W3110 | | |
| 116 | FtFsIB | Francisella tularensis subsp. | Synthetic | USA |
| | | tularensis SCHU S4 | | |
| 117 | KaEntS | Kluyvera ascorbata | Synthetic | USA |
| 118 | KiEntS | Kluyvera intermedia strain | Synthetic | USA |
| | | CAV1151 | | |
| 119 | LaEntS | Leclercia adecarboxylata | Synthetic | USA |
| | | strain USDA-ARS-USMARC- | | |
| | | 60222 | | |
| 120 | SaSfaA | Staphylococcus aureus | Synthetic | United Kingdom |
| | | subsp. aureus NCTC 8325 | | |
| 121 | SeEntS | Salmonella enterica subsp. | Synthetic | USA |
| | | arizonae | | |
| 122 | SeEntS | Salmonella enterica subsp. | Synthetic | USA |
| | | enterica serovar Derby | | |

TABLE 2

| TU number | Promoter part | UTR part | Terminator part |
|---|---|---|---|
| TU 01 | PROM0015 | UTR0003 | TER0010 |
| TU 02 | PROM0005 | UTR0014 | TER0010 |
| TU 03 | PROM0050 | UTR0014 | TER0002 |
| TU 04 | PROM0012 | UTR0003 | TER0010 |
| TU 05 | PROM0171 | UTR0003 | TER0010 |
| TU 06 | PROM0015 | UTR0014 | TER0010 |
| TU 07 | PROM0005 | UTR0014 | TER0010 |
| TU 08 | PROM0012 | UTR0014 | TER0010 |
| TU 09 | PROM0171 | UTR0014 | TER0010 |
| TU 10 | PROM0012 | UTR0029 | TER0010 |
| TU 11 | PROM0171 | UTR0029 | TER0010 |
| TU 12 | PROM0015 | UTR0051 | TER0010 |
| TU 13 | PROM0032 | UTR0014 | TER0010 |
| TU 14 | PROM0005 | UTR0055 | TER0010 |
| TU 15 | PROM0025 | UTR0014 | TER0010 |
| TU 16 | PROM0012 | UTR0055 | TER0010 |
| TU 17 | PROM0005 | UTR0014 | TER0020 |
| TU 18 | PROM0005 | UTR0038 | TER0010 |
| TU 19 | PROM0032 | UTR0038 | TER0010 |
| TU 20 | PROM0032 | UTR0055 | TER0010 |
| TU 21 | PROM0012 | UTR0038 | TER0010 |
| TU 22 | PROM0005 | UTR0038 | TER0020 |
| TU 23 | PROM0015 | UTR0003 | TER0020 |
| TU 24 | PROM0032 | UTR0014 | TER0020 |
| TU 25 | PROM0005 | UTR0011 | TER0010 |
| TU 26 | PROM0005 | UTR0011 | TER0020 |
| TU 27 | PROM0005 | UTR0055 | TER0020 |
| TU 28 | PROM0010 | UTR0013 | TER0010 |
| TU 29 | PROM0032 | UTR0038 | TER0020 |

### Strains and Mutations

Escherichia coli KU MG1655 [lambda⁻, F⁻, rph-1] was obtained from the Coli Genetic Stock Center (US), CGSC Strain #: 7740, in March 2007. Gene disruptions as well as gene introductions were performed using the technique published by Datsenko and Wanner (PNAS 97 (2000), 6640-6645). This technique is based on antibiotic selection after homologous recombination performed by lambda Red recombinase. Subsequent catalysis of a flippase recombinase ensures removal of the antibiotic selection cassette in the final production strain.

Transformants carrying a Red helper plasmid pKD46 were grown in 10 ml LB media with ampicillin, (100 mg/L) and L-arabinose (10 mM) at 30° C. to an OD_{600 nm} of 0.6. The cells were made electrocompetent by washing them with 50 ml of ice-cold water, a first time, and with 1 ml ice cold water, a second time. Then, the cells were resuspended in 50 μl of ice-cold water. Electroporation was done with 50 μl of cells and 10-100 ng of linear double-stranded-DNA product by using a Gene Pulser™ (BioRad) (600Ω, 25 μFD, and 250 volts).

After electroporation, cells were added to 1 ml LB media incubated 1 h at 37° C., and finally spread onto LB-agar containing 25 mg/L of chloramphenicol or 50 mg/L of kanamycin to select antibiotic resistant transformants. The selected mutants were verified by PCR with primers upstream and downstream of the modified region and were grown in LB-agar at 42° C. for the loss of the helper plasmid. The mutants were tested for ampicillin sensitivity.

The linear ds-DNA amplicons were obtained by PCR using pKD3, pKD4 and their derivates as template. The primers used had a part of the sequence complementary to the template and another part complementary to the side on the chromosomal DNA where the recombination must take place. For the genomic knock-out, the region of homology was designed 50-nt upstream and 50-nt downstream of the start and stop codon of the gene of interest. For the genomic knock-in, the transcriptional starting point (+1) had to be respected. PCR products were PCR-purified, digested with Dpnl, repurified from an agarose gel, and suspended in elution buffer (5 mM Tris, pH 8.0).

The selected mutants (chloramphenicol or kanamycin resistant) were transformed with pCP20 plasmid, which is an ampicillin and chloramphenicol resistant plasmid that shows temperature-sensitive replication and thermal induction of FLP synthesis. The ampicillin-resistant transformants were selected at 30° C., after which a few were colony purified in LB at 42° C. and then tested for loss of all antibiotic resistance and of the FLP helper plasmid. The gene knock-outs and knock-ins are checked with control primers (Fw/Rv-gene-out).

A sialic acid producing base strain derived from E. coli KU MG1655 was created by knocking out the genes asl, IdhA, poxB, atpl-gidB and ackA-pta, and knocking out the operons lacZYA, nagAB and the genes nanA, nanE and nanK. Additionally, the E. coli lacY gene was introduced at the location of lacZYA. A fructose kinase gene (frk) originating from Zymomonas mobilis, an E. coli W sucrose transporter (cscB), a sucrose phosphorylase (SP) originating from Bifidobacterium adolescentis, an E. coli mutant fructose-6-P-aminotransferase (EcglmS*54, as described by Deng et al. (Biochimie 88, 419-29 (2006)), glucosamine-6-P-aminotransferase from Saccharomyces cerevisiae (ScGNA1), an N-acetylglucosamine-2-epimerase from Bacteroides ovatus (BoAGE) and a sialic acid synthase from Campylobacter jejuni (CjneuB) were knocked in into the genome.

To allow production of 6′-SL, the sialic acid base strain was further modified by introducing two constructs both expressing a CMP-sialic acid synthetase from Neisseria meningitidis (NmneuA, SEQ ID NO 31) and an α-2,6-sialyltransferase from Photobacterium damselae (PdbST, SEQ ID NO 32) into the genome.

To allow production of 3′-SL, the sialic acid base strain was further modified by introducing a construct expressing a CMP-sialic acid synthetase from Neisseria meningitidis (NmneuA, SEQ ID NO 31) and an α-2,3-sialyltransferase from Neisseria meningitidis (NmST, SEQ ID NO 33) which were knocked in into the genome.

To allow production of sialylated LacNAc (sLacNAc), the sialic acid base strain was further modified by a CMP-sialic acid synthetase from Neisseria meningitidis (NmneuA, SEQ ID NO 31) and a sialyltransferase which were knocked in into the genome. For 6′-sLacNAc, a sialyltransferase from Photobacterium damselae (PdbST, SEQ ID NO 32) was used and for 3′-sLacNAc, a sialyltransferase from Neisseria meningitidis (NmST, SEQ ID NO 33) was used.

To allow production of sialylated LNB (sLNB), the sialic acid base strain was further modified by introducing a CMP-sialic acid synthetase from Neisseria meningitidis (NmneuA, SEQ ID NO 31) and a sialyltransferase which were knocked in into the genome. For 6′-sLNB, a sialyltransferase from Photobacterium damselae (PdbST, SEQ ID NO 32) was used and for 3′-sLNB, a sialyltransferase from Neisseria meningitidis (NmST, SEQ ID NO 33) was used.

To allow production of LSTa and b, the sialic acid base strain was further modified by introducing a beta-1,3-GlcNAc transferase from Neisseria meningitidis (NmIgtA, SEQ ID NO 34), a beta-1,3-galactosyltransferase from E. coli 055:H7 (EcwbgO, SEQ ID NO 36), a CMP-sialic acid synthetase and an alpha-2,3-sialyltransferase or an alpha-2,6-sialyltransferase for production of LSTa or LSTb, respectively. Alternatively, sialic acid can be fed to an optimized lacto-N-tetraose producing strain with expression of a beta-1,3-GlcNAc transferase from Neisseria meningitidis (NmIgtA, SEQ ID NO 34) and a beta-1,3-galactosyltransferase from E. coli O55:H7 (EcwbgO, SEQ ID NO 36) (as described and demonstrated in Example 8 of WO18122225), and additional expression of a CMP-sialic acid synthetase and an α-2,3-sialyltransferase or an α-2,6-sialyltransferase to allow LSTa or LSTb production, respectively.

To allow production of LSTc and d, the sialic acid base strain was further modified by introducing a beta-1,3-GlcNAc transferase from Neisseria meningitidis (NmIgtA), a beta-1,4-galactosyltransferase from Neisseria meningitidis (NmIgtB), a CMP-sialic acid synthetase and an alpha-2,3-sialyltransferase or an alpha-2,6-sialyltransferase for production of LSTc or LSTd, respectively. Alternatively, sialic acid can be fed to an optimized lacto-N-neotetraose producing strain with expression of a beta-1,3-GlcNAc transferase from Neisseria meningitidis (NmIgtA) and a beta-1,4-galactosyltransferase from Neisseria meningitidis (NmIgtB) (as described and demonstrated in Example 8 of WO18122225), and additional expression of a CMP-sialic acid synthetase and an alpha-2,3-sialyltransferase or an alpha-2,6-sialyltransferase to allow LSTc or LSTd production, respectively.

All these genes were constitutively expressed with promoters originating from the promoter library described by De Mey et al. (BMC Biotechnology, 2007) or by Mutalik et al. (Nat. Methods 2013, No. 10, 354-360). UTRs originated from Mutalik et al. (Nat. Methods 2013, No. 10, 354-360) and terminators originated from Dunn et al. (Nucleic Acids Res. 1980, 8(10), 2119-32) and Orosz et al. (Eur. J. Biochem. 1991, 201, 653-59). These genetic modifications are also described in WO18122225.

For all of the above-mentioned strains, daughter strains could further be made by adding an additional production plasmid expressing a CMP-sialic acid synthetase and an alpha-2,6- or alpha-2,3-sialyltransferase.

All membrane protein genes were evaluated in these mutant strains derived from E. coli K12 MG1655. Membrane protein genes were evaluated by either genomic or plasmid-based expression.

All strains are stored in cryovials at −80° C. (overnight LB culture mixed in a 1:1 ratio with 70% glycerol).

### Cultivation Conditions

A preculture of 96-well microtiter plate experiments was started from a cryovial, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or the whole broth concentration (by boiling the culture broth for 15 min at 60° C. before spinning down the cells (=intra- and extracellular sugar concentrations together)).

Also, a dilution of the cultures was made to measure the optical density at 600 nm. The cell performance index or CPI is determined by dividing the sialylated oligosaccharide concentrations, e.g. sialyllactose concentrations, measured in the whole broth by the biomass, in relative percentages compared to the reference strain. The biomass is empirically determined to be approximately ⅓^{rd} of the optical density measured at 600 nm. The sialylated oligosaccharide export ratio was determined by dividing the sialylated oligosaccharide concentrations measured in the supernatant by the sialylated oligosaccharide concentrations measured in the whole broth, in relative percentages compared to the reference strain.

A preculture for the bioreactor was started from an entire 1 mL cryovial of a certain strain, inoculated in 250 mL or 500 mL of MMsf medium in a 1 L or 2.5 L shake flask and incubated for 24 h at 37° C. on an orbital shaker at 200 rpm. A 5 L bioreactor was then inoculated (250 mL inoculum in 2 L batch medium); the process was controlled by MFCS control software (Sartorius Stedim Biotech, Melsungen, Germany). Culturing condition were set to 37° C., and maximal stirring; pressure gas flow rates were dependent on the strain and bioreactor. The pH was controlled at 6.8 using 0.5 M H₂SO₄ and 20% NH₄OH. The exhaust gas was cooled. 10% solution of silicone antifoaming agent was added when foaming raised during the fermentation.

Material and Methods Bacillus subtilis

### Media

Two different media are used, namely a rich Luria Broth (LB) and a minimal medium for shake flask (MMsf). The minimal medium uses a trace element mix.

Trace element mix consisted of 0.735 g/L CaCl2·2H2O, 0.1 g/L MnC12·2H2O, 0.033 g/L CuC12·2H2O, 0.06 g/L CoCl2·6H2O, 0.17 g/L ZnCl2, 0.0311 g/L H3BO4, 0.4 g/L Na2EDTA·2H2O and 0.06 g/L Na2MoO4. The Fe-citrate solution contained 0.135 g/L FeCl3·6H2O, 1 g/L Na-citrate (Hoch 1973 PMC1212887).

The Luria Broth (LB) medium consisted of 1% tryptone peptone (Difco, Erembodegem, Belgium), 0.5% yeast extract (Difco) and 0.5% sodium chloride (VWR, Leuven, Belgium). Luria Broth agar (LBA) plates consisted of the LB media with 12 g/L agar (Difco, Erembodegem, Belgium) added.

The minimal medium for the shake flasks (MMfs) experiments contained 2.00 g/L (NH₄)2SO₄, 7.5 g/L KH₂PO₄, 17.5 g/L K₂HPO₄, 1.25 g/L Na-citrate, 0.25 g/L MgSO₄.7H₂O, 0.05 g/L tryptophan, from 10 up to 30 g/L glucose or another carbon source including but not limited to fructose, maltose, sucrose, glycerol and maltotriose when specified in the examples, 10 ml/L trace element mix and 10 ml/L Fe-citrate solution.

The medium was set to a pH of 7 with 1M KOH. Depending on the experiment lactose, LNB or LacNAc could be added as a precursor.

Complex medium, e.g. LB, was sterilized by autoclaving (121° C., 21′) and minimal medium by filtration (0.22 μm Sartorius). When necessary, the medium was made selective by adding an antibiotic (e.g. zeocin (20 mg/L)).

### Strains

Bacillus subtilis 168, available at Bacillus Genetic Stock Center (Ohio, USA).

### Plasmids for Gene Disruptions and Genomic Integrations

Plasmids for gene deletion via Cre/lox are constructed as described by Yan et al. (Appl & Environm. Microbial., September 2008, p 5556-5562). Gene disruption is done via homologous recombination with linear DNA and transformation via the electroporation as described by Xue et al. (J. microb. Meth. 34 (1999) 183-191). The method of gene knockouts is described by Liu et al. (Metab. Engine. 24 (2014) 61-69). This method uses 1000 bp homologies up- and downstream of the target gene.

Integrative vectors as described by Popp et al. (Sci. Rep., 2017, 7, 15158) are used as expression vector and could be further used for genomic integrations if necessary. A suitable promoter for expression can be derived from the part repository (iGem): sequence id: BBa_K143012, BBa_K823000, BBa_K823002 or BBa_K823003. Cloning can be performed using Gibson Assembly, Golden Gate assembly, Cliva assembly, LCR or restriction ligation.

### Heterologous and Homologous Expression

Genes that needed to be expressed, including the different exporters with SEQ ID NOs 01 to 21, 37 to 93 and 99 to 122, be it from a plasmid or from the genome, were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from an LB plate, in 150 μL LB and was incubated overnight at 37° C. on an orbital shaker at 800 rpm. This culture was used as inoculum for a 96-well square microtiter plate, with 400 μL MMsf medium by diluting 400×. Each strain was grown in multiple wells of the 96-well plate as biological replicates. These final 96-well culture plates were then incubated at 37° C. on an orbital shaker at 800 rpm for 72 h, or shorter, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations, as defined herein).

Also, a dilution of the cultures was made to measure the optical density at 600 nm. The cell performance index or CPI was determined by dividing the sialylated oligosaccharide concentrations, e.g. sialyllactose concentrations, measured in the whole broth by the biomass, in relative percentages compared to the reference strain. The biomass is empirically determined to be approximately ⅓^{rd} of the optical density measured at 600 nm. The sialylated oligosaccharide export ratio was determined by dividing the sialylated oligosaccharide concentrations measured in the supernatant by the sialylated oligosaccharide concentrations measured in the whole broth, in relative percentages compared to the reference strain.

Material and Methods Saccharomyces cerevisiae

### Media

Strains are grown on Synthetic Defined yeast medium with Complete Supplement Mixture (SD CSM) or on SD CSM drop-out medium containing 6.7 g/L Yeast Nitrogen Base without amino acids (YNB w/o AA, Difco), 20 g/L agar (Difco) (for solid cultures), 22 g/L glucose monohydrate or another carbon source including but not limited to fructose, maltose, sucrose, glycerol and maltotriose when specified in the examples and 0.79 g/L CSM or 0.77 g/L CSM drop-out mixture (MP Biomedicals). Depending on the experiment lactose, LNB or LacNAc could be added as a precursor.

### Strains

Saccharomyces cerevisiae BY4742 created by Brachmann et al. (Yeast (1998) 14:115-32) was used available in the Euroscarf culture collection. All mutant strains were created by homologous recombination or plasmid transformation using the method of Gietz (Yeast 11:355-360, 1995). Kluyveromyces marxianus lactis is available at the LMG culture collection (Ghent, Belgium).

### Plasmids and Gene Overexpression

Yeast expression plasmid p2a_2μ_exporter (Chan 2013 (Plasmid 70 (2013) 2-17)) was used for expression of foreign genes in Saccharomyces cerevisiae. This plasmid contains an ampicillin resistance gene and a bacterial origin of replication to allow for selection and maintenance in E. coli. The plasmid further contains the 2μ yeast ori and the URA3 selection marker for selection and maintenance in yeast. Finally, the plasmid can contain a beta-galactosidase expression cassette. All different exporters with SEQ ID NOs 01 to 21, 37 to 93 and 99 to 122 were cloned in the p2a_2μ_exporter plasmid. Cloning can be performed using Gibson Assembly, Golden Gate assembly, Cliva assembly, LCR or restriction ligation. All exporters are overexpressed using synthetic, constitutive promoters as described in Blazeck et al., 2012 (Biotechnology and Bioengineering, Vol. 109, No. 11) and Decoene et al., 2019 (PLoS ONE, 14(11)).

Plasmids were maintained in the host E. coli DH5alpha (F−, phi80dlacZdeltaM15, delta(lacZYA-argF)U169, deoR, recA1, endA1, hsdR17(rk−, mk+), phoA, supE44, lambda-, thi-1, gyrA96, relA1) bought from Invitrogen.

Gene Disruptions and Genomic Integrations For the construction of strains with gene knock-outs and for the introduction of genes in the yeast genome, knock-out and knock-in cassettes were PCR-amplified from template plasmids and transformed as linear DNA by the transformation technique of Gietz and Woods (2002). Template plasmids for knock-outs exist of a yeast auxotrophic marker (e.g. HISS, LEU2) flanked by 500 bp homologies of the target gene and are made in a pJET backbone. After integration, markers can be removed by the Cre/LoxP recombination system. Template plasmids for genomic knock-ins contain the different transcription units flanked by 500 bp homologies of the knock-in target site and are made in a pJET backbone. All genes are expressed using synthetic, constitutive promoters as described in Blazeck et al., 2012 (Biotechnology and Bioengineering, Vol. 109, No. 11) and Decoene et al., 2019 (PLoS ONE, 14(11)).

Plasmids were maintained in the host E. coli DH5alpha (F−, phi80dlacZdeltaM15, delta(lacZYA-argF)U169, deoR, recA1, endA1, hsdR17(rk−, mk+), phoA, supE44, lambda-, thi-1, gyrA96, relA1) bought from Invitrogen.

### Heterologous and Homologous Expression

Genes that needed to be expressed, be it from a plasmid or from the genome, were synthetically synthetized with one of the following companies: DNA2.0, Gen9, Twist Biosciences or IDT.

Expression could be further facilitated by optimizing the codon usage to the codon usage of the expression host. Genes were optimized using the tools of the supplier.

### Cultivation Conditions

A preculture of 96-well microtiter plate experiments was started from a cryovial or a single colony from a (selective) SD CSM plate, in 150 μL (selective SD CSM) and was incubated for 24 h at 30° C. on an orbital shaker at 800 rpm. This culture was used as inoculum for a 96-well square microtiter plate, with 400 μL MMsf medium by diluting 150×. Each strain was grown in multiple wells of the 96-well plate as biological replicates. These final 96-well culture plates were then incubated at 30° C. on an orbital shaker at 800 rpm for 72 h, or longer. At the end of the cultivation experiment samples were taken from each well to measure the supernatant concentration (extracellular sugar concentrations, after 5 min. spinning down the cells), or by boiling the culture broth for 15 min at 60° C. before spinning down the cells (=whole broth concentration, intra- and extracellular sugar concentrations).

Also, a dilution of the cultures was made to measure the optical density at 600 nm. The cell performance index or CPI was determined by dividing the sialylated oligosaccharide concentrations, e.g. sialyllactose concentrations, measured in the whole broth by the biomass, in relative percentages compared to the reference strain. The biomass is empirically determined to be approximately ⅓^{rd} of the optical density measured at 600 nm. The sialylated oligosaccharide export ratio was determined by dividing the sialylated oligosaccharide concentrations measured in the supernatant by the sialylated oligosaccharide concentrations measured in the whole broth, in relative percentages compared to the reference strain.

### Analytical Methods

### Optical Density

Cell density of the cultures was frequently monitored by measuring optical density at 600 nm (Implen Nanophotometer NP80, Westburg, Belgium or with a Spark 10M microplate reader, Tecan, Switzerland).

### Productivity

The specific productivity Qp is the specific production rate of the sialylated oligosaccharide product, typically expressed in mass units of product per mass unit of biomass per time unit (=g sialylated oligosaccharide /g biomass /h). The Qp value has been determined for each phase of the fermentation runs, i.e. Batch and Fed-Batch phase, by measuring both the amount of product and biomass formed at the end of each phase and the time frame each phase lasted.

The specific productivity Qs is the specific consumption rate of the substrate, e.g. sucrose, typically expressed in mass units of substrate per mass unit of biomass per time unit (=g sucrose /g biomass /h). The Qs value has been determined for each phase of the fermentation runs, i.e. Batch and Fed-Batch phase, by measuring both the total amount of sucrose consumed and biomass formed at the end of each phase and the time frame each phase lasted.

The yield on sucrose Ys is the fraction of product that is made from substrate and is typically expressed in mass unit of product per mass unit of substrate (=g sialylated oligosaccharide /g sucrose). The Ys has been determined for each phase of the fermentation runs, i.e. Batch and Fed-Batch phase, by measuring both the total amount of sialylated oligosaccharide produced and total amount of sucrose consumed at the end of each phase.

The yield on biomass Yx is the fraction of biomass that is made from substrate and is typically expressed in mass unit of biomass per mass unit of substrate (=g biomass /g sucrose). The Yp has been determined for each phase of the fermentation runs, i.e. Batch and Fed-Batch phase, by measuring both the total amount of biomass produced and total amount of sucrose consumed at the end of each phase.

The rate is the speed by which the product is made in a fermentation run, typically expressed in concentration of product made per time unit (=g sialylated oligosaccharide /L/ h). The rate is determined by measuring the concentration of sialylated oligosaccharide that has been made at the end of the Fed-Batch phase and dividing this concentration by the total fermentation time.

The lactose conversion rate is the speed by which lactose is consumed in a fermentation run, typically expressed in mass units of lactose per time unit (=g lactose consumed /h). The lactose conversion rate is determined by measurement of the total lactose that is consumed during a fermentation run, divided by the total fermentation time. Similar conversion rates can be calculated for other precursors such as Lacto-N-biose, N-acetyllactosamine, Lacto-N-tetraose, or Lacto-N-neotetraose.

### Growth Rate/Speed Measurement

The maximal growth rate (μMax) was calculated based on the observed optical densities at 600 nm using the R package grofit.

### Liquid Chromatography

Standards for 6′-sialyllactose, 3′-sialyllactose, LNT and LNnT were synthetized in house. Other standards such as but not limited to lactose, sucrose, glucose, glycerol, fructose were purchased from Sigma, and LST, LacNAc and LNB were purchased from Carbosynth. Carbohydrates were analyzed via an UPLC-RI (Waters, USA) method, whereby RI (Refractive Index) detects the change in the refraction index of a mobile phase when containing a sample. The sugars were separated in an isocratic flow using an Acquity BEH Amide column (Waters, USA) and a mobile phase containing 70% acetonitrile, 26% ammonium acetate buffer and 4% methanol. The column size was 2.1×100 mm with 1.7 μm particle size. The temperature of the column was set at 25° C. and the pump flow rate was 0.13 mL/min.

### Normalization of the Data

For all types of cultivation conditions, data obtained from the mutant strains was normalized against data obtained in identical cultivation conditions with reference strains having an identical genetic background as the mutant strains but lacking the membrane protein expression cassettes. The dashed horizontal line on each plot that is shown in the examples indicates the setpoint to which all adaptations were normalized. All data is given in relative percentages to that setpoint.

### Example 2: Membrane Proteins Identified that Enhance 6′-Sialyllactose (6′-SL) Production in an E. coli Host Cultivated 72 h in a Growth Experiment in Minimal Media Supplemented with 20 g/L Lactose

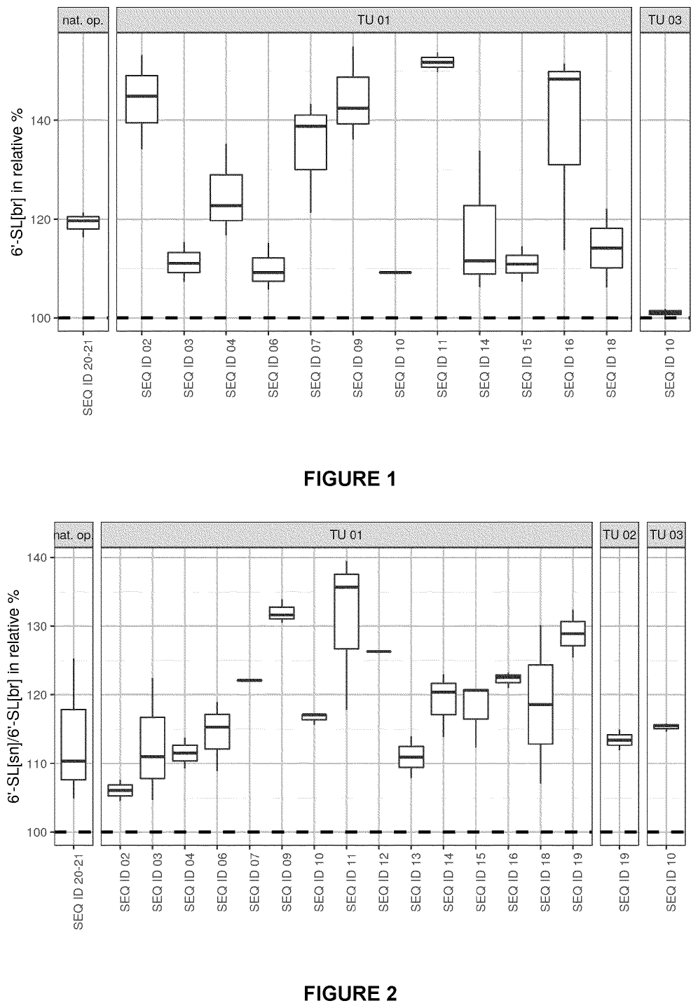

An experiment was set up to evaluate membrane proteins for their ability to enhance 6′-sialyllactose production of a host cell growing in minimal media supplemented with 20 g/L lactose. The membrane proteins with SEQ ID NOs 02, 03, 04, 06, 07, 09, 10, 11, 14, 15, 16, or 18 in TU 01, SEQ ID NO 10 in TU 03 or SEQ ID NOs 20 and 21 in their native transcriptional operon structure showed that they are able to enhance 6′-SL production that is being produced in a 6′-SL production host expressing a sialyllactose pathway with α-2,6-sialyltransferase ST1. Candidate genes were presented to the 6′-SL production hosts on a pSC101 plasmid. A growth experiment was performed according to the cultivation conditions provided in Example 1. FIG. 1 presents whole broth measurements of 6′-SL for the different strains in relative percentages compared to the respective reference strain.

### Example 3: Membrane Proteins Identified that Enhance 6′-SL Secretion in an E. coli Host Cultivated 72 h in a Growth Experiment in Minimal Media Supplemented with 20 g/L Lactose

An experiment was set up to evaluate membrane proteins for their ability to enhance 6′-sialyllactose secretion of a host cell growing in minimal media supplemented with 20 g/L lactose. The membrane proteins with SEQ ID NOs 02, 03, 04, 06, 07, 09, 10, 11, 12, 13, 14, 15, 16, 18 or 19 in TU 01, SEQ ID NO 19 in TU 02, SEQ ID NO 10 in TU 03 or SEQ ID 20 and 21 in their native transcriptional operon structure showed that they are able to enhance secretion of 6′-SL that is being produced intracellularly in a 6′-SL bacterial production host expressing a sialyllactose pathway with α-2,6-sialyltransferase ST1. Candidate genes were presented to the 6′-SL production hosts on a pSC101 plasmid. A growth experiment was performed according to the cultivation conditions provided in Example 1. FIG. 2 demonstrates the export ratio of 6′-SL in the strains, in relative percentages compared to the respective reference strain.

### Example 4: Membrane Proteins Identified that Enhance Growth Speed in an E. coli Host Cultivated 72 h in a Growth Experiment in Minimal Media Supplemented with 20 g/L Lactose

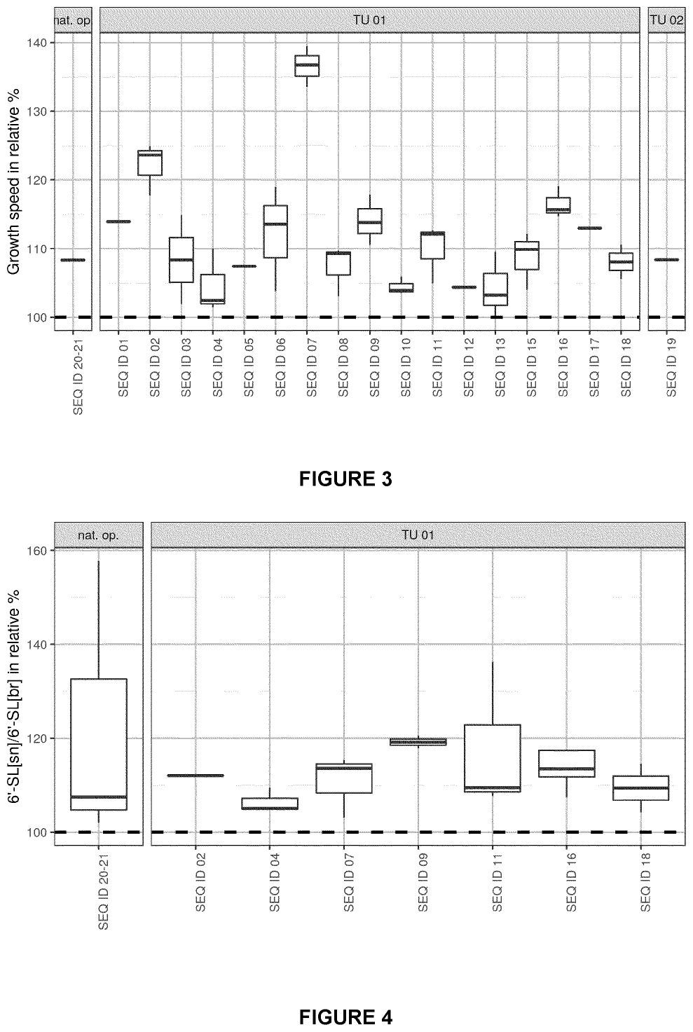

An experiment was set up to evaluate membrane proteins for their ability to influence growth speed of a host cell growing in minimal media supplemented with 20 g/L lactose. Membrane proteins with SEQ ID NOs 01, 02, 03, 04, 05, 06, 07, 08, 09, 10, 11, 12, 13, 15, 16, 17, or 18 in TU 01, SEQ ID NO 19 in TU 02 or SEQ ID NOs 20 and 21 in their native transcriptional operon structure showed to be able to enhance the growth speed of a 6′-SL production host expressing a sialyllactose pathway with α-2,6-sialyl transferase ST1 (SEQ ID NO 32). Candidate genes were presented to the 6′-SL production hosts on a pSC101 plasmid. A growth experiment was performed according to the cultivation conditions provided in Example 1. FIG. 3 demonstrates the growth speed of the strains, in relative percentages compared to the respective reference strain.

### Example 5: Membrane Proteins Identified that, when Integrated in the Host's Genome, Increase 6′-SL Secretion in an E. coli Host Cultivated 72 h in a Growth Experiment in Minimal Media Supplemented with 20 g/L Lactose

Another series of experiments was set up to evaluate the ability of membrane proteins integrated in the genome to increase 6′-sialyllactose secretion by a host cell cultivated for 72 h in minimal media supplemented with 20 g/L lactose. The membrane proteins with SEQ ID NOs 02, 04, 07, 09, 11, 16 or 18 in TU 01 or SEQ ID NOs 20 and 21 in their native transcriptional operon structure showed that they are able to enhance secretion of 6′-SL that is being produced intracellularly in a 6′-SL production host expressing a sialyllactose pathway with α-2,6-sialyltransferase ST1 (SEQ ID NO 32). The genes were presented to the genome of the 6′-SL production hosts as genomic knock-in. A growth experiment was performed according to the cultivation conditions provided in Example 1. FIG. 4 shows the 6′-SL export in relative percentages compared to the respective reference strain.

### Example 6: The Membrane Protein EcEntS (SEQ ID NO 09), when Varied in Gene Expression Levels and Integrated in the Host's Genome, can Further Enhance the Production and/or Secretion of 6′-SL in an E. coli Host Cultivated 72 h in a Growth Experiment in Minimal Media Supplemented with 20 g/L Lactose

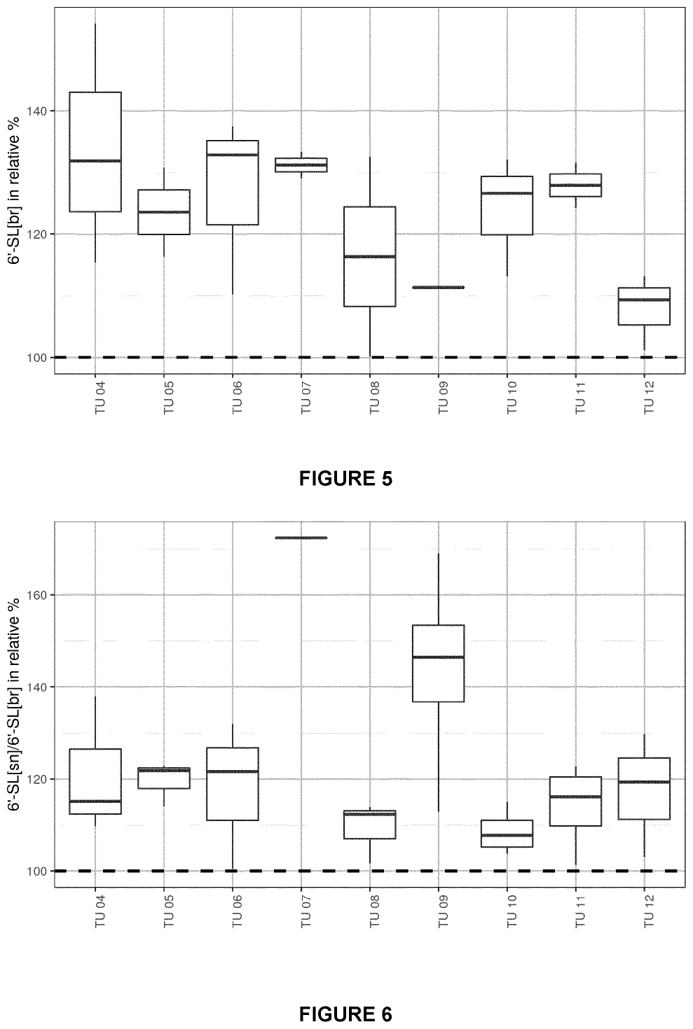

Another experiment was set up to evaluate the ability of the membrane protein with SEQ ID NO 09 when varied in gene expression and integrated in the genome, to enhance 6′-sialyllactose production and/or secretion of a host cell cultivated for 72 h in minimal media supplemented with 20 g/L lactose. The membrane protein with SEQ ID NO 09 was combined in transcription units TU 04, TU 05, TU 06, TU 07, TU 08, TU 09, TU 10, TU 11 or TU 12 and presented to the genome of the 6′-SL production hosts as genomic knock-in. These different transcriptional units with SEQ ID NO 09 showed that they were able to enhance 6′-SL production and/or secretion of 6′-SL that is being produced intracellularly in a 6′-SL production host expressing a sialyllactose pathway with α-2,6-sialyltransferase ST1 (SEQ ID NO 32). A growth experiment was performed according to the cultivation conditions provided in Example 1. FIG. 5 demonstrates whole broth measurements of 6′-SL whereas FIG. 6 shows the 6′-SL export, both times in relative percentages compared to the respective reference strains.

### Example 7: The Membrane Protein EcEntS (SEQ ID NO 09), when Expressed on Plasmid, Enhances the Export Ratio of 6′-SL in an E. coli Host in 5 L Fermentation Runs

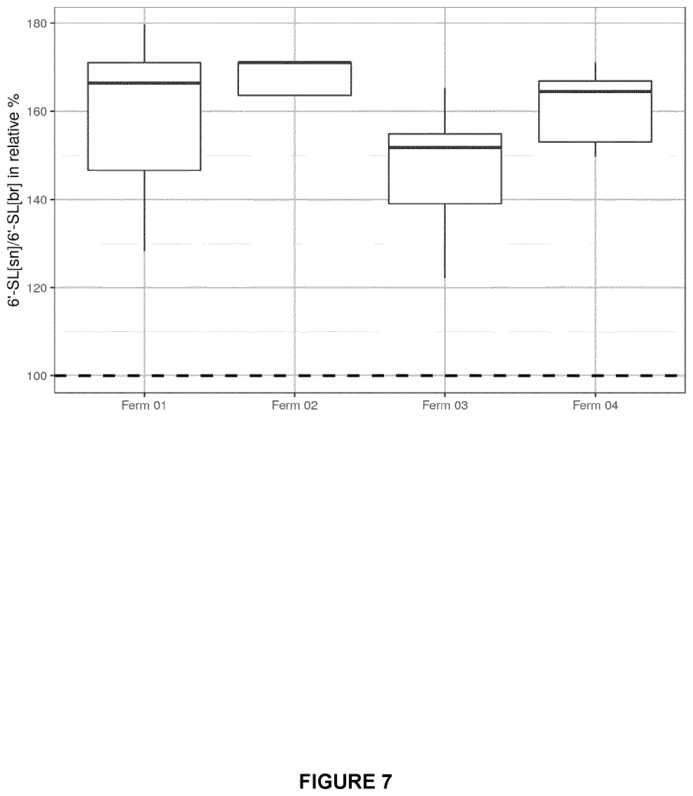

A 6′-SL producing E. coli host having the membrane protein gene with SEQ ID NO 09 expressed in TU 01 on a pSC101 plasmid and expressing the α-2,6-sialyltransferase ST1 (SEQ ID NO 32) from genome, or expressing the α-2,6-sialyltransferase ST1 (SEQ ID NO 32) from genome and plasmid, was evaluated for its productivity in bioreactor settings. For Ferm 03, an additional CMP-sialic acid synthetase and α-2,6-sialyltransferase ST1 were expressed from a p15A plasmid. Four fermentation runs were performed according to the conditions provided in Example 1. Also, a reference strain identical to the 6′-SL production host but lacking the membrane protein gene was analyzed in identical fermentation settings. FIG. 7 demonstrates the enhanced secretion of 6′-SL of the strain over-expressing the membrane protein EcEntS with SEQ ID NO 09 in the four different fermentation runs, relatively compared to the reference strain.

### Example 8: Additional Expression of a Membrane Protein Enhances the Production and/or Secretion of 3′-SL in an E. coli Host Cultivated 72 h in a Growth Experiment in Minimal Media Supplemented with 20 g/L Lactose

A 3′-SL producing E. coli as described in Example 1 wherein membrane proteins with SEQ ID NOs 01 up to 21 are expressed from plasmid or from the genome was cultivated for 72 h in minimal media supplemented with 20 g/L lactose. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of 3′-SL that is being produced in a 3′-SL production host expressing a sialyllactose pathway with α-2,3-sialyl transferase ST2 (SEQ ID NO 33).

### Example 9: Additional Expression of a Membrane Protein Enhances the Production and/or Secretion of Sialylated LNB (sLNB) in an E. coli Host Cultivated 72 h in a Growth Experiment in Minimal Media Supplemented with 20 g/L LNB

An sLNB producing E. coli as described in Example 1 wherein membrane proteins with SEQ ID NOs 01 up to 21 are expressed from plasmid or from the genome was cultivated for 72 h in minimal media supplemented with 20 g/L LNB. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of sLNB that is being produced in an sLNB production host expressing a sialyllactose pathway with an α-2,6-sialyl transferase ST1 in the case of 6′-sLNB or an α-2,3-sialyl transferase ST2 (SEQ ID NO 33) in the case of 3′-sLNB.

### Example 10: Additional Expression of a Membrane Protein Enhances the Production and/or Secretion of Sialylated LacNAc (sLacNAc) in an E. coli Host Cultivated 72 h in a Growth Experiment in Minimal Media Supplemented with 20 g/L LacNAc

An sLacNAc producing E. coli as described in Example 1 wherein membrane proteins with SEQ ID NOs 01 up to 21 are expressed from plasmid or from the genome was cultivated for 72 h in minimal media supplemented with 20 g/L LacNAc. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of sLacNAc that is being produced in an sLacNAc production host expressing a sialyllactose pathway with an α-2,6-sialyl transferase ST1 in the case of 6′-sLacNAc or an α-2,3-sialyl transferase ST2 (SEQ ID NO 33) in the case of 3′-sLacNAc.

### Example 11: Additional Expression of a Membrane Protein Enhances the Production and/or Secretion of LSTa in an E. coli Host Cultivated 72 h in a Growth Experiment in Minimal Media

An LSTa producing E. coli as described in Example 1 wherein membrane proteins with SEQ ID NOs 01 up to 21 are expressed from plasmid or from the genome was cultivated for 72 h in minimal media supplemented with 20 g/L lactose. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of LSTa that is being produced in an LSTa production host expressing an LNT pathway and a sialic acid pathway with an α-2,3-sialyl transferase ST2 (SEQ ID NO 33).

### Example 12: Additional Expression of a Membrane Protein Enhances the Production and/or Secretion of LSTb in an E. coli Host Cultivated 72 h in a Growth Experiment in Minimal Media

An LSTb producing E. coli as described in Example 1 wherein membrane proteins with SEQ ID NOs 01 up to 21 are expressed from plasmid or from the genome was cultivated for 72 h in minimal media supplemented with 20 g/L lactose. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of LSTb that is being produced in an LSTb production host expressing an LNT pathway and a sialic acid pathway with an α-2,6-sialyl transferase like ST6Gall or ST6GalII.

### Example 13: Additional Expression of a Membrane Protein Enhances the Production and/or Secretion of LSTc in an E. coli Host Cultivated 72 h in a Growth Experiment in Minimal Media

An LSTc producing E. coli as described in Example 1 wherein membrane proteins with SEQ ID NOs 01 up to 21 are expressed from plasmid or from the genome was cultivated for 72 h in minimal media supplemented with 20 g/L lactose. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of LSTc that is being produced in an LSTc production host expressing an LNnT pathway and a sialic acid pathway with an α-2,6-sialyl transferase ST1 (SEQ ID NO 32).

### Example 14: Additional Expression of a Membrane Protein Enhances the Production and/or Secretion of LSTd in an E. coli Host Cultivated 72 h in a Growth Experiment in Minimal Media

An LSTd producing E. coli as described in Example 1 wherein membrane proteins with SEQ ID NOs 01 up to 21 are expressed from plasmid or from the genome was cultivated for 72 h in minimal media supplemented with 20 g/L lactose. Candidate genes were combined in transcriptional unit TU 01, TU 02, TU 03 or their native transcriptional operon structure for SEQ ID NOs 20 and 21. A growth experiment was performed according to the cultivation conditions provided in Example 1. Said membrane proteins showed that they are able to enhance the production and/or secretion of LSTd that is being produced in an LSTd production host expressing an LNnT pathway and a sialic acid pathway with an α-2,3-sialyl transferase ST2 (SEQ ID NO 33).

### Example 15: Additional Expression of a Membrane Protein Enhances the Production and/or Secretion of 6′-SL or 3′-SL in a Bacillus subtilis Host

In another embodiment, these membrane proteins can be used to increase the production and/or secretion of 6′-SL or 3′-SL in another bacterial host like Bacillus subtilis. As described in WO1822225, a sialic acid producing B. subtilis strain is obtained by overexpressing the native fructose-6-P-aminotransferase (BsgImS) to enhance the intracellular glucosamine-6-phosphate pool. Further on, the enzymatic activities of the genes nagA, nagB and gamA were disrupted by genetic knockouts and a glucosamine-6-P-aminotransferase from S. cerevisiae (ScGNA1), an N-acetylglucosamine-2-epimerase from Bacteroides ovatus (BoAGE) and a sialic acid synthase from Campylobacter jejuni (CjneuB) were overexpressed on the genome. In addition, a lactose permease from E. coli (EclacY) was integrated in the genome to establish lactose uptake.

To allow production of 6′-SL, a CMP-sialic acid synthetase from Neisseria meningitidis (NmneuA, SEQ ID NO 31) and a sialyltransferase from Photobacterium damselae (PdbST, SEQ ID NO 32) were overexpressed.

To allow production of 3′-SL, a CMP-sialic acid synthetase from Neisseria meningitidis (NmneuA, SEQ ID NO 31) and a sialyltransferase from Neisseria meningitidis (NmST, SEQ ID NO 33) were overexpressed.

In these 6′-SL or 3′-SL producing B. subtilis host strains, membrane proteins with SEQ ID NOs 01 up to 21 are expressed from plasmid or from the genome and cultivated for 72 h in minimal media supplemented with 20 g/L lactose. Candidate genes were combined in transcriptional unit TU 01, TU 02, TU 03 or their native transcriptional operon structure for SEQ ID 20 and 21. A growth experiment was performed according to the cultivation conditions for B. subtilis as provided in Example 1. Said membrane proteins showed that they are able to enhance the production and/or secretion of 6′-SL that is being produced in a 6′-SL production B. subtilis host expressing a sialyllactose pathway with α-2,6-sialyl transferase ST1 or 3′-SL that is being produced in a 3′-SL production B. subtilis host expressing a sialyllactose pathway with α-2,3-sialyl transferase ST2 (SEQ ID NO 33).

In another embodiment, these membrane proteins could be used to increase the production and/or secretion of other sialylated oligosaccharides like but not limited to sLNB, sLacNAc, LSTa, LSTb, LSTc and LSTd in a Bacillus subtilis host strain.

### Example 16: Additional Expression of a Membrane Protein Enhances the Production and/or Secretion of 6′-SL or 3′-SL in Saccharomyces cerevisiae

In another embodiment, these membrane proteins can be used to increase the production and/or secretion of 6′-SL or 3′-SL in a eukaryotic organism like Saccharomyces cerevisiae. A strain with increased flux towards N-acetylglucosamine-6-phosphate was made by overexpressing a fructose-6-P-aminotransferase mutant from E. coli (EcgImS*54, as described by Deng et al. (Biochimie 88, 419-29 (2006)), an N-acetylglucosamine-2-epimerase from Bacteroides ovatus (BoAGE) and a sialic acid synthase from Campylobacter jejuni (CjneuB). Also, a lactose permease from Kluyveromyces lactis (KILAC12, SEQ ID NO 23) was expressed to establish lactose import.

To allow production of 6′-SL, a CMP-sialic acid synthetase from Neisseria meningitidis (NmneuA) and a sialyltransferase from Photobacterium damselae (PdbST, SEQ ID NO 32) were overexpressed. To allow production of 3′-SL, a CMP-sialic acid synthetase from Neisseria meningitidis (NmneuA, SEQ ID NO 31) and a sialyltransferase from Neisseria meningitidis (NmST, SEQ ID NO 33) were overexpressed. The different gene modules were integrated in the yeast genome by homologous recombination; EcgImS*54 and BoAGE were introduced at the LEU2 locus, KILAC12 and CjneuB were introduced at the HIS3 locus, and NmneuA and PdbST or NmneuA and NmST were introduced at the LYS2 locus. All genes are expressed by synthetic, constitutive yeast promoters (Blazeck et al., 2012 (Biotechnology and Bioengineering, Vol. 109, No. 11) and Decoene et al., 2019 (PLoS ONE, 14(11))) as described in Example 1 and are introduced by the transformation technique of Gietz and Woods (2002).

In this 6′-SL or 3′-SL producing S. cerevisiae host strain, membrane proteins with SEQ ID NOs 01 up to 21 are expressed from a 2-micron plasmid containing a URA3 auxotrophic marker gene or from the genome and cultivated for 72 h in minimal media supplemented with 20 g/L lactose. Candidate genes were expressed using synthetic, constitutive yeast promoters (Blazeck et al., 2012 (Biotechnology and Bioengineering, Vol. 109, No. 11) and Decoene et al., 2019 (PLoS ONE, 14(11))). A growth experiment was performed according to the cultivation conditions for S. cerevisiae as provided in Example 1. Said membrane proteins showed that they are able to enhance the production and/or secretion of 6′-SL that is being produced in a 6′-SL production S. cerevisiae host expressing a sialyllactose pathway with α-2,6-sialyl transferase ST1, or 3′-SL that is being produced in a 3′-SL production S. cerevisiae host expressing a sialyllactose pathway with α-2,3-sialyl transferase ST2 (SEQ ID NO 33).

In another embodiment, these membrane proteins could be used to increase the production and/or secretion of other sialylated oligosaccharides like but not limited to sLNB, sLacNAc, LSTa, LSTb, LSTc and LSTd in a Saccharomyces cerevisiae host strain.

### Example 17: Membrane Proteins Identified that Obtain Ratios for Supernatant Concentration Over Whole Broth Concentration of 6′-SL Higher than 0.65 in an E. coli Host Cultivated 72 h in a Growth Experiment in Minimal Media Supplemented with 20 g/L Lactose

An experiment was set up to evaluate membrane proteins for their ability to excrete 6′-sialyllactose by a host cell growing in minimal media supplemented with 20 g/L lactose and having a supernatant concentration over whole broth concentration ratio higher than 0.65. The membrane proteins with SEQ ID NOs 02, 03, 04, 06, 07, 09, 10, 11, 12, 13, 14, 15, 16, 18 or 19 in TU 01, SEQ ID NO 19 in TU 02, SEQ ID NO 10 in TU 03 or SEQ ID 20 and 21 in their native transcriptional operon structure showed that they had ratios of supernatant concentration over whole broth concentration of 6′-SL higher than 0.65 produced by a 6′-SL bacterial production host expressing a sialyllactose pathway with α-2,6-sialyltransferase ST1 (SEQ ID NO 32). Candidate genes were presented to the 6′-SL production hosts on a pSC101 plasmid. A growth experiment was performed according to the cultivation conditions provided in Example 1. Table 3 demonstrates the mean and standard deviation of supernatant over whole broth concentration ratios of 6′-SL in these strains and in a reference strain lacking any extra overexpressed membrane protein.

TABLE 3

| SEQ ID NO | TU NO | mean ratio | stdev ratio |
|---|---|---|---|
| SEQ ID 02 | TU 01 | 0.69 | 0.014 |
| SEQ ID 03 | TU 01 | 0.73 | 0.058 |
| SEQ ID 04 | TU 01 | 0.72 | 0.021 |
| SEQ ID 06 | TU 01 | 0.74 | 0.033 |
| SEQ ID 07 | TU 01 | 0.79 | NA |
| SEQ ID 09 | TU 01 | 0.86 | 0.011 |
| SEQ ID 10 | TU 01 | 0.76 | 0.005 |
| SEQ ID 10 | TU 03 | 0.75 | 0.004 |
| SEQ ID 11 | TU 01 | 0.85 | 0.075 |
| SEQ ID 12 | TU 01 | 0.82 | NA |
| SEQ ID 13 | TU 01 | 0.72 | 0.028 |
| SEQ ID 14 | TU 01 | 0.77 | 0.031 |
| SEQ ID 15 | TU 01 | 0.76 | 0.032 |
| SEQ ID 16 | TU 01 | 0.79 | 0.007 |
| SEQ ID 18 | TU 01 | 0.77 | 0.106 |
| SEQ ID 19 | TU 02 | 0.74 | 0.014 |
| SEQ ID 19 | TU 01 | 0.84 | 0.032 |
| SEQ ID 20-21 | nat. op. | 0.74 | 0.061 |
| NA - reference | NA | 0.65 | 0.022 |

### Example 18: Ratios for Supernatant Concentration Over Whole Broth Concentration of 6′-SL, Produced by an E. coli Expressing Membrane Protein EcEntS (SEQ ID NO 09) on Plasmid and Grown in 5 L Fermentation Runs, are Increased Compared to a Reference Strain Lacking, the Overexpressed Membrane Protein Gene EcEntS and Cultivated in an Identical Fermentation Setting

A 6′-SL producing E. coli host having the membrane protein gene with SEQ ID NO 09 expressed in TU 01 on a pSC101 plasmid and expressing the α-2,6-sialyltransferase ST1 (SEQ ID NO 32) from genome, or expressing the α-2,6-sialyltransferase ST1 (SEQ ID NO 32) from genome and plasmid, was evaluated for the 6′-SL ratio of supernatant concentration over whole broth concentration during different time points in bioreactor settings. For Ferm 03, an additional CMP-sialic acid synthetase and α-2,6-sialyltransferase ST1 (SEQ ID NO 32) were expressed from a p15A plasmid. Four fermentation runs were performed according to the conditions provided in Example 1. Also, a reference strain identical to the 6′-SL production host but lacking the membrane protein gene was analyzed in identical fermentation settings. Table 4 demonstrates the enhanced ratio of supernatant over whole broth concentration of 6′-SL taken during different time points of 5 L fermentation runs from the strain overexpressing the membrane protein EcEntS with SEQ ID NO 09. From t1 onwards, all ratios are higher than the reference on that specific time point.

TABLE 4

| Timepoint | Reference | Ferm 01 | Ferm 02 | Ferm 03 | Ferm 04 |
|---|---|---|---|---|---|
| t1 | 0.50 | 0.67 | 0.60 | 0.71 | 0.88 |
| t2 | 0.67 | 0.75 | 1.00 | 0.90 | 0.92 |
| t3 | 0.55 | 0.86 | 0.75 | 0.80 | 0.88 |
| t4 | 0.47 | 0.77 | 1.00 | 0.80 | 0.89 |
| t5 | 0.52 | 0.97 | 1.00 | 0.88 | 1.00 |
| t6 | 0.55 | 1.00 | 0.98 | 0.90 | 0.97 |
| t7 | 0.61 | 0.98 | 0.96 | 0.85 | 0.98 |
| t8 | 0.56 | 0.96 | 1.00 | 0.91 | 0.98 |
| t9 | 0.71 | 1.05 | 1.00 | 0.92 | 0.96 |
| t10 | 0.72 | 1.05 | NA | 0.97 | NA |
| Mean ratio | 0.58 | 0.89 | 0.92 | 0.86 | 0.94 |
| Stdev ratio | 0.087 | 0.173 | 0.145 | 0.074 | 0.048 |

### Example 19: Example Identification of Siderophore Exporters in Neighborhood of Siderophore Biosynthesis Genes Using EFI-GNT

A first set of membrane proteins were found by identifying the EggNOG4.5.1 ortholog family members of the membrane proteins found in the neighborhood of siderophore biosynthesis genes. Protein identifiers belonging to dihydroxybenzoate-2, 3-dehydrogenase (cd05331), isochorismate pyruvate lyase (IPR019996), L-ornithine N5-monooxygenase (COG3486) and N(6)-hydroxylysine synthase (PF04183) were extracted from UniProtKB/trembl. These identifiers were used as input in https://efi.igb.illinois.edu/efi-gnt/. EFI-GNT allows exploration of the genome neighborhoods. A neighborhood window size of 10 was selected. Neighboring genes were classified based on Eggnog4.5.1 orthology using a stand-alone version of eggnog-mapperv1 (https://github.com/jhcepas/eggnog-mapper/releases). The most frequent observed putative siderophore transporter NOG and bactNOG orthology families are present near siderophore biosynthesis genes are shown in Table 5.

TABLE 5

| Superfamily | NOG | bactNOG |
|---|---|---|
| MFS | COG0477, 0ZVQG, 0ZPI7, 0ZVXV, 0XNN3, | 05E8G, 08HFG, 089VA, 07TNI, 05C0R, |
| | COG3182, 0ZW7F, 0XP7I, 0ZVCH, 0XQZX, | 07Y9F, 05CSH, 05QRD, 05EDF, 05C6X, |
| | 0XNQK, 0ZVYD, COG2271, 0XNNX, 0ZZWT, | 08NGX, 05C2C, 07FU4, 07U9Z, 080SS, |
| | COG2814, 0ZITE, 0ZVC8, 0XT98, 0XNQ6, | 07SFI, 05EYM, 05C57, 08E7F, 07QF7, |
| | 0YAQV, 0ZVQA, COG2211, COG3104 | 05CSP, 07UZE, 07VHC, 08EFJ, 05CT4, |
| | | 05FCD, 07YDJ, 08MMW, 08TKV |
| ABC | COG1132, COG1173, COG0842, COG4615, | 05BZ1, 05IBP, 05CK8, 05IUH, 05D6C, |
| | COG0577, COG2274, COG4618, COG4172, | 08E0J, 08JJ6, 08JJA, 05FDX, 05EGG, 08JN3, |
| | COG5265, COG1136, 0XPIZ, COG0444 | 08N1B, 05IDI, 08ITX |
| RND | COG2409, COG0841, COG3696, COG0845, | 05EYF, 07R13, 05BZS, 08IJF |
| | COG1033 | |
| MOP | COG0534, 0Y3TF, COG2244 | 05UQX, 05C3S, 07U3M, 07R73, 07T1S, |
| | | 07TJ5, 07XCD |

### Example 20: Example Identification of Siderophore Exporters in Siderophore Biosynthesis Gene Clusters by Antismash

A second set of membrane proteins were found by identifying the EggNOG4.5.1 ortholog family members of the membrane proteins found in siderophore biosynthesis gene clusters by antiSMASH (https://antismash.secondarymetabolites.org/#!/download). AntiSMASH allows the rapid genome-wide identification, annotation and analysis of secondary metabolite biosynthesis gene clusters in bacterial and fungal genomes. Complete representative fungal and bacterial genome assemblies from ncbi (https://www.ncbi.nlm.nih.gov/assembly) were used as input in the stand-alone antiSMASH5.0 version. Siderophore biosynthesis clusters were annotated using a stand-alone version of eggnog-mapperv1 (https://github.com/jhcepas/eggnog-mapper/releases). The most frequent observed putative siderophore transporter NOG and bactNOG orthology families are present near siderophore biosynthesis genes are shown in Table 6.

TABLE 6

| Superfamily | NOG | bactNOG |
|---|---|---|
| MFS | COG0477, 0ZW7F, 0ZVCH, 0ZPI7, 0XNN3, | 05QRD, 07Y9F, 05C6X, 089VA, 05C0R, |
| | 0XNNX, 1269U, 0XP7I, 0ZW8Z | 05C2C, 05CSH, 05E8G, 07SFI, 07VHC, |
| | | 07XMP, 08E7F |
| ABC | COG1132, COG0842, 0XPIZ, COG4779, | 05BZ1, 05EGG, 05TVJ, 05DHS, 05CM4, |
| | COG4606, COG0601, COG1108, COG3182, | 07RUJ |
| | COG4214, COG4605 | |
| RND | COG2409 | |
| MOP | COG0534, 0XPYW | 05C3S, 07XCD, 05DJC |

### Example 21: Membrane Proteins Identified that Enhance 6′-SL Production in an E. coli Host Cultivated 72 h in a Growth Experiment in Minimal Media Supplemented with 20 g/L Lactose

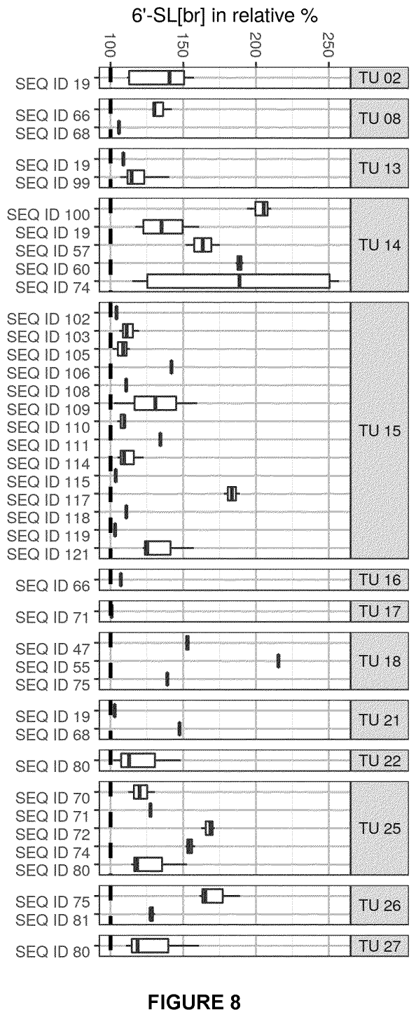

An experiment was set up to evaluate membrane proteins for their ability to enhance 6′-sialyllactose production of a host cell growing in minimal media supplemented with 20 g/L lactose. In addition, part of the membrane proteins was assembled in different transcription units to evaluate the effect of different expression levels on production. The membrane proteins with SEQ ID NO 19 in TU 02, SEQ ID NOs 66 and 68 in TU08, SEQ ID NOs 19 and 99 in TU 13, SEQ ID NOs 100, 19, 57, 60 and 74 in TU 14, SEQ ID NOs 102, 103, 105, 106, 108, 109, 110, 111, 114, 115, 117, 118, 119 and 121 in TU 15, SEQ ID NO 66 in TU 16, SEQ ID NO 71 in TU 17, SEQ ID NOs 47, 55 and 75 in TU 18, SEQ ID NOs 19 and 68 in TU 21, SEQ ID NO 80 in TU 22, SEQ ID NOs 70, 71, 72, 74 and 80 in TU 25, SEQ ID NOs 75 and 81 in TU 26 and SEQ ID NO 80 in TU 27 showed that they are able to enhance 6′-SL production that is being produced in a 6′-SL production host expressing a sialyllactose pathway with α-2,6-sialyltransferase ST1. Candidate genes were presented to the 6′-SL production hosts on a pSC101 plasmid. A growth experiment was performed according to the cultivation conditions provided in Example 1. FIG. 8 presents whole broth measurements of 6′-SL for the different strains in relative percentages compared to the respective reference strain.

### Example 22: Membrane Proteins Identified that Enhance 6′-SL Secretion in an E. coli Host Cultivated 72 h in a Growth Experiment in Minimal Media Supplemented with 20 g/L Lactose

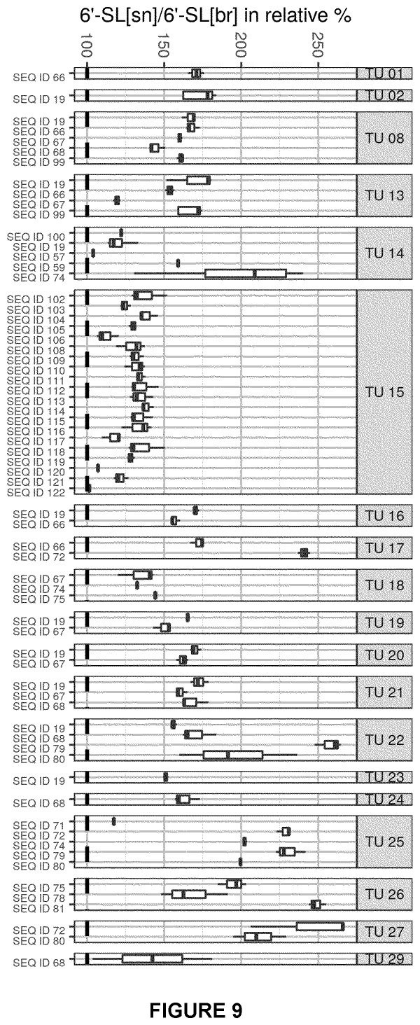

An experiment was set up to evaluate membrane proteins for their ability to enhance 6′-sialyllactose secretion of a host cell growing in minimal media supplemented with 20 g/L lactose. In addition, part of the membrane proteins was assembled in different transcription units to evaluate the effect of different expression levels on secretion. The membrane proteins with SEQ ID NO 66 in TU 01, SEQ ID NO 19 in TU 02, SEQ ID NOs 19, 66, 67, 68 and 99 in TU 08, SEQ ID NOs 19, 66, 67 and 99 in TU 13, SEQ ID NOs 100, 19, 57, 59 and 74 in TU 14, SEQ ID NOs 102, 103, 104, 105, 106, 108, 109, 110, 111, 112, 113, 114, 115, 116, 117, 118, 119, 120, 121 and 122 in TU 15, SEQ ID NOs 19 and 66 in TU 16, SEQ ID NOs 66 and 72 in TU 17, SEQ ID NOs 67, 74 and 75 in TU 18, SEQ ID NOs 19 and 67 in TU 19 and TU 20, SEQ ID NOs 19, 67 and 68 in TU 21, SEQ ID NOs 19, 68, 79 and 80 in TU 22, SEQ ID NO 19 in TU 23, SEQ ID NO 68 in TU 24, SEQ ID NOs 71, 72, 74, 79 and 80 in TU 25, SEQ ID NOs 75, 78 and 81 in TU 26, SEQ ID NOs 72 and 80 in TU 27 and SEQ ID NO 68 in TU 29 showed that they are able to enhance secretion of 6′-SL that is being produced intracellularly in a 6′-SL bacterial production host expressing a sialyllactose pathway with α-2,6-sialyltransferase ST1. Candidate genes were presented to the 6′-SL production hosts on a pSC101 plasmid. The TUs used are enlisted in Table 2. A growth experiment was performed according to the cultivation conditions provided in Example 1. FIG. 9 demonstrates the export ratio of 6′-SL in the strains, in relative percentages compared to the respective reference strain.

### Example 23: Membrane Proteins Identified that Enhance Growth Speed in an E. coli Host Cultivated 72 h in a Growth Experiment in Minimal Media Supplemented with 20 g/L Lactose

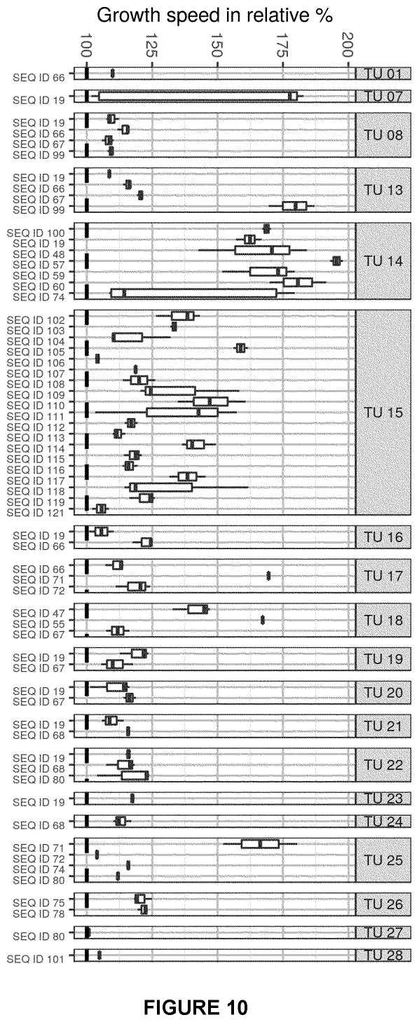

An experiment was set up to evaluate membrane proteins for their ability to influence growth speed of a host cell growing in minimal media supplemented with 20 g/L lactose. Membrane proteins with SEQ ID NO 66 in TU 01, SEQ ID NO 19 in TU 07, SEQ ID NOs 19, 66, 67 and 99 in TU 08 and TU 13, SEQ ID NOs 100, 19, 48, 57, 59, 60 and 74 in TU 14, SEQ ID NOs 102, 103, 104, 105, 106, 107, 108, 109, 110, 111, 112, 113, 114, 115, 116, 117, 118, 119 and 121 in TU 15, SEQ ID NOs 19 and 66 in TU 16, SEQ ID NOs 66, 71 and 72 in TU 17, SEQ ID NOs 47, 55 and 67 in TU 18, SEQ ID NOs 19 and 67 in TU 19 and TU 20, SEQ ID NOs 19 and 68 in TU 21, SEQ ID NOs 19, 68 and 80 in TU 22, SEQ ID NO 19 in TU 23, SEQ ID NO 68 in TU 24, SEQ ID NOs 71, 72, 74 and 80 in TU 25, SEQ ID NOs 75 and 78 in TU 26, SEQ ID NO 80 in TU 27 and SEQ ID NO 101 in TU 28 showed to be able to enhance the growth speed of a 6′-SL production host expressing a sialyllactose pathway with α-2,6-sialyl transferase ST1 (SEQ ID NO 32). Candidate genes were presented to the 6′-SL production hosts on a pSC101 plasmid. The TUs used are enlisted in Table 2. A growth experiment was performed according to the cultivation conditions provided in Example 1. FIG. 10 demonstrates the growth speed of the strains, in relative percentages compared to the respective reference strain.

### Example 24: Additional Expression of a Membrane Protein Enhances the Production and/or Secretion of 3′-SL in an E. coli Host Cultivated 72 h in a Growth Experiment in Minimal Media Supplemented with 20 g/L Lactose

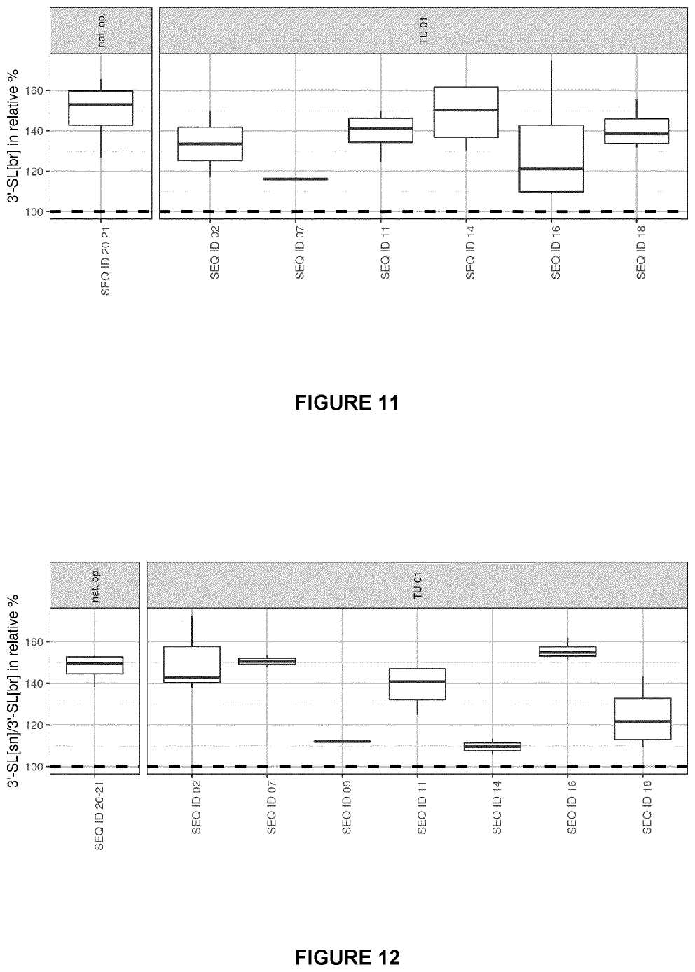

An experiment was set up to evaluate membrane proteins for their ability to enhance production and/or secretion of 3′-sialyllactose in a host cell growing in minimal media supplemented with 20 g/L lactose. Membrane proteins with SEQ ID NOs 02, 07, 11, 14, 16 and 18 in TU 01 and SEQ ID NOs 20 and 21 in their natural operon structure showed that they are able to enhance 3′-SL production that is being produced in a 3′-SL production host expressing a sialyllactose pathway with α-2,3-sialyl transferase ST2 (SEQ ID NO 33). Membrane proteins with SEQ ID NOs 02, 07, 09, 11, 14, 16 and 18 in TU 01 and SEQ ID NOs 20 and 21 in their natural operon structure showed that they are able to enhance secretion of 3′-SL that is being produced intracellularly in a 3′-SL production host expressing a sialyllactose pathway with α-2,3-sialyl transferase ST2 (SEQ ID NO 33). Candidate genes were presented to the 3′-SL production hosts on a pSC101 plasmid. A growth experiment was performed according to the cultivation conditions provided in Example 1. FIG. 11 presents whole broth measurements of 3′-SL for the different strains in relative percentages compared to the respective reference strain. FIG. 12 demonstrates the export ratio of 3′-SL in the strains, in relative percentages compared to the respective reference strain.

### Example 25: The Membrane Protein EcEntS (SEQ ID NO 09), when Expressed on Plasmid, Leads to Higher 6′-SL Titers in an E. coli Host in 5 L Fermentation Runs

A 6′-SL producing E. coli host having the membrane protein gene with SEQ ID NO 09 expressed in TU 01 on a pSC101 plasmid and expressing the α-2,6-sialyl transferase ST1 (SEQ ID NO 32) from genome, or expressing the α-2,6-sialyl transferase ST1 (SEQ ID NO 32) from genome and plasmid, was evaluated for its productivity in bioreactor settings (5 L fermenter). Four fermentation runs were performed according to the conditions provided in Example 1. Also, a reference strain identical to the 6′-SL production host but lacking the membrane protein gene was analyzed in identical fermentation settings. At the end of all fermentation runs, the 6′-SL titers measured in supernatant and whole broth samples varied between 50 g/L and 65 g/L for the strains expressing the membrane protein EcEntS from E. coli (SEQ ID NO 09). The reference strain had 6′-SL titers between 20 g/L and 40 g/L measured in supernatant and whole broth samples, which shows the positive effect of the membrane protein EcEntS (SEQ ID NO 09) on 6′-SL production in 5 L fermentation runs.

1-34. (canceled)

35. Method for the production of sialylated oligosaccharide by a genetically modified cell, comprising the steps of:
providing a cell capable of producing sialylated oligosaccharide, said cell comprising at least one nucleic acid sequence coding for an enzyme for sialylated oligosaccharide synthesis,
said cell genetically modified for i) overexpression of an endogenous membrane protein, ii) expression or overexpression of a homologous membrane protein, and/or iii) expression or overexpression of a heterologous membrane protein culturing the cell in a medium under conditions permissive for the production of sialylated oligosaccharide.

36. Method according to claim 35, wherein said cell is genetically modified for the production of sialylated oligosaccharide and wherein said genetically modified cell a) excretes sialylated oligosaccharide at a ratio of the supernatant concentration to whole broth concentration higher than 0.5 and/or b) has an enhanced production of sialylated oligosaccharide compared to a cell with the same genetic makeup but lacking the i) overexpression of the endogenous membrane protein, ii) expression or overexpression of the homologous membrane protein and/or iii) expression or overexpression of the heterologous membrane protein, respectively.

37. Method according to claim 35, wherein said membrane protein comprises
i) an amino acid sequence encoding a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08116, 08JJA, 05FDX, 05EGG, 08JN3, 08N1B, 05IDI, 08ITX, 05TVJ, 05DHS, 05CM4, 07RUJ, 05EYF, 07R13, 05BZ5, 08IJF, 05UQX, 05C3S, 07U3M, 07R73, 07T1S, 07TJ5, 07XCD, 05DJC, 07RBJ, 05CXP; or
ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
iv) an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid.

38. Method according to claim 35, wherein
i) when said membrane protein is a siderophore exporter, said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides;
ii) when said membrane protein is an ABC transporter, said membrane protein is selected from oppF from Escherichia coli K12 MG1655 with SEQ ID NO 18, lmrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides;
iii) when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides;
iv) when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.

39. Method according to claim 35, the method further comprising at least one of the following steps:
i) Adding to the culture medium a precursor feed comprising at least 50, more preferably at least 75, more preferably at least 100, more preferably at least 120, more preferably at least 150 gram of precursor per litre of initial reactor volume wherein the total reactor volume ranges from 250 mL (millilitre) to 10.000 m3 (cubic meter), preferably in a continuous manner, and preferably so that the final volume of the culture medium is not more than three-fold, preferably not more than two-fold, more preferably less than 2-fold of the volume of the culture medium before the addition of said precursor feed;
ii) Adding a precursor feed in a continuous manner to the culture medium over the course of 1 day, 2 days, 3 days, 4 days, 5 days by means of a feeding solution;
iii) Adding a precursor feed in a continuous manner to the culture medium over the course of 1 day, 2 days, 3 days, 4 days, 5 days by means of a feeding solution and wherein the concentration of said precursor feeding solution is 50 g/L, preferably 75 g/L, more preferably 100 g/L, more preferably 125 g/L, more preferably 150 g/L, more preferably 175 g/L, more preferably 200 g/L, more preferably 225 g/L, more preferably 250 g/L, more preferably 275 g/L, more preferably 300 g/L, more preferably 325 g/L, more preferably 350 g/L, more preferably 375 g/L, more preferably, 400 g/L, more preferably 450 g/L, more preferably 500 g/L, even more preferably, 550 g/L, most preferably 600 g/L; and wherein preferably the pH of said solution is set between 3 and 7 and wherein preferably the temperature of said feed solution is kept between 20° C. and 80° C.;
iv) Said method resulting in sialylated oligosaccharide concentration of at least 50 g/L, preferably at least 75 g/L, more preferably at least 90 g/L, more preferably at least 100 g/L, more preferably at least 125 g/L, more preferably at least 150 g/L, more preferably at least 175 g/L, more preferably at least 200 g/L in the final volume of said culture medium.

40. Method according to claim 39, wherein the precursor feed is accomplished by adding precursor from the beginning of the cultivating in a concentration of at least 5 mM, preferably in a concentration of 30, 40, 50, 60, 70, 80, 90, 100, 150 mM, more preferably in a concentration >300 mM.

41. Method according to claim 39, wherein said precursor feed is accomplished by adding precursor to the cultivation medium in a concentration, such, that throughout the production phase of the cultivation a precursor concentration of at least 5 mM, preferably 10 mM or 30 mM is obtained.

42. Method according to claim 39, wherein the host cells are cultivated for at least about 60, 80, 100, or about 120 hours or in a continuous manner.

43. Method according to claim 35, wherein a precursor feed is added to the culture medium and wherein precursor is chosen from the group comprising lactose, lacto-N-biose (LNB), lacto-N-triose, lacto-N-tetraose (LNT), lacto-N-neotetraose (LNnT), N-acetyl-lactosamine (LacNAc), lacto-N-pentaose (LNP), lacto-N-neopentaose, para lacto-N-pentaose, para lacto-N-neopentaose, lacto-N-novopentaose I, lacto-N-hexaose (LNH), lacto-N-neohexaose (LNnH), para lacto-N-neohexaose (pLNnH), para lacto-N-hexaose (pLNH), lacto-N-heptaose, lacto-N-neoheptaose, para lacto-N-neoheptaose, para lacto-N-heptaose, lacto-N-octaose (LNO), lacto-N-neooctaose, iso lacto-N-octaose, para lacto-N-octaose, iso lacto-N-neooctaose, novo lacto-N-neooctaose, para lacto-N-neooctaose, iso lacto-N-nonaose, novo lacto-N-nonaose, lacto-N-nonaose, lacto-N-decaose, iso lacto-N-decaose, novo lacto-N-decaose, lacto-N-neodecaose, galactosyllactose, a lactose extended with 1, 2, 3, 4, 5, or a multiple of N-acetyllactosamine units and/or 1, 2, 3, 4, 5, or a multiple of lacto-N-biose units, and oligosaccharide containing 1 or multiple N-acetyllactosamine units and/or 1 or multiple lacto-N-biose units or an intermediate into sialylated oligosaccharide, fucosylated and sialylated versions thereof.

44. Method according to claim 35, wherein a carbon and energy source, preferably sucrose, glucose, fructose, glycerol, maltose, maltodextrines, trehalose, polyols, starch, succinate, malate, pyruvate, lactate, ethanol, citrate, lactose, is also added, preferably continuously to the culture medium, preferably with the precursor.

45. Method according to claim 35, wherein a first phase of exponential cell growth is provided by adding a carbon-based substrate, preferably glucose or sucrose, to the culture medium before the lactose is added to the culture medium in a second phase.

46. Method according to claim 35, wherein said sialylated oligosaccharide is 6′-sialyllactose, 3′-sialyllactose, 3-fucosyl-3′-sialyllactose (3′-O-sialyl-3-0-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.

47. Method according to claim 35, wherein the method is producing a mixture of sialylated oligosaccharides.

48. Method according to claim 35, wherein said genetically modified cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal, preferably the cell is an Escherichia coli cell.

49. Host cell genetically modified for the production of sialylated oligosaccharide, wherein the host cell comprises at least one nucleic acid sequence coding for an enzyme for sialylated oligosaccharide synthesis and wherein said cell is genetically modified for i) overexpression of an endogenous membrane protein, ii) expression or overexpression of a homologous membrane protein, and/or iii) expression or overexpression of a heterologous membrane protein, wherein said membrane protein comprises
i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08116, 08JJA, 05FDX, 05EGG, 08JN3, 08N1B, 05IDI, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 0500; or
ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
iv) an amino acid sequence encoding a Sugar Efflux Transporter, preferably said membrane protein is an MFS transporter comprising the conserved domain L[FY]AxNR[HN]Y (SEQ ID NO 98), wherein x can be any distinct amino acid.

50. Cell according to claim 49, wherein
i) when said membrane protein is a siderophore exporter, said membrane protein is selected from SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 or functional homolog or functional fragment of any one of the above membrane protein or a sequence having at least 80% sequence identity to any one of said SEQ ID NOs 9, 4, 6, 11, 13, 15, 20, 37, 38, 39, 40, 41, 42, 43, 44, 45, 46, 47, 48, 49, 50, 51, 52, 53, 54, 55, 56, 57, 58, 59, 60, 61, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 99, 100, 101, 102, 103, 104, 105, 106, 107, 109, 110, 111, 112, 113, 114, 115, 116, 117, 118, 119, 120, 121 or 122 and providing improved production and/or efflux of sialylated oligosaccharides;
ii) when said membrane protein is an ABC transporter, said membrane protein is selected from oppF from Escherichia coli K12 MG1655 with SEQ ID NO 18, lmrA from Lactococcus lactis subsp. lactis bv. Diacetylactis with SEQ ID NO 15, Blon_2475 from B. longum subsp. Infantis (strain ATCC 15697) with SEQ ID NO 19 or gsiA from Escherichia coli K12 MG1655 with SEQ ID NO 63, or functional homolog or functional fragment of any one of the above transporter membrane protein or a sequence having at least 80% sequence identity to any one of said SEQ ID NOs 18, 15, 19 or 63 and providing improved production and/or efflux of sialylated oligosaccharides;
iii) when said membrane protein is an MFS transporter, said membrane protein is selected from SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 or functional homolog or functional fragment of any one of the above transporter membrane protein or a sequence having at least 80% sequence identity to any one of said SEQ ID NOs 4, 5, 6, 7, 8, 9, 10, 11, 12, 13, 14, 20, 21, 37, 38, 39, 40, 41, 42, 43, 44, 45, 46, 47, 48, 49, 50, 51, 52, 53, 54, 55, 56, 57, 58, 59, 60, 61, 100, 106, 107, 108, 111, 113, 116, 117, 118, 119, 121 or 122 and providing improved production and/or efflux of sialylated oligosaccharides;
iv) when said membrane protein is a Sugar Efflux Transporter, said membrane protein is selected from SEQ ID NOs 2, 1, 3, 16, 17 or 62, or functional homolog or functional fragment of any one of the above transporter membrane protein or a sequence having at least 80% sequence identity to any one of said SEQ ID NOs 2, 1, 3, 16, 17 or 62 and providing improved production and/or efflux of sialylated oligosaccharides.

51. Cell according to claim 49, wherein said cell is selected from the group consisting of microorganism, plant, or animal cells, preferably said microorganism is a bacterium, fungus or a yeast, preferably said plant is a rice, cotton, rapeseed, soy, maize or corn plant, preferably said animal is an insect, fish, bird or non-human mammal; preferably the cell is an Escherichia coli cell.

52. Cell according to claim 49, wherein the cell comprises a catabolic pathway for selected mono-, di- or oligosaccharides which is at least partially inactivated, the mono-, di-, or oligosaccharides being involved in and/or required for the synthesis of sialylated oligosaccharide.

53. Cell according to claim 49, wherein said sialylated oligosaccharide is 6′-sialyllactose, 3′-sialyllactose, 3-fucosyl-3′-sialyllactose (3′-O-sialyl-3-O-fucosyllactose, FSL), 2′-fucosyl-3′-sialyllactose, 2′-fucosyl-6′-sialyllactose, 3,6-disialyllactose, 6,6′-disialyllactose, sialyllacto-N-tetraose a (LSTa), fucosyl-LSTa (FLSTa), sialyllacto-N-tetraose b (LSTb), fucosyl-LSTb (FLSTb), sialyllacto-N-neotetraose c (LSTc), fucosyl-LSTc (FLSTc), sialyllacto-N-neotetraose d (LSTd), fucosyl-LSTd (FLSTd), sialyl-lacto-N-hexaose (SLNH), sialyl-lacto-N-neohexaose I (SLNH-I), sialyl-lacto-N-neohexaose II (SLNH-II), disialyl-lacto-N-tetraose (DS-LNT), 6′-O-sialylated-lacto-N-neotetraose, 3′-O-sialylated-lacto-N-tetraose, 6′-sialylN-acetyllactosamine, 3′-sialylN-acetyllactosamine, 3-fucosyl-3′-sialylN-acetyllactosamine (3′-O-sialyl-3-O-fucosyl-N-acetyllactosamine), 3,6-disialylN-acetyllactosamine, 6,6′-disialyl-Nacetyllactosamine, 2′-fucosyl-3′-sialylN-acetyllactosamine, 2′-fucosyl-6′-sialyl-N-acetyllactosamine, 6′-sialyl-LactoNbiose, 3′-sialyl-LactoNbiose, 4-fucosyl-3′-sialyl-LactoNbiose (3′-O-sialyl-4-O-fucosyl-LactoNbiose), 3′,6′-disialyl-LactoNbiose, 6,6′-disialyl-LactoNbiose, 2′-fucosyl-3′-sialyl-LactoNbiose, 2′-fucosyl-6′-sialyl-LactoNbiose.

54. Cell according to claim 49, characterized in that it is further transformed to comprise at least one nucleic acid sequence coding for a protein facilitating or promoting the import of substrate required for oligosaccharide synthesis, wherein the protein is selected from the group consisting of lactose transporter, fucose transporter, sialic acid transporter, galactose transporter, mannose transporter, N-acetylglucosamine transporter, N-acetylgalactosamine transporter, ABC-transporter, transporter for a nucleotide-activated sugar and transporter for a nucleobase, nucleoside or nucleotide.

55. Cell according to claim 49, characterized in that it is further transformed to comprise at least one nucleic acid sequence coding for a protein selected from the group consisting of nucleotidyltransferase, guanylyltransferase, uridylyltransferase, Fkp, L-fucose kinase, fucose-1-phosphate guanylyltransferase, CMP-sialic acid synthetase, galactose kinase, galactose-1-phosphate uridylyltransferase, glucose kinase, glucose-1-phosphate uridylyltransferase, mannose kinase, mannose-1-phosphate guanylyltransferase, GDP-4-keto-6-deoxy-D-mannose reductase, glucosamine kinase, glucosamine-phosphate acetyltransferase, N-acetyl-glucosamin-phosphate uridylyltransferase, UDP-N-acetylglucosamine 4-epimerase, UDP-N-acetyl-glucosamine 2-epimerase, cytidyltransferase, fructose-6-P-aminotransferase, glucosamine-6-P-aminotransferase, phosphatase, N-acetylglucosamine-2-epimerase, sialic acid synthase, ManNAc kinase, sialic acid synthetase, sialic acid phosphatase.

56. A bacterial cell for the production of sialyllactose, the cell being transformed to comprise at least one nucleic acid sequence coding for a sialyltransferase, characterized in that:
the cell in addition is transformed to comprise at least one nucleic acid sequence coding for a membrane protein wherein said membrane protein comprises
i) an amino acid sequence encoding a siderophore exporter, preferably a siderophore exporter as part of any one of NOG families COG0477, 0ZVQG, 0ZPI7, 0ZVXV, 0XNN3, COG3182, 0ZW7F, 0XP7I, 0ZVCH, 0XQZX, 0XNQK, 0ZVYD, COG2271, 0XNNX, 0ZZWT, COG2814, 0ZITE, 0ZVC8, 0XT98, 0XNQ6, 0YAQV, 0ZVQA, COG2211, COG3104, 1269U, 0ZW8Z, COG1132, COG1173, COG0842, COG4615, COG0577, COG2274, COG4618, COG4172, COG5265, COG1136, 0XPIZ, COG0444, COG4779, COG4606, COG0601, COG1108, COG3182, COG4214, COG4605, COG2409, COG0841, COG3696, COG0845, COG1033, COG0534, 0Y3TF, COG2244, 0XPYW, COG2223 or bactNOG families 05E8G, 08HFG, 089VA, 07TNI, 05C0R, 07Y9F, 05CSH, 05QRD, 05EDF, 05C6X, 08NGX, 05C2C, 07FU4, 07U9Z, 080SS, 07SFI, 05EYM, 05C57, 08E7F, 07QF7, 05CSP, 07UZE, 07VHC, 08EFJ, 05CT4, 05FCD, 07YDJ, 08MMW, 08TKV, 07XMP, 05BZ1, 05IBP, 05CK8, 05IUH, 05D6C, 08E0J, 08116, 08JJA, 05FDX, 05EGG, 08JN3, 08N1B, 051DI, 08ITX, 05TVJ, 05DHS, 05CM4, 07RUJ, 05EYF, 07R13, 05BZS, 08IJF, 05UQX, 05C3S, 07U3M, 07R73, 07T1S, 07TJ5, 07XCD, 05DJC, 07RBJ, 05CXP; or
ii) an amino acid sequence encoding an ABC transporter comprising a) a conserved domain GxSGxGKST (SEQ ID NO 94) and b) a conserved domain SGGQxQRxxxxRAxxxxPK (SEQ ID NO 95) wherein x can be any distinct amino acid; or
iii) an amino acid sequence encoding an MFS transporter comprising a) a conserved domain [AGMS]x[FLMVY]x[DGKNQR]xx[EGST][PRTVY][KR]x[GILMV] (SEQ ID NO 96) and b) a conserved domain [LRST]xxx[AG][AFILV] (SEQ ID NO 97), wherein x can be any distinct amino acid; or
iv) an amino acid sequence encoding a Sugar Efflux Transporter, preferably said membrane protein is an MFS transporter comprising the conserved domain L[FY]AxNR[HN]Y (SEQ ID NO: 98), wherein x can be any distinct amino acid.

57. Bacterial cell according to claim 56, characterized in that the cell is an Escherichia coli cell.


SEQUENCE LISTING (122 sequences)

SEQ ID NO: 1 - protein, 392 aa, Escherichia coli K12 MG1655
MIWIMTMARRMNGVYAAFMLVAFMMGVAGALQAPTLSLFLSREVGAQPFWIGLFYTVNAIAGIGVSLWLAKRSDSQGDRRKLIIFCCLMAIGNALLFAFNRHYLTLITCGVLLASLANTAMPQLFALAREYADNSAREVVMFSSVMRAQLSLAWVIGPPLAFMLALNYGFTVMFSIAAGIFTLSLVLIAFMLPSVARVELPSENALSMQGGWQDSNVRMLFVASTLMWTCNTMYIIDMPLWISSELGLPDKLAGFLMGTAAGLEIPAMILAGYYVKRYGKRRMMVIAVAAGVLFYTGLIFFNSRMALMTLQLFNAVFIGIVAGIGMLWFQDLMPGRAGAATTLFTNSISTGVILAGVIQGAIAQSWGHFAVYWVIAVISVVALFLTAKVKDV

SEQ ID NO: 2 - protein, 393 aa, Escherichia coli K12 MG1655
MHNSPAVSSAKSFDLTSTAFLIVAFLTGIAGALQTPTLSIFLTDEVHARPAMVGFFFTGSAVIGILVSQFLAGRSDKRGDRKSLIVFCCLLGVLACTLFAWNRNYFVLLFVGVFLSSFGSTANPQMFALAREHADKTGREAVMFSSFLRAQVSLAWVIGPPLAYALAMGFSFTVMYLSAAVAFIVCGVMVWLFLPSMRKELPLATGTIEAPRRNRRDTLLLFVICTLMWGSNSLYIINMPLFIINELHLPEKLAGVMMGTAAGLEIPTMLIAGYFAKRLGKRFLMRVAAVGGVCFYAGMLMAHSPVILLGLQLLNAIFIGILGGIGMLYFQDLMPGQAGSATTLYTNTSRVGWIIAGSVAGIVAEIWNYHAVFWFAMVMIIATLFCLLRIKDV

SEQ ID NO: 3 - protein, 394 aa, Escherichia coli K12 MG1655
MQKTATTPSKILDLTAAAFLLVAFLTGIAGALQTPTLSIFLADELKARPIMVGFFFTGSAIMGILVSQFLARHSDKQGDRKLLILLCCLFGVLACTLFAWNRNYFILLSTGVLLSSFASTANPQMFALAREHADRTGRETVMFSTFLRAQISLAWVIGPPLAYELAMGFSFKVMYLTAAIAFVVCGLIVWLFLPSIQRNIPVVTQPVEILPSTHRKRDTRLLFVVCSMMWAANNLYMINMPLFIIDELHLTDKLTGEMIGIAAGLEIPMMLIAGYYMKRIGKRLLMLIAIVSGMCFYASVLMATTPAVELELQILNAIFLGILCGIGMLYFQDLMPEKIGSATTLYANTSRVGWIIAGSVDGIMVEIWSYHALFWLAIGMLGIAMICLLFIKDI

SEQ ID NO: 4 - protein, 410 aa, Escherichia coli K12 MG1655
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNGRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHEG

SEQ ID NO: 5 - protein, 417 aa, Escherichia coli K12 MG1655
MSRTTTVDGAPASDTDKQSISQPNQFIKRGTPQFMRVTLALFSAGLATFALLYCVQPILPVLSQEFGLTPANSSISLSISTAMLAIGLLFTGPLSDAIGRKPVMVTALLLASICTLLSTMMTSWHGILIMRALIGLSLSGVAAVGMTYLSEEIHPSFVAFSMGLYISGNSIGGMSGRLISGVFTDFFNWRIALAAIGCFALASALMFWKILPESRHFRPTSLRPKTLFINFRLHWRDRGLPLLFAEGFLLMGSFVTLFNYIGYRLMLSPWHVSQAVVGLLSLAYLTGTWSSPKAGTMTTRYGRGPVMLFSTGVMLFGLLMTLFSSLWLIFAGMLLFSAGFFAAHSVASSWIGPRAKRAKGQASSLYLFSYYLGSSIAGTLGGVFWHNYGWNGVGAFIALMLVIALLVGTRLHRRLHA

SEQ ID NO: 6 - protein, 471 aa, Escherichia coli K12 MG1655
MTDLPDSTRWQLWIVAFGFFMQSLDTTIVNTALPSMAQSLGESPLHMHMVIVSYVLTVAVMLPASGWLADKVGVRNIFFTAIVLFTLGSLFCALSGTLNELLLARALQGVGGAMMVPVGRLTVMKIVPREQYMAAMTFVTLPGQVGPLLGPALGGLLVEYASWHWIFLINIPVGIIGAIATLLLMPNYTMQTRRFDLSGFLLLAVGMAVLTLALDGSKGTGLSPLTIAGLVAVGVVALVLYLLHARNNNRALFSLKLFRTRTFSLGLAGSFAGRIGSGMLPFMTPVFLQIGLGFSPFHAGLMMIPMVLGSMGMKRIVVQVVNRFGYRRVLVATTLGLSLVTLLFMTTALLGWYYVLPFVLFLQGMVNSTRFSSMNTLTLKDLPDNLASSGNSLLSMIMQLSMSIGVTIAGLLLGLFGSQHVSVDSGTTQTVFMYTWLSMALIIALPAFIFARVPNDTHQNVAISRRKRSAQ

SEQ ID NO: 7 - protein, 405 aa, Escherichia coli K12 MG1655
MPEPVAEPALNGLRLNLRIVSIVMFNFASYLTIGLPLAVLPGYVHDVMGFSAFWAGLVISLQYFATLLSRPHAGRYADSLGPKKIVVFGLCGCFLSGLGYLTAGLTASLPVISLLLLCLGRVILGIGQSFAGTGSTLWGVGVVGSLHIGRVISWNGIVTYGAMAMGAPLGVVFYHWGGLQALALIIMGVALVAILLAIPRPTVKASKGKPLPFRAVLGRVWLYGMALALASAGFGVIATFITLFYDAKGWDGAAFALTLFSCAFVGTRLLFPNGINRIGGLNVAMICFSVEIIGLLLVGVATMPWMAKIGVLLAGAGFSLVFPALGVVAVKAVPQQNQGAALATYTVFMDLSLGVTGPLAGLVMSWAGVPVIYLAAAGLVAIALLLTWRLKKRPPEHVPEAASSS

SEQ ID NO: 8 - protein, 403 aa, Escherichia coli K12 MG1655
MQPGKRFLVWLAGLSVLGFLATDMYLPAFAAIQADLQTPASAVSASLSLFLAGFAAAQLLWGPLSDRYGRKPVLLIGLTIFALGSLGMLWVENAATLLVLRFVQAVGVCAAAVIWQALVTDYYPSQKVNRIFAAIMPLVGLSPALAPLLGSWLLVHFSWQAIFATLFAITVVLILPIFWLKPTTKARNNSQDGLTFTDLLRSKTYRGNVLIYAACSASFFAWLTGSPFILSEMGYSPAVIGLSYVPQTIAFLIGGYGCRAALQKWQGKQLLPWLLVLFAVSVIATWAAGFISHVSLVEILIPFCVMAIANGAIYPIVVAQALRPFPHATGRAAALQNTLQLGLCFLASLVVSWLISISTPLLTTTSVMLSTVVLVALGYMMQRCEEVGCQNHGNAEVAHSESH

SEQ ID NO: 9 - protein, 416 aa, Escherichia coli K12 MG1655
MNKQSWLLNLSLLKTHPAFRAVFLARFISIVSLGLLGVAVPVQIQMMTHSTWQVGLSVTLTGGAMFVGLMVGGVLADRYERKKVILLARGTCGIGFIGLCLNALLPEPSLLAIYLLGLWDGFFASLGVTALLAATPALVGRENLMQAGAITMLTVRLGSVISPMIGGLLLATGGVAWNYGLAAAGTFITLLPLLSLPALPPPPQPREHPLKSLLAGFRFLLASPLVGGIALLGGLLTMASAVRVLYPALADNWQMSAAQIGFLYAAIPLGAAIGALTSGKLAHSARPGLLMLLSTLGSFLAIGLFGLMPMWILGVVCLALFGWLSAVSSLLQYTMLQTQTPEAMLGRINGLWTAQNVTGDAIGAALLGGLGAMMTPVASASASGFGLLIIGVLLLLVLVELRHFRQTPPQVTASDS

SEQ ID NO: 10 - protein, 403 aa, Escherichia coli K12 MG1655
MSTRTPSSSSSRLMLTIGLCFLVALMEGLDLQAAGIAAGGIAQAFALDKMQMGWIFSAGILGLLPGALVGGMLADRYGRKRILIGSVALFGLFSLATAIAWDFPSLVFARLMTGVGLGAALPNLIALTSEAAGPRFRGTAVSLMYCGVPIGAALAATLGFAGANLAWQTVFWVGGVVPLILVPLLMRWLPESAVFAGEKQSAPPLRALFAPETATATLLLWLCYFFTLLVVYMLINWLPLLLVEQGFQPSQAAGVMFALQMGAASGTLMLGALMDKLRPVTMSLLIYSGMLASLLALGTVSSFNGMLLAGFVAGLFATGGQSVLYALAPLFYSSQIRATGVGTAVAVGRLGAMSGPLLAGKMLALGTGTVGVMAASAPGILVAGLAVFILMSRRSRIQPCADA

SEQ ID NO: 11 - protein, 457 aa, Escherichia coli K12 MG1655
MPKVQADGLPLPQRYGAILTIVIGISMAVLDGAIANVALPTIATDLHATPASSIWVVNAYQIAIVISLLSFSFLGDMFGYRRIYKCGLVVFLLSSLFCALSDSLQMLTLARVIQGFGGAALMSVNTALIRLIYPQRFLGRGMGINSFIVAVSSAAGPTIAAAILSIASWKWLFLINVPLGIIALLLAMRFLPPNGSRASKPRFDLPSAVMNALTFGLLITALSGFAQGQSLTLIAAELVVMVVVGIFFIRRQLSLPVPLLPVDLLRIPLFSLSICTSVCSFCAQMLAMVSLPFYLQTVLGRSEVETGLLLTPWPLATMVMAPLAGYLIERVHAGLLGALGLFIMAAGLFSLVLLPASPADINIIWPMILCGAGFGLFQSPNNHTIITSAPRERSGGASGMLGTARLLGQSSGAALVALMLNQFGDNGTHVSLMAAAILAVIAACVSGLRITQPRSRA

SEQ ID NO: 12 - protein, 405 aa, Escherichia coli K12 MG1655
MATAWYKQVNPPQRKALFSAWLGYVFDGFDFMMIFYILHIIKADLGITDIQATLIGTVAFIARPIGGGFFGAMADKYGRKPMMMWAIFIYSVGTGLSGIATNLYMLAVCRFIVGLGMSGEYACASTYAVESWPKNLQSKASAFLVSGFSVGNIIAAQIIPQFAEVYGWRNSFFIGLLPVLLVLWIRKSAPESQEWIEDKYKDKSTFLSVFRKPHLSISMIVFLVCFCLFGANWPINGLLPSYLADNGVNTVVISTLMTIAGLGTLTGTIFFGFVGDKIGVKKAFVVGLITSFIFLCPLFFISVKNSSLIGLCLFGLMFTNLGIAGLVPKFIYDYFPTKLRGLGTGLIYNLGATGGMAAPVLATYISGYYGLGVSLFIVTVAFSALLILLVGFDIPGKIYKLSVAK

SEQ ID NO: 13 - protein, 396 aa, Escherichia coli K12 MG1655
MTTRQHSSFAIVFILGLLAMLMPLSIDMYLPALPVISAQFGVPAGSTQMTLSTYILGFALGQLIYGPMADSFGRKPVVLGGTLVFAAAAVACALANTIDQLIVMRFFHGLAAAAASVVINALMRDIYPKEEFSRMMSFVMLVTTIAPLMAPIVGGWVLVWLSWHYIFWILALAAILASAMIFFLIKETLPPERRQPFHIRTTIGNFAALFRHKRVLSYMLASGFSFAGMFSFLSAGPFVYIEINHVAPENFGYYFALNIVFLFVMTIFNSRFVRRIGALNMFRSGLWIQFIMAAWMVISALLGLGFWSLVVGVAAFVGCVSMVSSNAMAVILDEFPHMAGTASSLAGTFRFGIGAIVGALLSLATFNSAWPMIWSIAFCATSSILFCLYASRPKKR

SEQ ID NO: 14 - protein, 438 aa, Escherichia coli K12 MG1655
MGNTSIQTQSYRAVDKDAGQSRSYIIPFALLCSLFFLWAVANNLNDILLPQFQQAFTLTNFQAGLIQSAFYFGYFIIPIPAGILMKKLSYKAGIITGLFLYALGAALFWPAAEIMNYTLFLVGLFIIAAGLGCLETAANPFVTVLGPESSGHFRLNLAQTFNSFGAIIAVVFGQSLILSNVPHQSQDVLDKMSPEQLSAYKHSLVLSVQTPYMIIVAIVLLVALLIMLTKFPALQSDNHSDAKQGSFSASLSRLARIRHWRWAVLAQFCYVGAQTACWSYLIRYAVEEIPGMTAGFAANYLTGTMVCFFIGRFTGTWLISRFAPHKVLAAYALIAMALCLISAFAGGHVGLIALTLCSAFMSIQYPTIFSLGIKNLGQDTKYGSSFIVMTIIGGGIVTPVMGFVSDAAGNIPTAELIPALCFAVIFIFARFRSQTATN

SEQ ID NO: 15 - protein, 584 aa, Lactococcus lactis subsp. lactis bv. diacetylactis
MANRIEGKAVDKTSIKHFIKLIRAAKPRYLFFIIGILAGIVGTLIQLQVPKMVQPLVNSFGHGVNGGKVALVIALYIGSAAVSAIAAIVLGIFGESVVKNLRTRVWDKMIHLPVKYFDEVKTGEMSSRLANDTTQVKNLIANSIPQAFTSILLLVGSIVFMLQMQWRLTLAMIIAVPVVMLIMFPIMTFGQKIGRTRQDSLANFQGIASESLSEIRLVKSSNAEKQASKKAENDVNALYKIGVKEAIFDGLMSPVMMLSMMLMIFGLLAYGIYLISTGVMSLGTLLGMMMYLMNLIGAVPTVATFFTELAKASGSTGRLTELLDEEQEVLHQGESLDLEGKTLSARHVDFAYDDSEQILRDISFEAQPNSIIAFAGPSGGGKSTIFSLLERFYQPTAGEITIDGQPIDNISLENWRSQIGFVSQDSAIMAGTIRENLTYGLEGDYTDEDLWQVLDLAFARSFVENMPDQLNTEVGERGVKISGGQRQRLAIARAFLRNPKILMLDEATASLDSESESMVQKALDSLMKGRTTLVIAHRLSTIVDADKIYFIEKGQITGSGKHNELVATHPLYAKYVSEQLTVGQ

SEQ ID NO: 16 - protein, 339 aa, Pectobacterium carotovorum subsp carotovorum PCC21
MFYTGSAVIGIVVSQMLATRSDRQGDRKSLIFVCCLLGALACMLFAWNRNYFILLFIGVLLSSFGSTANPQLFALAREHADKTGREAAMFSSILRAQISLAWVVGPPIAFALALGFGFTTMYLTAAVVFILCGILVKLFLPSMPKAVEKTTSTLESPRRNRRDTLLLFVACTLMWTCNGIYLINMPLYLVHELHLPEKLAGIMMGVAAGLEIPVMLIAGYVAKRFGKRFLMRLAVASGLLFFGGLLVLDGEISLLALQVLNAIFIGILAGIGMLYFQDLMPGQAGAATTLFTNTTRVGWIISGSLAGIVAEIWNYHAVFFFALLMIAGSIYCMWRIKDA

SEQ ID NO: 17 - protein, 430 aa, Pectobacterium carotovorum subsp carotovorum PCC21
MAEAVLTKNNETRFLFHYKPEYYVALDSLCLFSKRYQFMFTPANTTRRPLDLTSSAFLVIAFLTGTAGALQLPTLSLFLSSEVQARPFLVGMFYTGSAVIGIVVSQMLATRSDRQGDRKSLIFVCCLLGALACMLFAWNRNYFILLFIGVLLSSFGSTANPQLFALAREHADKTGREAAMFSSILRAQISLAWVVGPPIAFALALGFGFTTMYLTAAVVFILCGILVKLFLPSMPKAVEKTTSTLESPRRNRRDTLLLFVACTLMWTCNGIYLINMPLYLVHELHLPEKLAGIMMGVAAGLEIPVMLIAGYVAKRFGKRFLMRLAVASGLLFFGGLLVLDGEISLLALQVLNAIFIGILAGIGMLYFQDLMPGQAGAATTLFTNTTRVGWIISGSLAGIVAEIWNYHAVFFFALLMIAGSIYCMWRIKDA

SEQ ID NO: 18 - protein, 334 aa, Escherichia coli K12 MG1655
MNAVTEGRKVLLEIADLKVHFEIKDGKQWFWQPPKTLKAVDGVTLRLYEGETLGVVGESGCGKSTFARAIIGLVKATDGHVAWLGKELLGMKPDEWRAVRSDIQMIFQDPLASLNPRMTIGEIIAEPLRTYHPKMSRQEVRERVKAMMLKVGLLPNLINRYPHEFSGGQCQRIGIARALILEPKLIICDEPVSALDVSIQAQVVNLLQQLQREMGLSLIFIAHDLAVVKHISDRVLVMYLGHAVELGTYDEVYHNPLHPYTRALMSAVPIPDPDLEKNKTIQLLEGELPSPINPPSGCVFRTRCPIAGPECAKTRPVLEGSFRHSVSCLKVDPL

SEQ ID NO: 19 - protein, 375 aa, Bifidobacterium longum subsp. infantis (strain ATCC 15697)
MAEVVFDHVTRIYPGNDKPSVDDLNLDIKDGEFLVLVGPSGCGKSTTLRMLAGLEEVNKGRILIGGKDVTTMQPKDRDIAMVFQNYALYPHMTVADNMGFALKIAGTPKDEIRKRVEKAAEILDLTEYLDRKPKALSGGQRQRVAMGRAIVREPKVFLMDEPLSNLDAKLRVQTRTQIAALQRQLGVTTLYVTHDQTEALTMGDRIAVIKLGILQQVGAPTELYDRPANVFVAGFIGSPSMNLNTHPVVNGKAKIGEDTVDLPAEAVNKLTAEDNGQIVVGFRPEDAGLAPVDDPNAFSLKVVNVEDLGSDGYIYGTIVTDGSAAEASQVMSDQNKLTTIRVNPRALPKVGATVKIKIDPAKMHLFAPSTELRLN

SEQ ID NO: 20 - protein, 405 aa, Bifidobacterium longum subsp. infantis (strain ATCC 15697)
MALDVGKALKSKTLVVGVLSMSLLMSASNAVSGTIPAMKEAFSDYSAANVELLTTVPTIGSMVGTALTGLFANAIGRKKIAMAGFLISAVTGVIPAFFPYYWPILISRMFFGFGSALFVTLSVSYITDLYDGDMQRKLLGWRQAVGNLGDVVLLFVASLLITINWQSTYLIFFLLFVPMVLVGMFIPKEFDNFSIRSALVDDEGHVVDKSASQKQTTNWQVLWLAFIFLVVSMLYNVMSIKLASYVVDEGIGSASLATLIFSFLVVATILSGVLFDKVAKVTKRLTVTISEVVIGICFIATALTKNVPLMFALVLIAGFAWGIINPALTARFVDYSPAHSMNLTTSIVIIGINIGCLISPYFFALTASIFGNSSAGFAIIVGGALYLVMVVIELITLKVDKKLTV

SEQ ID NO: 21 - protein, 401 aa, Bifidobacterium longum subsp. infantis (strain ATCC 15697)
MIQRYSQGTSARRIFPILLIAEGLCLSGMSVDLTITSLTGSSLAPAPWMATFPIACIFIGTVIGTPAMGRLVGRFGYRQVFVFGALLAVLGGISSATALRFHQFPLLCIGTFCVGIYQSGTNYYRYAAADSMPGKESKAINGILSAGAIASIIGPVLATFFGTATNIEYEGSYYVVSVLAACAVIVLLALPKIQSPTPAHMRDKATASTTPNYPALLTRPRFVLGATISFVASFVMVLVMSGAPIVLESTFSQNAQTRMVAMQLHMVGMYLPTLFAIFIFHKKNSEMAQALTGLIIGMLGTVVALAASASYAVTLDLLLIGISWSLCYAAGSALLTKSYTESERSWARGVGELAPVLGQVLGSVLAGVLLAAGWKTVFVTVLAMIVCALIAMAGYRNKIKS

SEQ ID NO: 22 - protein, 417 aa, Escherichia coli K12 MG1655 [sequence omitted]

SEQ ID NO: 23 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 24 - protein, 415 aa, Escherichia coli W
MALNIPFRNAYYRFASSYSFLFFISWSLWWSLYAIWLKGHLGLTGTELGTLYSVNQFTSILFMMFYGIVQDKLGLKKPLIWCMSFILVLTGPFMIYVYEPLLQSNFSVGLILGALFFGLGYLAGCGLLDSFTEKMARNFHFEYGTARAWGSFGYAIGAFFAGIFFSISPHINFWLVSLFGAVFMMINMRFKDKDHQCVAADAGGVKKEDFIAVFKDRNFWVFVIFIVGTWSFYNIFDQQLFPVFYSGLFESHDVGTRLYGYLNSFQVVLEALCMAIIPFFVNRVGPKNALLIGVVIMALRILSCALFVNPWIISLVKLLHAIEVPLCVISVFKYSVANFDKRLSSTIFLIGFQIASSLGIVLLSTPTGILFDHAGYQTVFFAISGIVCLMLLFGIFFLSKKREQIVMETPVPSAI

SEQ ID NO: 25 - protein, 504 aa, Bifidobacterium adolescentis [sequence omitted]

SEQ ID NO: 26 - protein, 301 aa, Zymomonas mobilis
MKNDKKIYGCIEGGGTKFMLALIDSDRKMLAVERVPTTTPEETLGKRVEFFKKALPQYADSFASFGIASFGPLCLDRKSPKWGYITNTPKPFWPNTDVVTPFKEAFGCPVEIDTDVNGAALAENFWGASKGTHTSVYVTVGTGFGGGVLIDGKPIHGLAHPEMGHGIPIRHPDDRDFEGCCPYHGGCYEGLASGTAIRKRWGKALNEMEPAEFEKAREIIAFYLAHFNVTLQAFISPERIVFGGGVMHVDGMLASVRRQTAEIANSYFEGADFEKIIVLPGLGDQAGMMGAFALALAAENK

SEQ ID NO: 27 - protein, 609 aa, Escherichia coli K12 MG1655
MCGIVGAIAQRDVAEILLEGLRRLEYRGYDSAGLAVVDTEGHMTRLRRLGKVQMLAQAAEEHPLHGGTGIAHTRWATHGEPSEVNAHPHVSEHIVVVHNGIIENHEPLREELKARGYTFVSETDTEVIAHLVNWELKQGGTLREAVLRAIPQLRGAYGTVIMDSRHPDTLLAARSGSPLVIGLGMGENFIASDQLALLPVTRRFIFLEEGDIAEITRRSVNIFDKTGAEVKRQDIESNLQYDAGDKGIYCHYMQKEIYEQPNAIKNTLTGRISHGQVDLSELGPNADELLSKVEHIQILACGTSYNSGMVSRYWFESLAGIPCDVEIASEFRYRKSAVRRNSLMITLSQSGETADTLAGLRLSKELGYLGSLAICNVPGSSLVRESDLALMTNAGTEIGVASTKAFTTQLTVLLMLVAKLSRLKGLDASIEHDIVHGLQALPSRIEQMLSQDKRIEALAEDFSDKHHALFLSRGDQYPIALEGALKLKEISYIHAEAYAAGELKHGPLALIDADMPVIVVAPNNELLEKLKSNIEEVRARGGQLYVFADQDAGFVSSDNMHIIEMPHVEEVIAPIFYTVPLQLLAYHVALIKGTDVDQPRNLAKSVTVE

SEQ ID NO: 28 - protein, 159 aa, Saccharomyces cerevisiae
MSLPDGFYIRRMEEGDLEQVTETLKVLTTVGTITPESFSKLIKYWNEATVWNDNEDKKIMQYNPMVIVDKRTETVAATGNIIIERKIIHELGLCGHIEDIAVNSKYQGQGLGKLLIDQLVTIGFDYGCYKIILDCDEKNVKFYEKCGFSNAGVEMQIRK

SEQ ID NO: 29 - protein, 421 aa, Bacteroides ovatus
MDSKNNIGHSADISLTAELPIPIYNGNTIMDFKKLASLYKDELLDNVLPFWLEHSQDHEYGGYFTCLDREGKVFDTDKFIWLQSREVWMFSMLYNKVEKRQEWLDCAIQGGEFLKKYGHDGNYNWYFSLDRSGRPLVEPYNIFSYTFATMAFGQLSLTTGNQEYADIAKKTFDIILSKVDNPKGRWNKLHPGTRNLKNFALPMILCNLALEIEHLLDETYLRETMDTCIHEVMEVFYRPELGGIIVENVDIDGNLVDCFEGRQVTPGHAIEAMWFIMDLGKRLNRPELIEKAKETTLTMLNYGWDKQYGGIYYFMDRNGCPPQQLEWDQKLWWVHIETLISLLKGYQLTGDKKCLEWFEKVHDYTWEHFKDKEYPEWYGYLNRRGEVLLPLKGGKWKGCFHVPRGLYQCWKTLEEIKNIVS

SEQ ID NO: 30 - protein, 346 aa, Campylobacter jejuni
MKEIKIQNIIISEEKAPLVVPEIGINHNGSLELAKIMVDAAFSAGAKIIKHQTHIVEDEMSKAAKKVIPGNAKISIYEIMQKCALDYKDELALKEYTEKLGLVYLSTPFSRAGANRLEDMGVSAFKIGSGECNNYPLIKHIAAFKKPMIVSTGMNSIESIKPTVKILLDNEIPFVLMHTTNLYPTPHNLVRLNAMLELKKEFSCMVGLSDHTTDNLACLGAVVLGACVLERHFTDSMHRSGPDIVCSMDTKALKELIIQSEQMAIIRGNNESKKAAKQEQVTIDFAFASVVSIKDIKKGEVLSMDNIWVKRPGLGGISAAEFENILGKKALRDIENDAQLSYEDFA

SEQ ID NO: 31 - protein, 228 aa, Neisseria meningitidis
MEKQNIAVILARQNSKGLPLKNLRKMNGISLLGHTINAAISSKCFDRIIVSTDGGLIAEEAKNFGVEVVLRPAELASDTASSISGVIHALETIGSNSGTVTLLQPTSPLRTGAHIREAFSLFDEKIKGSVVSACPMEHHPLKTLLQINNGEYAPMRHLSDLEQPRQQLPQAFRPNGAIYINDTASLIANNCFFIAPTKLYIMSHQDSIDIDTELDLQQAENILNHKES

SEQ ID NO: 32 - protein, 391 aa, Photobacterium damselae
MTVVAPTLEVYIDHASLPSLQQLIHIIQAKDEYPSNQRFVSWKRVTVDADNANKLNIHTYPLKGNNTSPEMVAAIDEYAQSKNRLNIEFYTNTAHVFNNLPPIIQPLYNNEKVKISHISLYDDGSSEYVSLYQWKDTPNKIETLEGEVSLLANYLAGTSPDAPKGMGNRYNWHKLYDTDYYFLREDYLDVEANLHDLRDYLGSSAKQMPWDEFAKLSDSQQTLFLDIVGFDKEQLQQQYSQSPLPNFIFTGTTTWAGGETKEYYAQQQVNVINNAINETSPYYLGKDYDLFFKGHPAGGVINDIILGSFPDMINIPAKISFEVLMMTDMLPDTVAGIASSLYFTIPADKVNFIVFTSSDTITDREEALKSPLVQVMLTLGIVKEKDVLFWA

SEQ ID NO: 33 - protein, 371 aa, Neisseria meningitidis
MGLKKACLTVLCLIVFCFGIFYTFDRVNQGERNAVSLLKEKLFNEEGEPVNLIFCYTILQMKVAERIMAQHPGERFYVVLMSENRNEKYDYYFNQIKDKAERAYFFHLPYGLNKSFNFIPTMAELKVKSMLLPKVKRIYLASLEKVSIAAFLSTYPDAEIKTFDDGTGNLIQSSSYLGDEFSVNGTIKRNFARMMIGDWSIAKTRNASDEHYTIFKGLKNIMDDGRRKMTYLPLFDASELKTGDETGGTVRILLGSPDKEMKEISEKAAKNFKIQYVAPHPRQTYGLSGVTTLNSPYVIEDYILREIKKNPHTRYEIYTFFSGAALTMKDFPNVHVYALKPASLPEDYWLKPVYALFTQSGIPILTFDDKN

SEQ ID NO: 34 - protein, 348 aa, Neisseria meningitidis
MPSEAFRRHRAYRENKLQPLVSVLICAYNVEKYFAQSLAAVVNQTWCNLDILIVDDGSTDGTLAIAQRFQEQDGRIKILAQAQNSGLIPSLNIGLDELAKSGMGEYIARTDADDIAAPDWIEKIVGEMEKDRSIIAMGAWLEVLSEEKDGNRLARHHEHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTLPAGVWLDFAANGRMRRLFTLRQYFGILHRLLKNR

SEQ ID NO: 35 - protein, 275 aa, Neisseria meningitidis
MQNHVISLASAAERRAHIADTFGRHGIPFQFFDALMPSERLEQAMAELVPGLSAHPYLSGVEKACFMSHAVLWKQALDEGLPYITVFEDDVLLGEGAEKFLAEDAWLQERFDPDTAFIVRLETMFMHVLTSPSGVADYCGRAFPLLESEHWGTAGYIISRKAMRFFLDRFAALPPEGLHPVDLMMFSDFFDREGMPVCQLNPALCAQELHYAKFHDQNSALGSLIEHDRLLNRKQQRRDSPANTFKHRLIRALTKISREREKRRQRREQFIVPFQ

SEQ ID NO: 36 - protein, 265 aa, Escherichia coli K12 MG1655
MIIDEAESAESTHPVVSVILPVNKKNPFLDEAINSILSQTFSSFEIIIVANCCTDDFYNELKHKVNDKIKLIRTNIAYLPYSLNKAIDLSNGEFIARMDSDDISHPDRFTKQVDFLKNNPYVDVVGTNAIFIDDKGREINKTKLPEENLDIVKNLPYKCCIVHPSVMFRKKVIASIGGYMFSNYSEDYELWNRLSLAKIKFQNLPEYLFYYRLHEGQSTAKKNLYMVMVNDLVIKMKCFFLTGNINYLFGGIRTIASFIYCKYIK

SEQ ID NO: 37 - protein, 686 aa, Curtobacterium sp. 314Chir4.1
MSTATAPVEVQRGRQSSPAGQPGKPIMSHRQILLVIYGLMAGMFLSSLDQTIVGTAIRTIGDDLHGLDQQAWVTTAYLIASTITTPIYGKLSDVFGRRPLYIFGIAVFILGSLLSTMSTSMIMLAAFRAFQGIGAGALMSLPLAIMGDILAPRERAKYQGYFLAVFGISSVIGPLIGGLFAGASSILGITGWRWVFLINVPIGVAALLMVIAFLHLPKFGDAKAKPRIDWWGATSVIVTLVPLLLVAEQGRTWGWGSAGAIACYVIGALGLVAFLVIETLMKDDAIIPLKLFRSGVFSMATILGFLVGFAMFGAMLTIPLYLQIVTGLTPTESGFATLPMIGGLMIASIASGQIVARTGKYRIFPVIGTALVSIGYVVLTFMTIDKPLWFLMIGMFLIGLGLGQLMQSITLASQNSVQPRDMGVATSSATFFRQIGGTLGTAVLLSVLFSIMPANILHATADQKNLSSALDAALNPTVASAKANQGVMDQIWNPIVTPIKQQVQSGLDAATAQVTQATAGAPEAVQQQALTAAADKAHASVQDGKLVVDWSDSAQRTYWVDDLAPTLSKQIDKGTSDKASSSSETSDTSYLNGADSALTRPFMTGFNSSAVTVYWVGFAVILLAFILSWFFKAPPLRKSSALQEQADNDRTADDLEVQAVTAADTMGSPTAPVTGSIAVQRGSSDD

SEQ ID NO: 38 - protein, 470 aa, Citrobacter freundii MGH152
MTELPDSTRWQLWIVAFGFFMQSLDTTIVNTALPSMAKSLGESPLHMHMVIVSYVLTVAVMLPASGWLADKIGVRNIFFTAIILFTLGSLFCAWSGTLNELVMARVLQGVGGAMMVPVGRLTVMKIVPREQYMAAMTFVTLPGQIGPLLGPALGGILVEYASWHWIFLINIPVGIVGAIATLMLMPNYTMQTRRFDLSGFMMLAVGMAVLTLALDGSKGTGISHITLAALVICGVLAILLYLKHAHNNPRALFSLHLFRTHTFSLGLFGSFAGRIGSGMLPFMTPVFLQIGLGFSPFHAGLMMIPMVLGSMGMKRIVVQVVNRFGYRRVLVTTTLGLSMVSLLLMSTALLGWYYALPFILFLQGMVNSTRFSSMNTLTLKDLPDDLASSGNSLLSMIMQLSMSIGVTIAGLLLGMFGQQHIAIDSASTHTVFMYTWLCIAFLIALPAIIFARVPDDTQTNAVISRRKRST

SEQ ID NO: 39 - protein, 470 aa, Citrobacter werkmanii NBRC 105721
MTELPDSTRWQLWIVAFGFFMQSLDTTIVNTALPSMAASLGESPLHMHMVIVSYVLTVAVMLPASGWLADKVGVRNIFFTAIILFTLGSLFCAWSSTLNELVMARVLQGVGGAMMVPVGRLTVMKIVPREQYMAAMTFVTLPGQIGPLLGPALGGVLVEYASWHWIFLLNIPVGIVGAIATLMLMPNYTMQTRRFDLSGFAMLAVGMAVLTLALDGSKGTGISGITLAVLVICGALAIALYLKHAHRNPRALFSLNLFHTPTFSLGLLGSFAGRIGSGMLPFMTPVFLQIGLGFSPFHAGLMMVPMVLGSMGMKRIVVQVVNRFGYRRVLVTTTLGLSLVSLLLMTTALLGWYYALPFVLFLQGMVNSTRFSSMNTLTLKDLPDDLASSGNSLLSMIMQLSMSIGVTIAGLLLGMFGQQHIAIDSGSTHTVFMYTWLCIAFIIALPAIIFARVPNDTQTNAVISRRKRST

SEQ ID NO: 40 - protein, 470 aa, Citrobacter amalonaticus
MTELPNSTRWQLWIVAFGFFMQSLDTTIVNTALPSMAKSLGESPLHMHMVVVSYVLTVAVMLPASGWLADKIGVRNIFFTAIVLFTLGSLFCAGSGTLNELVMARVLQGVGGAMMVPVGRLTVMKIVPREQYMAAMTFVTLPGQIGPLLGPALGGILVEYASWHWIFLINLPVGIVGAIATLMLMPNYTMQTRRFDLSGFLLLAAGMAVLTIALDGSKGMGMSSFALGALVVCGVAAILLYLKHANDNPRALFSLHLFRTPTFSLGLFGSFAGRIGSGMLPFMTPVFLQIGLGFSPFHAGLMMIPMVLGSMGMKRIVVQVVNRFGYRRVLVFTTLGLSVVTLLFMSTALLGWYYVLPLVLFVQGMVNSTRFSSMNTLTLKDLPDELASSGNSLLSMIMQLSMSIGVTVAGLLLGMFAQQHMTVDSGTSHTVFMYTWLCMAFIIALPAVIFARVPNDTHKNVVIARRKRRT

SEQ ID NO: 41 - protein, 471 aa, Klebsiella oxytoca
MTDLPDSTRWQLWIVAFGFFMQSLDTTIVNTALPSMAQSLGESPLNMHMVIVSYVLTVAVMLPASGWLADKVGVRNIFFTAIVLFTLGSLFCALSATLNELLLARALQGVGGAMMVPVGRLTVMKIVPREQYMAAMTFVTLPGQVGPLLGPALGGLLVEYASWHWIFLINIPVGIIGAIATLMLMPNYTMQTRRFDLSGFLLLAVGMAVLTLALDGSKGTGLSPLAIAGLAAIGVVALVLYLLHARNNNRALFSLKLFRTRTFSLGLAGSFAGRIGSGMLPFMTPVFLQIGLGFSPFHAGLMMIPMVLGSMGMKRIVVQVVNRFGYRRVLVATTLGLSLVTLLFMTTALLGWYYVLPFVLFLQGMVNSTRFSSMNTLTLKDLPDNLASSGNSLLSMIMQLSMSIGVTIAGLLLGLFGSQHVSVDSGTTQTVFMYTWLSMAFIIALPAFIFARVSNDTHQNVAISRRKRSAQ

SEQ ID NO: 42 - protein, 468 aa, Escherichia albertii B156
MTELPDSTRWQLWIVAFGFFMQSLDTTIVNTALPSMAQSLGESPLHMHMVIVSYVLTVAVMLPASGWLADKVGVRNIFFTAIVLFTLGSLFCALSGTLNELLLARALQGVGGAMMVPVGRLTVMKIVPREQYMAAMTFVTLPGQIGPLLGPALGGLLVEYASWHWIFLINIPVGIIGAIATLMLMPNYTMQTRRFDLSGFLLLAVGMAVLTLALDGSKGTGLSPLAITVLVAVGVLAIALYLLHARNNHRALFSLKLFHTRTFSLGLAGSFAGRVGSGMLPFMTPVFLQIGLGFSPFHAGLMMIPMVLGSMGMKRIVVQVVNRFGYRRVLVATTLGLSLVTLLFMTTALLGWYYILPFVLFLQGMVNSTRFSSMNTLTLKDLPDRLASSGNSLLSMIMQLSMSIGVTIAGLLLGFFGSQHISGATQTVFMYTWLSMAFIIALPALVFARVPNDTHQNVAISRRKRSAQ

SEQ ID NO: 43 - protein, 470 aa, Salmonella enterica subsp. salamae
MTELPDSTRWQLWIVAFGFFMQSLDTTIVNTALPSMAKSLGESPLHMHMVVVSYVLTVAVMLPASGWLADKIGVRNIFFAAIVLFTLGSLFCALSGTLNQLVLARVLQGVGGAMMVPVGRLTVMKIVPRAQYMAAMTFVTLPGQVGPLLGPALGGVLVEYASWHWIFLINIPVGIVGAIATFMLMPNYTIETRRFDLPGFLLLAIGMAVLTLALDGSKSMGISPWTLAGLAAGGAAAILLYLLHAKKNSGALFSLRLFRTPTFSLGLLGSFAGRIGSGMLPFITPVFLQIGLGFSPFHAGLMMIPMVLGSMGMKRIVVQIVNRFGYRRVLVATTLGLALVSLLFMSVALLGWYYLLPLVLLLQGMVNSARFSSMNTLTLKDLPDTLASSGNSLLSMIMQLSMSIGVTIAGMLLGMFGQQHIGIDSSATHHVFLYTWLCMAVIIALPAIIFARVPNDTQQNMVISRRKRSL

SEQ ID NO: 44 - protein, 471 aa, Klebsiella pneumoniae 30684/NJST258_2
MTDLPASVRWQLWIVAFGFFMQSLDTTIVNTALPSMAKSLGESPLHMHMIIVSYVLTVAVMLPASGWLADRVGVRNIFFTAIVLFTAGSLFCAQASTLDQLVMARVLQGVGGAMMVPVGRLTVMKIVPRDQYMAAMTFVTLPGQVGPLLGPALGGVLVEYASWHWIFLINIPVGIVGAIATLCLMPNYTMQTRRFDLSGFLLLAAGMATLTLALDGQKGLGISPAWLAGLVAVGLCALLLYLWHARGNARALFSLNLFRNRTFSLGLGGSFAGRIGSGMLPFMTPVFLQIGLGFSPFHAGLMMIPMVLGSMGMKRIVVQVVNRFGYRRVLVASTLGLAAVSLLFMFSALAGWYYALPLVLFLQGMINASRFSSMNTLTLKDLPDDLASSGNSLLSMVMQLSMSIGVTIAGLLLGLYGQQHMSLDAASTHQVFLYTYLSMAAIIALPALIFSRVPDDVGSNTVLRRRNRSGS

SEQ ID NO: 45 - protein, 471 aa, Klebsiella pneumoniae kneu
MTDLPTSVRWQLWIVAFGFFMQSLDTTIVNTALPSMAKSLGESPLHMHMIIVSYVLTVAVMLPASGWLADRVGVRNIFFTAIVLFTAGSLFCAQASTLDQLVMARVLQGVGGAMMVPVGRLTVMKIVPRDQYMAAMTFVTLPGQVGPLLGPALGGVLVEYASWHWIFLINIPVGIVGAIATLCLMPNYTMQTRRFDLSGFLLLAAGMATLTLALDGQKGLGISPAWLAGLVAVGLCALLLYLWHARGNARALFSLNLFRNRTFSLGLGGSFAGRIGSGMLPFMTPVFLQIGLGFSPFHAGLMMIPMVLGSMGMKRIVVQVVNRFGYRRVLVASTLGLAAVSLLFMFSALAGWYYALPLVLFLQGMINSSRFSSMNTLTLKDLPDDLASSGNSLLSMVMQLSMSIGVTIAGLLLGLYGQQHMSLDAASTHQVFLYTYLSMAAIIALPALIFSRVPDDVGSNTVLRRRNRSGS

SEQ ID NO: 46 - protein, 471 aa, Pseudocitrobacter faecalis
MTELPASVRWQLWIVAFGFFMQSLDTTIVNTALPSMAASLGESPLHMHMVVVSYVLTVAVMLPASGWLADRVGVRNIFFTAIVLFTAGSLFCSLASTLNELLMARVLQGVGGAMMVPVGRLTVMKIVPRSQYMAAMTFVTLPGQVGPLLGPALGGVLVEYASWHWIFLINIPVGIIGGIATLMLMPNYTLQTRRFDLSGFIVLALGMAVLTLALDGQKGLGISSLTLAILVAVGVASILFYLWHARGNDNALFSLKLFRTPTFSLGLAGSFAGRIGSGMLPFMTPVFLQIGLGFSPFHAGLMMIPMVLGSMGMKRIVVQVVNRFGYRNVLVTTTIGLALVSLLFMATALAGWYYALPLVLFIQGMVNSSRFSSMNTLTLKDLPDEHASSGNSLLSMIMQLSMSIGVTVAGLLLGMFGQQHVAADSAVTHHVFMYTYLCMALIIALPALIFARVPDDVTKNVVIARRKRSEQ

SEQ ID NO: 47 - protein, 411 aa, Yokenella regensburgei ATCC43003
MQNHTLSGKRLGRQALLFPLCLVLYEFSTYIGNDMIQPGMLAVVEQYQAGIEWVPTSMTAYLAGGIFLQWLLGPLSDRIGRRPVMLTGVVWFIVTCLATLLAQNIEQFTFLRFLQGVSLCFIGAVGYAAIQESFEEAVCIKITALMANVALIAPLLGPLVGAAWIHFAPWETMFVLFAALAAISFFGLQRAMPETATRIGEKLSLKELGRDYSAVLKNLRFVAGALATGFVSLPLLAWIAQSPVIIMSGEHASSYEYGMLQVPIFGALIIGNLVLARLTSRRSVRSLIIMGGWPIVAGLILAAAATVVSSHAYLWMTAGLSVYAFGIGLANAGLVRLTLFASDMSKGTVAAAMGMLQMMIFTVGIEVSKHAYLLGGNGLFSLFNLAGGLVWLVLIVYFLKDKTVGASTKPD

SEQ ID NO: 48 - protein, 410 aa, Cronobacter muytjensii
MQTHANRTGRLGRQALLFPLCLVLYEFSTYIGNDMIQPGMLAVVEQYQAGVEWVPTSMTAYLAGGMFLQWLLGPLSDRIGRRPVMLAGVVWFIVTCLATLLAQTIEQFTVLRFLQGISLCFIGAVGYAAIQESFEEAVCIKITALMANVALIAPLLGPLVGAAWVHAAPWEMMFVLFAALAAISFFGLWRAMPETATRLGEKLSLRELGRDYKAVLKNLRFVSGALAIGFVSLPLLAWIAQSPVIIISGEQMSTYEYGLLQVPIFGALILGNLVLAKLTARRSVRSLIIMGGWPIMAGLALAAFATLLSSHAYLWMTAGLSIYAFGIGIANAGLVRLTLFASDISKGTVSAAMGMLQMTIFTVGIEVSKHAWIGGGNGLFNLFNLANGLLWLGLMVIFLKDKTVGASREG

SEQ ID NO: 49 - protein, 411 aa, Klebsiella oxytoca
MQNYSLSGKRLGRQALLFPLCLVLYEFSTYIGNDMIQPGMLSVVAEFGVGNEWVPTSMTAYLAGGMFLQWLLGPLSDRIGRRPVMLTGVVWFIVTCLATLLAQTIEQFTLLRFLQGISLCFIGAVGYAAIQESFEEAVCIKITALMANVALIAPLLGPLVGAAWVHLLPWEMMFVLFAVLAAIAFFGLQKAMPETATRMGEKLSVKELGRDYREVLKNLRFVAGALATGFVSLPLLAWIAQSPVIIISGEQATSYEYGLLQVPIFGALIAGNLVLARQTSRKTVRSLIILGGWPIMIGLLISAVATVASTHAYLWMTAGLSVYAFGIGLANAGLVRLTLFASEMSKGTVSAAMGMLQMLIFTVGIEVSKHAYESGGSGLFSLLNLLSGVLWLAMTVYFLKDKRVGNAHEPQ

SEQ ID NO: 50 - protein, 410 aa, Citrobacter koseri
MQNLSQTGVRLGRQALLFPLCLVLYEFSTYIGNDMIQPGMLAVVEQYQAGIDWVPTSMTAYLAGGMFLQWLLGPLSDRIGRRPVMLAGVIWFIVTCLATLLAQNIEQFTLLRFLQGVSLCFIGAVGYAAIQESFEEAVCIKITALMANVALIAPLLGPLVGAAWVHVLPWEGMFVLFAVLAAIAFVGLQRAMPETATRLGEKLSLKELGRDYTLVLKNVRFVAGALALGFVSLPLLAWIAQSPIIIISGEQLSSYEYGLLQVPVFGALIAGNLVLARLTSRRTVRALIIMGGWPIVAGLLIAAAATVVSSHAYLWMTAGLSVYAFGIGVANAGLVRLTLFASDMSKGTVSAAMGMLQMLIFTVGIEVSKHAYLSGGNGLFSLFNLANGILWLLLMVIFLKDKRVGDSREG

SEQ ID NO: 51 - protein, 410 aa, Escherichia marmotae
MQNKLATGARLGRQALLFPLCLVLYEFSTYIGNDMIQPGMLAVVEQYQAGIDWVPTSMTAYLAGGMFLQWLLGPLSDRIGRRPVMLAGVVWFIITCLAILLAQNIEQFTLLRFLQGISLCFIGAVGYAAIQESFEEAVCIKITALMANVALIAPLLGPLVGAAWIHVLPWEGMFVLFAALAAISFFGLQRAMPETATRIGEKLSLKELGRDYKLVLKNSRFVAGALALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLANGVLWLLLMFIFLKDKQTGNSNDG

SEQ ID NO: 52 - protein, 408 aa, Shigella flexneri
MQNKLASGARLGRQALLFPLCLVLYEFSTYIGNDMIQPGMLAVVEQYQAGIDWVPTSMTAYLAGGMFLQWLLGPLSDRIGRRPVMLAGVVWFIVTCLAILLAQNIEQFTLLRFLQGISFCFIGAVGYAAIQESFEEAVCIKITALMANVALIAPLLGPLVGAAWIHVLPWEGMFVLFAALAAISFFGLQRAMPETAMRIGEKLSLKELGRDYKLVLKNGRFVAGALGFVSLPLLAWIAQSPIIIITGEQLSSYEYGLLQVPIFGALIAGNLLLARLTSRRTVRSLIIMGGWPIMIGLLVAAAATVISSHAYLWMTAGLSIYAFGIGLANAGLVRLTLFASDMSKGTVSAAMGMLQMLIFTVGIEISKHAWLNGGNGLFNLFNLVNGILWLSLMVIFLKDKQMGNSHGG

SEQ ID NO: 53 - protein, 410 aa, Salmonella enterica subsp. salamae
MHNRLQSGGRLGRQALLFPLCLVLYEFSTYIGNDMIQPGMLAVVAQYQASLDWVPTSMTAYLAGGMFLQWLLGPLSDRIGRRPVMLAGVVWFIVTCLATLLAKNIEQFTFLRFLQGISLCFIGAVGYAAIQESFEEAVCIKITALMANVALIAPLLGPLVGAAWVHVLPWEGMFILFAVLAAIAFFGLQRAMPETATRRGETLSFKVLGRDYRLVIKNRRFVAGALALGFVSLPLLAWIAQSPIIIISGEQLSSYEYGLLQVPVFGALIAGNLVLARLTSRRTVRSLIVMGGWPIVAGLIIAAAATVVSSHAYLWMTAGLSVYAFGIGLANAGLVRLTLFSSDMSKGTVSAAMGMLQMLIFTVGIEVSKHAWLSGGNGLFSLFNLANGILWLLLMLVFLKDKRTGDSQTG

SEQ ID NO: 54 - protein, 410 aa, Citrobacter youngae ATCC 29220
MQNRLSSGARLGRQALLFPLCLVLYEFSTYIGNDMIQPGMLAVVEQYNAGLDWVPTSMTAYLAGGMFLQWLLGPLSDRIGRRPVMLTGVLWFIVTCLATLLAQNIEQFTFLRFLQGISLCFIGAVGYAAIQESFEEAVCIKITALMANVALIAPLLGPLVGAAWVHILPWEGMFILFAALASISFFGLQRAMPETATRLGEKLSIKELGKDYKLVLKNGRFVAGALALGFVSLPLLAWIAQSPIIIISGEHLSSYEYGLLQVPIFGALIAGNLALARLTSRKTVRSLIIMGGWPIAVGLVIAAAATVVSSHAYLWMTAGLSVYAFGIGLANAGLVRLTLFASEMSKGTVSAAMGMLQMLIFTVGIELSKHAYLLGGNGLFSLFNLASGVLWLILMVIFLKDKRVGNSRKI

SEQ ID NO: 55 - protein, 410 aa, Citrobacter freundii
MQNRLSSGARLGRQALLFPLCLVLYEFSTYIGNDMIQPGMLAVVEQYQAGLDWVPTSMTAYLAGGMFLQWLLGPLSDRVGRRPVMLTGVVWFIVTCLATLFAQNIEQFTFLRFLQGISLCFIGAVGYAAIQESFEEAVCIKITALMANVALIAPLLGPLVGAAWVHVLPWEGMFVLFAALAAIAFFGLQRAMPETATRLGEKLSIKELGKDYKLVLKNVRFVAGALALGFVSLPLLAWIAQSPIIIISGEHLSSYEYGLLQVPIFGALIAGNLVLARLTSRRTVRSLIIMGGWPISVGLIIAAAATVVSSHAYLWMTAGLSLYAFGIGVANAGLVRLTLFASEMSKGTVSAAMGMLQMLIFTVGIELSKHAYLLGGNGLFSLFNLASGVLWLILMVIFLKDKRVGNSREG

SEQ ID NO: 56 - protein, 409 aa, Enterobacter kobei
MQNHSLPGRRLGRQALLFPLCLVLYEFSTYIANDMIQPGMLAVVEQYNAGIEWVPTSMTAYLAGGMFLQWLLGPLSDRIGRRPVMLTGVVWFIVTCLATLLAQDIEQFTLLRFLQGVSLCFIGAVGYAAIQESFDEATCIKITALMANVALIAPLLGPLVGAAWVHAAPWEGMFVLFAVLAAIAFFGLHRAMPETATRLGEPLSLNALGRDYKAVLKNGRFVAGALATGFVSLPLLAWIAQSPIIIISAEGMSSYEYGLLQVPIFGALIIGNLVLARLTSRRTVRSLIIMGGGPIVAGLLVAAVATVASSHAYLWMTAGLSIYAFGIGLANAGLVRLTLFASDMSKGTVSAAMGMLQMAIFTVGIEVSKHAFLAGGNGLFSLFNLANGLIWLALMVVFLKDKTVGNALS

SEQ ID NO: 57 - protein, 411 aa, Enterobacter sp.
MLNRSSSGTRLGRQALLFPLCLVLYEFSTYIGNDMIQPGMLAVVAQYNAGIEWVPTSMTAYLAGGMFLQWLLGPLSDRIGRRPVMLTGVAWFIVTCLATLLAQTIEQFMVLRFLQGVSLCFIGAVGYAAIQESFEEAVCIKITALMANVALIAPLLGPLVGAAWVHVAPWEGMFVLFAVLAAISFYGLHRAMPETATRIGEKLSLQELGRDYKEVLKNGRFVAGALAIGFVCLPLLAWIAQSPVIIISGENLSSYEYGLLQVPIFGALIVGNIVLARLTSRRTVRSLIIMGGWPIVIGLVVAAVATVVSSHAYLWMTAGLSIYAFGIGLANAGLVRLTLFASEVSKGTVSAAMGMLQMLIFTVGIEVSKHAFSSGGNGLFSLFNLVNGLLWLALMFVFLKDKRVGSSLQPG

SEQ ID NO: 58 - protein, 409 aa, Lelliottia sp. WB101
MLNRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLAILLAQTIEQFTLLRFLQGVSLCFIGAVGYAAIQESFEEAVCIKITALMANVALIAPLLGPLVGAAWVHVAPWEGMFVLFAALAAISFFGLHRAMPETATRLGEKLSLKELGRDYKEVLRNGRFVAGALATGFVSLPLLAWIAQSPVIIISGEKLSSYEYGLLQVPVFGALIIGNLVLARLTSRRSVRSLIIMGGWPIVAGLVVAAVATVASSHAYLWMTAGLSIYAFGIGLANAGLVRLTLFASEMSKGTVSAAMGMLQMLIFTVGIEVSKHAYSFGGNGLFSLFNLANGVLWLGLMVMFLKDKRVGSALQ

SEQ ID NO: 59 - protein, 410 aa, Enterobacter ludwigii EcWSU1
MLNRSSSGSRLGRQALLFPLCLVLYEFSTYIGNDMIQPGMLAVVEQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFVLFAVLAAVAFFGLHRAMPETATRLGEKLSLKELGRDYKEVLKNGRFVAGALATGFVSLPLLAWIAQSPVIIISGEQLSSYEYGLLQVPIFGALIIGNLVLARLTSRRTVRALIIMGGWPIAAGLVLAAVATVVSSHAYLWMTAGLSVYAFGIGVANAGLVRLTLFASEMSKGTVSAAMGMLQMLIFTVGIEVSKHAYAFGGNGLFSLFNLANGVLWVGLIVVFLKDKRVGNALQP

SEQ ID NO: 60 - protein, 410 aa, Enterobacter soli LF7a
MLNRSSSGNRLGRQALLFPLCLVLYEFSTYIGNDMIQPGMLAVVEQYNAGIEWVPTSMTAYLAGGMFLQWLLGPLSDRIGRRPVMLTGVVWFIVTCLATLLAQNIEQFTLLRFLQGVSLCFIGAVGYAAIQESFEEAVCIKITALMANVALIAPLLGPLVGAAWVHVAPWEGMFILFAALAAISFFGLHRAMPETATRLGEKLSLKDLGRDYKEVLKNIRFVAGALATGFVSLPLLAWIAQSPVIIISGEQLSSYEYGLLQVPIFGALIIGNLVLARLTSRRTVRSLIIMGGWPIAAGLIVAAVATVASSHAYLWMTAGLSLYAFGIGLANAGLVRLTLFASDMSKGTVSAAMGMLQMLIFTVGIEVSKHAYALGGNGLFSLFNLANGVLWVGLMVVFLKDKRVGNALQP

SEQ ID NO: 61 - protein, 405 aa, Bifidobacterium longum subsp. infantis (strain Bi-26)
MALDVGKALKSKTLVVGVLSMSLLMSASNAVSGTIPAMKEAFSDYSAANVELLTTVPTIGSMVGTALTGLFANAIGRKKIAMAGFLISAVTGVIPAFFPYYWPILISRMFFGFGSALFVTLSVSYITDLYDGDMQRKLLGWRQAVGNLGDVVLLFVASLLITINWQSTYLIFFLLFVPMVLVGMFIPKEFDNFSIRSALVDDEGHVVDKSASQKQTTNWQVLWLAFIFLMVSMLYNVMSIKLASYVVDEGIGSASLATLIFSFLVVATILSGVLFDKVAKVTKRLTVTISEVVIGICFIATALTKNVPLMFALVLIAGFAWGIINPALTARFVDYSPAHSMNLTTSIVIIGINIGCLISPYFFALTASVFGNSSAGFAIIVGGALYLVMVVIELITLKVDKKLTV

SEQ ID NO: 62 - protein, 392 aa, Cedecea neteri
MLWFLTRARRFNPVYAAFMAVSFMIGVAGALQAPTLSLFLTREVEVRPFWVGLFYTVNAIAGIGVSLLLAKRSDSQGDRRKLIMVCCVMAVANCVLFAFNRHYLTLITLGVMFASIANTAMPQIFALAREYADRSAREVVMFSSIMRAQLSLAWVIGPPLSFMLALKYGFTTMFLIAAGIFVISLALIIFALPSVPRVEQPAEVAITQVSGWKDSNVRMLFIASMLMWTCNTMYIIDMPLWISQDLGLPDELAGLLMGTAAGIEIPAMILAGYYVKRFGKRNMMVAAVAAGILFYVGLILFHSKTALVVLQLFNAVFIGIIAGIGMLWFQDLMPGRPGSATTLFTNSISTGVILAGILQGALAEGFGHYSVYWLMAALAVIALFLTSRVKNV

SEQ ID NO: 63 - protein, 623 aa, Escherichia coli K12 MG1655
MPHSDELDAGNVLAVENLNIAFMQDQQKIAAVRNLSFSLQRGETLAIVGESGSGKSVTALALMRLLEQAGGLVQCDKMLLQRRSREVIELSEQNAAQMRHVRGADMAMIFQEPMTSLNPVFTVGEQIAESIRLHQNASREEAMVEAKRMLDQVRIPEAQTILSRYPHQLSGGMRQRVMIAMALSCRPAVLIADEPTTALDVTIQAQILQLIKVLQKEMSMGVIFITHDMGVVAEIADRVLVMYQGEAVETGTVEQIFHAPQHPYTRALLAAVPQLGAMKGLDYPRRFPLISLEHPAKQAPPIEQKTVVDGEPVLRVRNLVTRFPLRSGLLNRVTREVHAVEKVSFDLWPGETLSLVGESGSGKSTTGRALLRLVESQGGEIIFNGQRIDTLSPGKLQALRRDIQFIFQDPYASLDPRQTIGDSIIEPLRVHGLLPGKDAAARVAWLLERVGLLPEHAWRYPHEFSGGQRQRICIARALALNPKVIIADEAVSALDVSIRGQIINLLLDLQRDFGIAYLFISHDMAVVERISHRVAVMYLGQIVEIGPRRAVFENPQHPYTRKLLAAVPVAEPSRQRPQRVLLSDDLPSNIHLRGEEVAAVSLQCVGPGHYVAQPQSEYAFMRR

SEQ ID NO: 64 - protein, 464 aa, E. coli K12 MG1655
MPDAKKQGRSNKAMTFFVCFLAALAGLLFGLDIGVIAGALPFIADEFQITSHTQEWVVSSMMFGAAVGAVGSGWLSFKLGRKKSLMIGAILFVAGSLFSAAAPNVEVLILSRVLLGLAVGVASYTAPLYLSEIAPEKIRGSMISMYQLMITIGILGAYLSDTAFSYTGAWRWMLGVIIIPAILLLIGVFFLPDSPRWFAAKRRFVDAERVLLRLRDTSAEAKRELDEIRESLQVKQSGWALFKENSNFRRAVFLGVLLQVMQQFTGMNVIMYYAPKIFELAGYTNTTEQMWGTVIVGLTNVLATFIAIGLVDRWGRKPTLTLGFLVMAAGMGVLGTMMHIGIHSPSAQYFAIAMLLMFIVGFAMSAGPLIWVLCSEIQPLKGRDFGITCSTATNWIANMIVGATFLTMLNTLGNANTFWVYAALNVLFILLTLWLVPETKHVSLEHIERNLMKGRKLREIGAHD

SEQ ID NO: 65 - protein, 396 aa, E. coli K12 MG1655
MTTNTVSRKVAWLRVVTLAVAAFIFNTTEFVPVGLLSDIAQSFHMQTAQVGIMLTIYAWVVALMSLPFMLMTSQVERRKLLICLFVVFIASHVLSFLSWSFTVLVISRIGVAFAHAIFWSITASLAIRMAPAGKRAQALSLIATGTALAMVLGLPLGRIVGQYFGWRMTFFAIGIGALITLLCLIKLLPLLPSEHSGSLKSLPLLFRRPALMSIYLLTVVVVTAHYTAYSYIEPFVQNIAGFSANFATALLLLLGGAGIIGSVIFGKLGNQYASALVSTAIALLLVCLALLLPAANSEIHLGVLSIFWGIAMMIIGLGMQVKVLALAPDATDVAMALFSGIFNIGIGAGALVGNQVSLHWSMSMIGYVGAVPAFAALIWSIIIFRRWPVTLEEQTQ

SEQ ID NO: 66 - protein, 416 aa, E. coli K12 MG1655
MSLAKASLWTAASTLVKIGAGLLVGKLLAVSFGPAGLGLAANFRQLITVLGVLAGAGIFNGVTKYVAQYHDNPQQLRRVVGTSSAMVLGFSTLMALVFVLAAAPISQGLFGNTDYQGLVRLVALVQMGIAWGNLLLALMKGFRDAAGNALSLIVGSLIGVLAYYVSYRLGGYEGALLGLALIPALVVIPAAIMLIKRGVIPLSYLKPSWDNGLAGQLSKFTLMALITSVTLPVAYIMMRKLLAAQYSWDEVGIWQGVSSISDAYLQFITASFSVYLLPTLSRLTEKRDITREVVKSLKFVLPAVAAASFTVWLLRDFAIWLLLSNKFTAMRDLFAWQLVGDVLKVGAYVFGYLVIAKASLRFYILAEVSQFTLLMVFAHWLIPAHGALGAAQAYMATYIVYFSLCCGVFLLWRRRA

SEQ ID NO: 67 - protein, 578 aa, Helicobacter pylori strain ATCC 700392 / 26695
MAKKKHKISTLKYFLRSLKQIYMLITFKEKMVFFLLVLMAVFSSFVEVMSLTLLMPFITLASDPNRALDDKDWKMVYDFFHFSSPVRLMYFFSFCLVGIYLFRMFYGVSFTYLKGRFSNKKAYQIKQQLFLQHIKSNYLSHLNHNLDSLRDIINNKAEGMFMSFNAFLSLLTEITVIVFFYSTLILTNWKITLVFTMILALQIFLIVKKVTVLIKKKGEMAAKSKAQTLKVFSKFFSNFKITKLKDNHEEAHKLFGENSRKAHDTEIIYATLQVVPRYSIETVGFSLLILAVAYILFKYGEAKMVLPTISMYALALYRILPSVTGMINYYNEIAYNQLATNMVFKSLSKTIVEEDLVPLDFNEKITLQNISFAYKSKHPVLKDFNLTIQRGQKVALIGHSGCGKSTLADIIMGLTYPKSGEIFIDNTLLTNKNRRSWRKKIGYIPQNIYLFDGTVGDNIAFGSAIDEKRLIKVCKMAHIYDFLCEHEGLKTQVGEGGAKLSGGQKQRIGIARALYDNPEILVLDEATSALDNETESKIMDEIYQIAKNKTLIVIAHRLSTIERCEVIIDMSQHKDNLG

SEQ ID NO: 68 - protein, 407 aa, B. longum subsp. Infantis (strain ATCC 15697)
MSKIINYSEVLESKRLMVGVLSVSFLLSAGNAISGTIPAMEEAFSNISKANIETLTTIPTAGIMLGTVLSGVFSNYLGKKKSVLAGLIIALVGGVIPAFLPQYWPIFISRFLFGVGMGIFNPLSVSYITDLYVGDRQRSLLGYRNAVSNLGDTIMLFVAGILITFGWNITYLVFFALLIPIVLIILFVPKEFDNFDIRNSALDENGQISDSVSDVKPSTNLKVIEVGVVFMVITMLYNAIPLKFASYIVTEHIGTASTATWIFSFLVLAGIFSGVLFEKISKVFKRLTVFVFEIVIGVAYITIAFTYNIPLLTALVLISGFGWGIINPALTARLVDVSPINSMNLSTSIIVIFISVGSLISPYFFAMFAGLFGNDSAAFAIVVGGALYVVMAVLDFIKIKKNKELSI

SEQ ID NO: 69 - protein, 582 aa, E. coli K12 MG1655
MHNDKDLSTWQTFRRLWPTIAPFKAGLIVAGVALILNAASDTFMLSLLKPLLDDGFGKTDRSVLVWMPLVVIGLMILRGITSYVSSYCISWVSGKVVMTMRRRLFGHMMGMPVSFFDKQSTGTLLSRITYDSEQVASSSSGALITVVREGASIIGLFIMMFYYSWQLSIILIVLAPIVSIAIRVVSKRFRNISKNMQNTMGQVTTSAEQMLKGHKEVLIFGGQEVETKRFDKVSNRMRLQGMKMVSASSISDPIIQLIASLALAFVLYAASFPSVMDSLTAGTITVVFSSMIALMRPLKSLTNVNAQFQRGMAACQTLFTILDSEQEKDEGKRVIERATGDVEFRNVTFTYPGRDVPALRNINLKIPAGKTVALVGRSGSGKSTIASLITRFYDIDEGEILMDGHDLREYTLASLRNQVALVSQNVHLFNDTVANNIAYARTEQYSREQIEEAARMAYAMDFINKMDNGLDTVIGENGVLLSGGQRQRIAIARALLRDSPILILDEATSALDTESERAIQAALDELQKNRTSLVIAHRLSTIEKADEIVVVEDGVIVERGTHNDLLEHRGVYAQLHKMQFGQ

SEQ ID NO: 70 - protein, 525 aa, Neurospora crassa OR74A
MGIFNKKPVAQAVDLNQIQEEAPQFERVDWKKDPGLRKLYFYAFILCIASATTGYDGMFFNSVQNFETWIKYFGDPRGSELGLLGALYQIGSIGSIPFVPLLTDNFGRKTPIIIGCVIMIVGAVLQATAKNLDTFMGGRTMLGFGNSLAQIASPMLLTELAHPQHRARLTTIYNCLWNVGALVVSWLAFGTNYINNDWSWRIPALLQAFPSIIQLLGIWWVPESPRFLIAKDKHDEALHILAKYHANGDPNHPTVQFEFREIKETIRLEMESTKNSSYLDFFKSRGNRYRLAILLSLGFFSQWSGNAIISNYSSKLYETAGVTDSTAKLGLSAGQTGLALIVSVTMALLVDKLGRRLAFLASTGGMCGTFVIWTLTAGLYGEHRLKGADKAMIFFIWVFGIFYSLAWSGLLVGYAIEILPYRLRGKGLMVMNMSVQCALTLNTYANPVAFDYFGPDHSWKLYLIYTCWIAAEFVFVFFMYVETKGPTLEELAKVIDGDEADVAHIDIHQVEKEVEIHEHEGKSVA

SEQ ID NO: 71 - protein, 529 aa, Aspergillus oryzae RIB40
MIAVGKQREEDVSDPVLANLLAEDRTPWYKKPNLRRLYLILFPACMGIEITSGFDSQIINTVQIVYTWNKYFGRLTGDTVDGMPEYEVEPNLKGFLGAAYSLGAILSLPFVPWVNQRFGRRWTVMFGSCISLVGALLQGFSNGVGMYIVARMLLGFGIPYCIVAGSCLIGELGYPKERPILTSLFNSSYFIGQIVAAAVGLGTVTIASNWAWRIPSLLQLAPAMVQVVFVFFLPESPRYLISKDRHEEAFGILAKYHAEGDRNSVIVRAEIAQIERTIKLELEEAKQSWWDMFRTAGMRRRLLISAFLGLFTQWSGNTLISYYLSDLLDMVGITDSVTKSKINIGIACWGLVSGTALALTAPLFKRRTMYLTCATSLLCVYIGWTISMERFMTTEVRAAAILTIFFIFAYSPAYNLGYNALTYTYLIEIFPYFGRSRGLSWFQFYGRGSAFFATYVNPVGLDRISWRWLLVYCCWLAFELVFIYFLFPETSGRTLEELSFMFEGKEKANEVAAAVHKQIEVDGKTEGQA

SEQ ID NO: 72 - protein, 429 aa, Chitinophaga sp. CF118
MSFNVKNRIAELFSKRIFRDSFYYTIGAFLVKGINFFLVPVYTSYLSPSEYGILDLYLTFTNILTIFCSLGLSQLIFVEFYKIKGEERKRFFSVIFFGYALFGLIICAIGGVIFHFASEYFTKVALASMLSVILSSAIAYITFYQTNFFNYLRLAGRSKLFVYFSIITGVVNAGLNILFVVKGNMGYNGILLGNIIVLFASVVFGFFVIKDKIDFKFNIDLKVFSGYTKIGLAFILSSLCQWLINGADRWIVLNALNTTELGIYALAFKFSSFVDPLIITPITYAYLPYIFKKYAENDYSEKLMKIAGLVFLIFLPIALLVPYLLPMVISNKDYYMAVQLIPFLIIAYYFYFISQLAGNVLVYLKKIRCLVQNIIISGISNIILNYILVRKMGIIGCAYAYLISNIIWAILNIYYRNMYLSSLKNEKSN

SEQ ID NO: 73 - protein, 470 aa, Eubacterium sp. CAG:581
MNRNKKLGINVVLMSISSFGSKLLTFFLVPLYTSYLTTAEYGVSDLITTTTSLLAPIFTATIGEAVLRYALEKDIDKYQVFRIGLFIHMVGFIALMCFSPLLLMIKLLKPYYVFFILYYFSFTFYSFFSLFCRGINKVVAFTISGIIQTLVMVGTNIVSLIFLDMGIYGYLLSFIVSNFAGMILLVTLGKMHVYFKIGKIDKKLMKKMLFYSLPMITNSISWWISNSSDKYILTFLCGATINGIYSVAYKIPTILSVCYGVFMSAWRLSAVDDFGSEETDRFYSQILTKMTKALIIVGAGIVLFNKILAKFLYAKDFFSAREFVPVLVIAFLLHGLGEFYGSVYTSAKCTKMLFYSSLAGAVCNIVLNFALIPEFQAMGAAIATMISYGVILAIRAIHSRTIMKMTIPVTNITISSVIFITMAIIQTIDFQGSFIISAILFCIIAFFNRDMVVEIINTVLKKKPKKTAKQ

SEQ ID NO: 74 - protein, 410 aa, Lactococcus raffinolactis KACC 13441
MFLAVWEGILRFGLNENNREKLTSLISTTVFFGLGATIFWAIILPFPYLRIFNTLPYVHLFIISLLLQPILSTLQIAVRGIKESKNYVISGIISTFVNVGLLIFLVVFLRLGLLGLLLSFLIGNLVNVLYLLFGSSLLKYISFKAIDKNLLKKLLKFSWPLIFNLVFVWFLGGYVMFYLGSFVGSDETGFFSFANKFASIVAMFGSVLGMATIEDTIITSENDNFIESFAKKNTEMFKMFLNVGILLLPVIGMYYFTLGNSAYQNTLIIVPFLLMASIFQAMATNVGNIMIVHQKTKAIAVASLLVGIFNAIFCFVGYHFLGLTGVCIAYLFASFLLFLFRYKMGQKIQNYNLKWRFIVVLAVIFIMLGLLIQFENVIINSILVLVTAVFEIFIYRHIILKILNRIIGRA

SEQ ID NO: 75 - protein, 434 aa, Prevotella ruminicola (AR32)
MTESRMKRSLTGTFWSMTERFAKMGIQIVCTFIIAQFVAPSEFGLVSMMSIFLAFSTILIDSGFSQALIHEQNVTPQDESSIFWFNIGLGCAVYGIFWLIAPLIANFYNEPQLTLLIRVAFLALIFQSLIVVQQGLLFKSVDFKAVSKISFWAVLLSGIAGIVVSYIRKDVWGLIVQNLLFALLQTVLYWVYSRWRPSLIFKMQCVRKYLRFSMNLLGSNMLAAITDNLANLFVGKAYNATILGHYTMANKIPYLTSGTVCYGIKRVSYSIMSTFQNDNEQLARYSQRVVGTAFWILSPVMILMLVLAEPFISWLFPEEWAPAAIYLRYFCVIGLVYCFADVNQDILLVKGRTDILFRLDIVRRTVLVALLIIGIQYSMETFLLLLVIYNILNAIIVSYIAGRLIDCSLWRQIRLVGSTPLYFLTNINQRREKR

SEQ ID NO: 76 - protein, 616 aa, Candidatus Planktophila sulfonica
MIEFLSKPKFSILTKSPIYRASRVLSVKDRRKISLVIVVQVCLGLFDLAGVAVFGVMGSLAVSGVQSQSPNSRVGQVLDAIGIANLPFQKQAAILAILATCFLMARTFLTVVVTKRTMLFLSRRGAAISSDLVARLLSKSLLFIQERTTQQTLFALTQGVSTLTIGVLATVVTMISDTSLLILLTAGLFVVDPAIAISTLVVFGSIILFMYKNLHNKAKTLGFAESTLHIKSNEKVLEVLNSYRESVVRNRRNYYSEQIGMLRFDLAKTQAQLSFMPYIGKYVLETSVVLGSLLIAAIQFALHDAVTAVATLSIFLVAGTRISPAALRLQQALIQIKSSLGGSEPTLDLIDALRDTLPATSVNQELDLTHEQFVPRIQIQDISFTYPNSDRQALSGVNLDIQSGSVVALVGPSGGGKTTLVDVILGIIEPNSGKVLISNLTPIDAIQISPGAISYVPQDVSIIDGTVRENIAMGFPVEVATDELVHNAIELAGLKEFVATLPNGLDTYVGEKGARISGGQRQRLGIARALFTNPKLLVLDEATSALDGETESRITDSILSLKGKVTVLMVAHRLSTVRNADQVVYMSEGSIKAVGTFESVRNTVPDFDTQAQLMGL

SEQ ID NO: 77 - protein, 602 aa, Butyrivibrio hungatei XBD2006
MSNTDNKKVSLFSKLRYIFDRKQKGQLVILAVLILFGGIFETFSISMMIPVMAGIINQDKLQSAIEDNKVLRIIAESLNLGTGSELAAKLTVCLIVLFLVKNAYQIFLIYRQNTFITRARNDMISRVMREFLNRPYEDYLGADIPTVFRITDSDIPRTFTLMLSLLSLSTELVVSMGLGMVLIITDPILTVICVFVFVALTLFNTKVLKPKLNKTGKENQETQSRIAKWRLQAIYGLKDVKVLNRQDFFIRNYYESGKVGADLARDYSVLNNMPRTLIETVFVAVVLLYVLFFKLNSNGVQNAGDVDKLIIALGLVATRLMPAVNRINTYIAEIAYNQYSLDFVYNNLTDSMKMDKAMRAERAAIAGPELHLEKEIEIKDITFAYPDAETNIFTGANMVIPKGKSVGIIGPSGAGKSTVVDIILGLLHVQSGEILCDGSNIFSNYDSWLAQIGYIPQTIYMVDESIRENIAFGIDADKIDEDRIWEVMEEAQLAEFVKSLPEGLDTKIGDRGVRLSGGQRQRIGIARALYHNPEILVFDEATSALDNETEAAVMEAVNSFHGKKTMIIIAHRLNTIENCDIIYEVKDEKITQTTLEGRNVIH

SEQ ID NO: 78 - protein, 581 aa, Roseburia intestinalis CAG:13
MKDLVKSVLKVFDGKQKRKLVGMMFLILINSGVSLLGVSVLSPFITAVMNPEELLKNDIIRSVYDGFHMQNTNQLITLLSVLIIIVYIAKNAFIIFINNMLYCFSYYGKREMQDRMMKYYISRDYTFFLNHNSAELMRDINTDPEMFYAAVLNMLQLASELCVSLILVGYLLVKDALLTLGVAFAMVVMVFIFMKKLRRTLARFGDDRRKYNANILQCMQQAFGGIKEIKIANREAYFEGEFVKQNGLYTYVIKQNSFLSSIPKPIMEALCITGLMAAIIVRINATSTSPEQFVGTLAVFAAAAFALLPSANKMSEYLGSIIHNGVVIHKIGEEYAAIRDMEIQTEKEENYKKVTLDKEIKVEDMTFHYPDTEDAVLAHVNVTIPKNKSVAFIGPSGAGKTTMVDLILGVLKPQAGKITVDGMDIKESYRGWHDKIGYIPQTIYMLDDTIRNNIAFGKTEGIDDADIWEALKQAQLDEFVKSLDEGLDTMIGEAGVRLSGGQRQRIGIARALYRRPEVLVLDEATSALDTETEAAVMEAIDSLQGKMTMLIIAHRLSTIKNCDMVYQVEGGSVTRKEKESV

SEQ ID NO: 79 - protein, 608 aa, Pedobacter ginsengisoli
MKIYFRLLSFAKPIEKFAIPYVITTVLSVIFSVLNLTLLAPLFETLISDEPTKSSGLADNAASAFNITAQFKEFVNSSIITNGQEKTLTYICLIIVLSVLLSNIFRYFSQRSMEDLRVHTLLNLRKTVFENVMNLHVGYFNNERKGDIISKVSADVQVVQFTVTNTLQVVFKEPLTLIFYVLVLLSISIKLTLFSLLVIPISAFVISKIVKRIKHQAKEAHESFAKMIGFLDEALGGIKIIKAFNASERIKERFHNENVFYSNINRKMVRRQQLGSPLSEFLSIVMVSFIVWYGGRLIISHQPGALSTSQFIAYIAIFSQVMRPAKALTDSFSGIHTGIAAGERVLDLIDTKAEQINKPDAKEISSFNNSLVFENVSFSYESKEVLKNINLTIQKGKTIALVGPSGGGKSTLMDLIPRFHDPKSGSIKIDGLDYADLTVESIRSQLGMVNQESILFNDTIFNNIAFAKPDATEAEVIAAAKIANAHDFILQTENGYETYTGDRGNKLSGGQKQRLCIARAILANPPIMLLDEATSALDTESEKLVQDALNKLMENRTSLVIAHRLSTIHGADLIVVIDKGEIIETGTHQELLSHNGLYKKLIELQTFS

SEQ ID NO: 80 - protein, 576 aa, Verrucomicrobia bacterium CG1_02_43_26
MKKLFPYLVYLKDVKMAFILAILAGIFYGASTGLGIPVLIRYLYPKIFSNEAISVMTLTLICTLPVIVSAFRAGGNFLNSYYLSYCGQYILQQLRIKVFAKIQNLPIAFFHKRQPGDLISRVSNDTTILQQTIINASQEILKQPITLLGAVSYIVYMCFQQSDVVFLIIFILAIPLCIVPIRYIGIKMRNKARAMQEQTSELTHRLAQNLSAVKEIRSFCLEEYELSRYEAVCHVLRERFLKVVKYTSILSPSIEVIASFGVAMAFWYSYKSKIEPDVFISIVGALYLSYEPIKKLGRLNSEMQQGLASLERLENILEEPITIHDPAVPHPVGKLEGNIQFKNVSFAYEMAPVLRDVTVHLTAKKTYALVGPSGAGKTTFAHLIPRFYDLAEGNGSITIDGINIKDMRLKDLRQNIALVSQDPVLFNDTIYNNILVGKPSASHEELMTAAKRAFAHHFILELENGYDTLVGENGSMLSGGQKQRIALARAFLKDAPILILDEATSALDANSEQLIQQALEELFKGKTVIIIAHRFSTIKHADRIFVFKSGEIIEEGTHDLLCEGKGLYEELYAKQS

SEQ ID NO: 81 - protein, 572 aa, Actinobaculum suis DSM 20639
MGKSQESISFKEKFAYGMGDAATAFSAVSVASFSMFYFTDYVGVSATALGTVLIFTRVFDAITNIIMGYVVDQTRTKEGKARPWVKWAILPMFAALVAVFAIPTGMSSTATIVWITIVVNIYYLVYTISNIPYGTLGTLISRDPQVRSELTLLRMIGYHGVSILLSFITVGLVAYLGGGSNRGWIYTMVIYGAIMSIIFYFTYSCTRERVRSTSEMRRDAERAAAAESGAANSTPPNGGPEAGTGTAPAKSAGATGTAPAKPAPAKRLGIFASIGLLFRNKYWILIFLIMILTWILVNLFGGVNVYYARHILGDANYVGVLNAAFTGAQIVGFFLVSIPIRRFGRRRTIVFGLGMIIVGSLLVLTGPQNIYVLAVASIIRGLGFSPLMGTAYAMLADVIDYGEWKNGVRNEGISYSGGTFSTTVGSGLASSGIVWIMGAAGYISAKDAVQPASVLESIQFLFSWAPVIIAALLLVLFYFYDLDTFYPEIARDLEQGITLKDREKDAARQAHHVSGVEPAHPDHHQNNPESPAGSTQTSAQTGTQTSTKYRTEKGGQNRTNYSTESEAGNAVE

SEQ ID NO: 82 - protein, 453 aa, Ruminococcus gnavus
MEKKRYLKWYNKVGYGSGDLAGNVVYAFLSSFVMLYLTNTVGLNPGIIGTLIMVSKLFDGISDMFFGTMIDKTKSKLGKARPWMLYAYIGCAVTLVANFAIPDSLGTTAQYAWFFIAYTLLNAVFFTANNIAYASLVTFCTKNSRERVEMGSWRFIFAFSTSLLIQSVTVQFVRAAGGGAAAWRTVAVVYAIIGLIVNTISVFSIKELPEEELKAGKDHTEEKYSLVEAAKLLFSNKYYLMICATYICQQIYSAMLNMGIYYMIYILKNENLYSVFSWAINIPVIIAMCITPMLVEKMKGLYRMNLAGYILGTAGRVGVIFAGYMGSVPLMLAFTAVAALGMAPWQGDMGAVVASCSEYTYLTKHKHIDGTMYSCTSFGTKIGGGIGVALCGWLLDASGFVKEATIQPESCLNMLHVMYLWIPMVLSLIITFIMSRMNVEDANEKLRKQLEEC

SEQ ID NO: 83 - protein, 590 aa, E. coli K12 MG1655
MRLFAQLSWYFRREWRRYLGAVALLVIIAMLQLVPPKVVGIVVDGVTEQHFTTGQILMWIATMVLIAVVVYLLRYVWRVLLFGASYQLAVELREDYYRQLSRQHPEFYLRHRTGDLMARATNDVDRVVFAAGEGVLTLVDSLVMGCAVLIMMSTQISWQLTLFSLLPMPVMAIMIKRNGDALHERFKLAQAAFSSLNDRTQESLTSIRMIKAFGLEDRQSALFAADAEDTGKKNMRVARIDARFDPTIYIAIGMANLLAIGGGSWMVVQGSLTLGQLTSFMMYLGLMIWPMLALAWMFNIVERGSAAYSRIRAMLAEAPVVNDGSEPVPEGRGELDVNIHQFTYPQTDHPALENVNFALKPGQMLGICGPTGSGKSTLLSLIQRHFDVSEGDIRFHDIPLTKLQLDSWRSRLAVVSQTPFLFSDTVANNIALGCPNATQQEIEHVARLASVHDDILRLPQGYDTEVGERGVMLSGGQKQRISIARALLVNAEILILDDALSAVDGRTEHQILHNLRQWGQGRTVIISAHRLSALTEASEIIVMQHGHIAQRGNHDVLAQQSGWYRDMYRYQQLEAALDDAPENREEAVDA

SEQ ID NO: 84 - protein, 593 aa, E. coli K12 MG1655
MRSFSQLWPTLKRLLAYGSPWRKPLGIAVLMMWVAAAAEVSGPLLISYFIDNMVAKNNLPLKVVAGLAAAYVGLQLFAAGLHYAQSLLFNRAAVGVVQQLRTDVMDAALRQPLSEFDTQPVGQVISRVTNDTEVIRDLYVTVVATVLRSAALVGAMLVAMFSLDWRMALVAIMIFPVVLVVMVIYQRYSTPIVRRVRAYLADINDGFNEIINGMSVIQQFRQQARFGERMGEASRSHYMARMQTLRLDGFLLRPLLSLFSSLILCGLLMLFGFSASGTIEVGVLYAFISYLGRLNEPLIELTTQQAMLQQAVVAGERVFELMDGPRQQYGNDDRPLQSGTIEVDNVSFAYRDDNLVLKNINLSVPSRNFVALVGHTGSGKSTLASLLMGYYPLTEGEIRLDGRPLSSLSHSALRQGVAMVQQDPVVLADTFLANVTLGRDISEERVWQALETVQLAELARSMSDGIYTPLGEQGNNLSVGQKQLLALARVLVETPQILILDEATASIDSGTEQAIQHALAAVREHTTLVVIAHRLSTIVDADTILVLHRGQAVEQGTHQQLLAAQGRYWQMYQLQLAGEELAASVREEESLSA

SEQ ID NO: 85 - protein, 306 aa, E. coli K12 MG1655
MLNYVIKRLLGLIPTLFIVSVLVFLFVHMLPGDPARLIAGPEADAQVIELVRQQLGLDQPLYHQFWHYISNAVQGDFGLSMVSRRPVADEIASRFMPTLWLTITSMVWAVIFGMAAGIIAAVWRNRWPDRLSMTIAVSGISFPAFALGMLLIQVFSVELGWLPTVGADSWQHYILPSLTLGAAVAAVMARFTRASFVDVLSEDYMRTARAKGVSETWVVLKHGLRNAMIPVVTMMGLQFGFLLGGSIVVEKVFNWPGLGRLLVDSVEMRDYPVIQAEILLFSLEFILINLVVDVLYAAINPAIRYK

SEQ ID NO: 86 - protein, 303 aa, E. coli K12 MG1655
MRLFNWRRQAVLNAMPLVKPDQVRTPWHEFWRRFRRQHMAMTAALFVILLIVVAIFARWIAPYDAENYFDYDNLNNGPSLQHWFGVDSLGRDIFSRVLVGAQISLAAGVFAVFIGAAIGTLLGLLAGYYEGWWDRLIMRICDVLFAFPGILLAIAVVAVLGSGIANVIIAVAIFSIPAFARLVRGNTLVLKQQTFIESARSIGASDMTVLLRHILPGTVSSIVVFFTMRIGTSIISAASLSFLGLGAQPPTPEWGAMLNEARADMVIAPHVAVFPALAIFLTVLAFNLLGDGLRDALDPKIKG

SEQ ID NO: 87 - protein, 306 aa, E. coli K12 MG1655
MLKFILRRCLEAIPTLFILITISFFMMRLAPGSPFTGERTLPPEVMANIEAKYHLNDPIMTQYFSYLKQLAHGDFGPSFKYKDYSVNDLVASSFPVSAKLGAAAFFLAVILGVSAGVIAALKQNTKWDYTVMGLAMTGVVIPSFVVAPLLVMIFAIILHWLPGGGWNGGALKFMILPMVALSLAYIASIARITRGSMIEVLHSNFIRTARAKGLPMRRIILRHALKPALLPVLSYMGPAFVGIITGSMVIETIYGLPGIGQLFVNGALNRDYSLVLSLTILVGALTILFNAIVDVLYAVIDPKIRY

SEQ ID NO: 88 - protein, 302 aa, E. coli K12 MG1655
MMLSKKNSETLENFSEKLEVEGRSLWQDARRRFMHNRAAVASLIVLVLIALFVILAPMLSQFAYDDTDWAMMSSAPDMESGHYFGTDSSGRDLLVRVAIGGRISLMVGVAAALVAVVVGTLYGSLSGYLGGKVDSVMMRLLEILNSFPFMFFVILLVTFFGQNILLIFVAIGMVSWLDMARIVRGQTLSLKRKEFIEAAQVGGVSTSGIVIRHIVPNVLGVVVVYASLLVPSMILFESFLSFLGLGTQEPLSSWGALLSDGANSMEVSPWLLLFPAGFLVVTLFCFNFIGDGLRDALDPKDR

SEQ ID NO: 89 - protein, 337 aa, E. coli K12 MG1655
MSVIETATVPLAQQQADALLNVKDLRVTFSTPDGDVTAVNDLNFSLRAGETLGIVGESGSGKSQTAFALMGLLAANGRIGGSATFNGREILNLPEHELNKLRAEQISMIFQDPMTSLNPYMRVGEQLMEVLMLHKNMSKAEAFEESVRMLDAVKMPEARKRMKMYPHEFSGGMRQRVMIAMALLCRPKLLIADEPTTALDVTVQAQIMTLLNELKREFNTAIIMITHDLVVVAGICDKVLVMYAGRTMEYGNARDVFYQPVHPYSIGLLNAVPRLDAEGETMLTIPGNPPNLLRLPKGCPFQPRCPHAMEICSSAPPLEEFTPGRLRACFKPVEELL

SEQ ID NO: 90 - protein, 492 aa, E. coli K12 MG1655
MSLREKTISGAKWSAIATVIIIGLGLVQMTVLARIIDNHQFGLLTVSLVIIALADTLSDFGIANSIIQRKEISHLELTTLYWLNVGLGIVVCVAVFLLSDLIGDVLNNPDLAPLIKTLSLAFVVIPHGQQFRALMQKELEFNKIGMIETSAVLAGFTCTVVSAHFWPLAMTAILGYLVNSAVRTLLFGYFGRKIYRPGLHFSLASVAPNLRFGAWLTADSIINYLNTNLSTLVLARILGAGVAGGYNLAYNVAVVPPMKLNPIITRVLFPAFAKIQDDTEKLRVNFYKLLSVVGIINFPALLGLMVVSNNFVPLVFGEKWNSIIPVLQLLCVVGLLRSVGNPIGSLLMAKARVDISFKFNVFKTFLFIPAIVIGGQMAGAIGVTLGFLLVQIINTILSYFVMIKPVLGSSYRQYILSLWLPFYLSLPTLVVSYALGIVLKGQLALGMLLAVQIATGVLAFVVMIVLSRHPLVVEVKRQFCRSEKMKMLLRAG

SEQ ID NO: 91 - protein, 461 aa, E. coli K12 MG1655
MSHITTEDPATLRLPFKEKLSYGIGDLASNILLDIGTLYLLKFYTDVLGLPGTYGGIIFLISKFFTAFTDMGTGIMLDSRRKIGPKGKFRPFILYASFPVTLLAIANFVGTPFDVTGKTVMATILFMLYGLFFSMMNCSYGAMVPAITKNPNERASLAAWRQGGATLGLLLCTVGFVPVMNLIEGNQQLGYIFAATLFSLFGLLFMWICYSGVKERYVETQPANPAQKPGLLQSFRAIAGNRPLFILCIANLCTLGAFNVKLAIQVYYTQYVLNDPILLSYMGFFSMGCIFIGVFLMPASVRRFGKKKVYIGGLLIWVLGDLLNYFFGGGSVSFVAFSCLAFFGSAFVNSLNWALVSDTVEYGEWRTGVRSEGTVYTGFTFFRKVSQALAGFFPGWMLTQIGYVPNVAQADHTIEGLRQLIFIYPSALAVVTIVAMGCFYSLNEKMYVRIVEEIEARKRTA

SEQ ID NO: 92 - protein, 401 aa, Azospirillum brasiliense LMG 04375
MRRTPPRMSAAASDRLPLASLLALAMAAFITILTEALPAGLLPQMAQGLGVTEAWVGQTVTIYAAGSLAAAIPLTAATQGVRRRPLLLAAIAGFVVANTVTTLSGSFALTMVARFLAGVSAGLMWALLAGYAARMVPDHQKGRAIAVAMVGAPLALSLGVPTGTFLGNLVDWRVCFGLMSALALALMLWVRVVVPDFAGQAAGRRLSPGRVFTLAGVRPVLFVVLAFVLAHNILYTYIAPFLAAAGMEGRTDLALLLFGVASLLGIWIVGALIDGHLRALTLASTVLFGLSALVLGVAGDAPAVVLAAVAAWGLAFGGAGTLFQTALARTAGDAADVAQSMLVTAWNTAIAGGGLLGGVLLERLGVGAFAPAVLVLLAATLLAVWGARRHGFPDPADTAGA

SEQ ID NO: 93 - protein, 262 aa, Bradyrhizobium japonicum USDA 110
MDDGYASVMPANAYNWTAVWRRNYLAWRKVALASLLGNLADPITNLFGLGFGLGLIVGRVEGTSYIAFLAAGMVAISAMTSATFETLYAAFARMDVKRTWEGILFTQLTLGDIVLGELVWAASKSVLAGTAIGIVAATLGYASWTSVLCAIPTIALTGLVFASLAMVVISLAPTYDYFVFYQSLVLTPMVFLCGAVFPTSQMPDSFQHFAGLLPLAHSVDLIRPVMLERGADNAALHVGALCVYAVLPFFASIALFRRRLLR

SEQ ID NO: 94 - protein, 9 aa, Artificial sequence
  conserved domain
  UNSURE (2)..(2): Xaa can be any naturally occurring amino acid
  UNSURE (5)..(5): Xaa can be any naturally occurring amino acid
GXSGXGKST

SEQ ID NO: 95 - protein, 19 aa, Artificial sequence
  conserved domain
  UNSURE (5)..(5): Xaa can be any naturally occurring amino acid
  UNSURE (8)..(11): Xaa can be any naturally occurring amino acid
  UNSURE (14)..(17): Xaa can be any naturally occurring amino acid
SGGQXQRXXXXRAXXXXPK

SEQ ID NO: 96 - protein, 12 aa, Artificial sequence
  conserved domain
  VARIANT (1)..(1): Xaa can be Ala, Gly, Met or Ser
  UNSURE (2)..(2): Xaa can be any naturally occurring amino acid
  VARIANT (3)..(3): Xaa can be Phe, Leu, Met, Val or Tyr
  UNSURE (4)..(4): Xaa can be any naturally occurring amino acid
  VARIANT (5)..(5): Xaa can be Asp, Glu, Lys, Asn, Gln or Arg
  UNSURE (6)..(7): Xaa can be any naturally occurring amino acid
  VARIANT (8)..(8): Xaa can be Glu, Gly, Ser or Thr
  VARIANT (9)..(9): Xaa can be Pro, Arg, Thr, Val or Tyr
  VARIANT (10)..(10): Xaa can be Lys or Arg
  UNSURE (11)..(11): Xaa can be any naturally occurring amino acid
  VARIANT (11)..(11): Xaa can be Gly, Ile, Leu, Met or Val
  misc_feature (12)..(12): Xaa can be any naturally occurring amino acid
XXXXXXXXXXXX

SEQ ID NO: 97 - protein, 6 aa, Artificial sequence
  conserved domain
  VARIANT (1)..(1): Xaa can be Leu, Arg, Ser or Thr
  UNSURE (2)..(4): Xaa can be any naturally occurring amino acid
  VARIANT (5)..(5): Xaa can be Ala or Gly
  VARIANT (6)..(6): Xaa can be Ala, Phe, Ile, Leu or Val
XXXXXX

SEQ ID NO: 98 - protein, 8 aa, Artificial sequence
  conserved domain
  UNSURE (2)..(2): Xaa can be any naturally occurring amino acid
  UNSURE (4)..(4): Xaa can be any naturally occurring amino acid
  UNSURE (7)..(7): Xaa can be any naturally occurring amino acid
LXAXNRXY

SEQ ID NO: 99 - protein, 507 aa, Bifidobacterium longum subsp. Infantis (strain ATCC 15697)
MSNENTAVGDVRKKGGLGQRIAYACGNLGQAAFYNAMSTYFVTYVTSCLFVSYSKALAAQMIAVITGLIVVIRIAEIFIDPLLGNLVDNTTTKWGRFRPWQFIGGLVSSVLIMLIFSGMFGLVNVNTTLFIVLFVITFIVLDVFYSLRDISYWGMIPALSSDSHERSTYTALGTFTGSIGYNGITVIVIPIVSYFTWTFTGAKGQGQAGWTSFGFIVALLGLITAWAVAFGTKESTNALRAKAQKNGNPFEAFKALFQNDQLLWVALSYLLYAIANVITTGVMYYLFVFVLDEPAAFSVTGIIPLIAGFIMAPLYPILNRWIPRRYLFAGGMVSMIIGYTMLALFSSNLPVVIVALIFFYVPAQFIQMTAILSLTDSIEYGQLKNGKRNEAVTLSVRPMLDKIGGAMSNGVVGAVALAAGMTGHATAADMTASNITTFKTFAFYIPLVLIILSLVVFWFKVKIDEKMHAQIVDELEAKLASGEIVDDEAQTVEAVEAINEETPAAKN

SEQ ID NO: 100 - protein, 471 aa, Klebsiella pneumoniae MB369
MTDLPASVRWQLWIVAFGFFMQSLDTTIVNTALPSMAKSLGESPLHMHMIIVSYVLTVAVMLPASGWLADRVGVRNIFFTAIVLFTAGSLFCAQASTLDQLVMARVLQGIGGAMMVPVGRLTVMKIVPRDQYMAAMTFVTLPGQVGPLLGPALGGVLVEYASWHWIFLINIPVGIVGAIATLCLMPNYTMQTRRFDLAGFVLLAAGMATLTLALDGQKGLGISPAWLAGLVAVGLAALLLYLWHARGNARALFSLNLFRNRTFSLGLGGSFAGRIGSGMLPFMTPVFLQIGLGFSPFHAGLMMIPMVLGSMGMKRIVVQVVNRFGYRRVLVASTLGLAAVSLLFMFCALAGWYYALPLILFLQGMINASRFSSMNTLTLKDLPDDLASSGNSLLSMVMQLSMSIGVTIAGLLLGLYGQQHLSLDAAGTHQVFLYTYLSMAAIIALPALIFSRVPDDVGSNTVIRRRNRSGS

SEQ ID NO: 101 - protein, 401 aa, Azospirillum brasiliense LMG 04375; identical to SEQ ID NO: 92

SEQ ID NO: 102 - protein, 1049 aa, Escherichia coli str. K-12 substr. W3110 [sequence omitted]

SEQ ID NO: 103 - protein, 491 aa, Escherichia coli str. K-12 substr. W3110
MSSQYLRIFQQPRSAILLILGFASGLPLALTSGTLQAWMTVENIDLKTIGFFSLVGQAYVFKFLWSPLMDRYTPPFFGRRRGWLLATQILLLVAIAAMGFLEPGTQLRWMAALAVVIAFCSASQDIVFDAWKTDVLPAEERGAGAAISVLGYRLGMLVSGGLALWLADKWLGWQGMYWLMAALLIPCIIATLLAPEPTDTIPVPKTLEQAVVAPLRDFFGRNNAWLILLLIVLYKLGDAFAMSLTTTFLIRGVGFDAGEVGVVNKTLGLLATIVGALYGGILMQRLSLFRALLIFGILQGASNAGYWLLSITDKHLYSMGAAVFFENLCGGMGTSAFVALLMTLCNKSFSATQFALLSALSAVGRVYVGPVAGWFVEAHGWSTFYLFSVAAAVPGLILLLVCRQTLEYTRVNDNFISRTAYPAGYAFAMWTLAAGVSLLAVWLLLLTMDALDLTHFSFLPALLEVGVLVALSGVVLGGLLDYLALRKTHLT

SEQ ID NO: 104 - protein, 512 aa, Escherichia coli str. K-12 substr. W3110
MQQQKPLEGAQLVIMTIALSLATFMQVLDSTIANVAIPTIAGNLGSSLSQGTWVITSFGVANAISIPLTGWLAKRVGEVKLFLWSTIAFAIASWACGVSSSLNMLIFFRVIQGIVAGPLIPLSQSLLLNNYPPAKRSIALALWSMTVIVAPICGPILGGYISDNYHWGWIFFINVPIGVAVVLMTLQTLRGRETRTERRRIDAVGLALLVIGIGSLQIMLDRGKELDWFSSQEIIILTVVAVVAICFLIVWELTDDNPIVDLSLFKSRNFTIGCLCISLAYMLYFGAIVLLPQLLQEVYGYTATWAGLASAPVGIIPVILSPIIGRFAHKLDMRRLVTFSFIMYAVCFYWRAYTFEPGMDFGASAWPQFIQGFAVACFFMPLTTITLSGLPPERLAAASSLSNFTRTLAGSIGTSITTTMWTNRESMHHAQLTESVNPFNPNAQAMYSQLEGLGMTQQQASGWIAQQITNQGLIISANEIFWMSAGIFLVLLGLVWFAKPPFGAGGGGGGAH

SEQ ID NO: 105 - protein, 512 aa, Escherichia coli str. K-12 substr. W3110
MAITKSTPAPLTGGTLWCVTIALSLATFMQMLDSTISNVAIPTISGFLGASTDEGTWVITSFGVANAIAIPVTGRLAQRIGELRLFLLSVTFFSLSSLMCSLSTNLDVLIFFRVVQGLMAGPLIPLSQSLLLRNYPPEKRTFALALWSMTVIIAPICGPILGGYICDNFSWGWIFLINVPMGIIVLTLCLTLLKGRETETSPVKMNLPGLTLLVLGVGGLQIMLDKGRDLDWFNSSTIIILTVVSVISLISLVIWESTSENPILDLSLFKSRNFTIGIVSITCAYLFYSGAIVLMPQLLQETMGYNAIWAGLAYAPIGIMPLLISPLIGRYGNKIDMRLLVTFSFLMYAVCYYWRSVTFMPTIDFTGIILPQFFQGFAVACFFLPLTTISFSGLPDNKFANASSMSNFFRTLSGSVGTSLTMTLWGRRESLHHSQLTATIDQFNPVFNSSSQIMDKYYGSLSGVLNEINNEITQQSLSISANEIFRMAAIAFILLTVLVWFAKPPFTAKGVG

SEQ ID NO: 106 - protein, 416 aa, Escherichia coli
MNKQSWLLNLSLLKTHPAFRAVFLARFISIVSLGLLGVAVPVQIQMMTNSTWQVGLSVMLTGGAMFVGLMVGGVLADRYERKKVILLARGTCGIGFIGLCLNALLPESSLLAIYLLGLWDGFFASLGVTALLAATPALVGRENLMQAGAITMLTVRLGSVISPMMGGLLLATGGVAWNYGLAAAGTFITLLPLLSLPALPPPPQPREHPLKSLLAAFRFLLASPLVGGIALLGGLLTMASAVRVLYPALAINWQMSAAQIGFLYAAIPLGAAIGALTSGKLAHSVRPGLLMLLSTLGSFLAIGLFGLMPMWIPGVVCLALFGWLSAVSSLLQYTMLQTQTPEAMLGRINGLWTAQNVTGDAIGAALLGGLGAMMTPVTSASVSGFGLLIIGVLLLLVLVELRRFRQTPPQAAASDS

SEQ ID NO: 107 - protein, 475 aa, Escherichia coli str. K-12 substr. W3110
MSDKKKRSMAGLPWIAAMAFFMQALDATILNTALPAIAHSLNRSPLAMQSAIISYTLTVAMLIPVSGWLADRFGTRRIFTLAVSLFTLGSLACALSNSLPQLVVFRVIQGIGGAMMMPVARLALLRAYPRNELLPVLNFVAMPGLVGPILGPVLGGVLVTWATWHWIFLINIPIGIAGLLYARKHMPNFTTARRRFDITGFLLFGLSLVLFSSGIELFGEKIVASWIALTVIVTSIGLLLLYILHARRTPNPLISLDLFKTRTFSIGIVGNIATRLGTGCVPFLMPLMLQVGFGYQAFIAGCMMAPTALGSIIAKSMVTQVLRRLGYRHTLVGITVIIGLMIAQFSLQSPAMAIWMLILPLFILGMAMSTQFTAMNTITLADLTDDNASSGNSVLAVTQQLSISLGVAVSAAVLRVYEGMEGTTTVEQFHYTFITMGIITVASAAMFMLLKTTDGNNLIKRQRKSKPNRVPSESE

SEQ ID NO: 108 - protein, 453 aa, Escherichia coli str. K-12 substr. W3110
MEKENITIDPRSSFTPSSSADIPVPPDGLVQRSTRIKRIQTTAMLLLFFAAVINYLDRSSLSVANLTIREELGLSATEIGALLSVFSLAYGIAQLPCGPLLDRKGPRLMLGLGMFFWSLFQAMSGMVHNFTQFVLVRIGMGIGEAPMNPCGVKVINDWFNIKERGRPMGFFNAASTIGVAVSPPILAAMMLVMGWRGMFITIGVLGIFLAIGWYMLYRNREHVELTAVEQAYLNAGSVNARRDPLSFAEWRSLFRNRTMWGMMLGFSGINYTAWLYLAWLPGYLQTAYNLDLKSTGLMAAIPFLFGAAGMLVNGYVTDWLVKGGMAPIKSRKICIIAGMFCSAAFTLIVPQATTSMTAVLLIGMALFCIHFAGTSCWGLIHVAVASRMTASVGSIQNFASFICASFAPIITGFIVDTTHSFRLALIICGCVTAAGALAYIFLVRQPINDPRKD

SEQ ID NO: 109 - protein, 469 aa, Escherichia coli str. K-12 substr. W3110
MTTKLSYGFGAFGKDFAIGIVYMYLMYYYTDVVGLSVGLVGTLFLVARIWDAINDPIMGWIVNATRSRWGKFKPWILIGTLANSVILFLLFSAHLFEGTTQIVFVCVTYILWGMTYTIMDIPFWSLVPTITLDKREREQLVPYPRFFASLAGFVTAGVTLPFVNYVGGGDRGFGFQMFTLVLIAFFIVSTIITLRNVHEVFSSDNQPSAEGSHLTLKAIVALIYKNDQLSCLLGMALAYNVASNIITGFAIYYFSYVIGDADLFPYYLSYAGAANLVTLVFFPRLVKSLSRRILWAGASILPVLSCGVLLLMALMSYHNVVLIVIAGILLNVGTALFWVLQVIMVADIVDYGEYKLHVRCESIAYSVQTMVVKGGSAFAAFFIAVVLGMIGYVPNVEQSTQALLGMQFIMIALPTLFFMVTLILYFRFYRLNGDTLRRIQIHLLDKYRKVPPEPVHADIPVGAVSDVKA

SEQ ID NO: 110 - protein, 500 aa, Escherichia coli str. K-12 substr. W3110
MLKRKKVKPITLRDVTIIDDGKLRKAITAASLGNAMEWFDFGVYGFVAYALGKVFFPGADPSVQMVAALATFSVPFLIRPLGGLFFGMLGDKYGRQKILAITIVIMSISTFCIGLIPSYDTIGIWAPILLLICKMAQGFSVGGEYTGASIFVAEYSPDRKRGFMGSWLDFGSIAGFVLGAGVVVLISTIVGEANFLDWGWRIPFFIALPLGIIGLYLRHALEETPAFQQHVDKLEQGDREGLQDGPKVSFKEIATKYWRSLLTCIGLVIATNVTYYMLLTYMPSYLSHNLHYSEDHGVLIIIAIMIGMLFVQPVMGLLSDRFGRRPFVLLGSVALFVLAIPAFILINSNVIGLIFAGLLMLAVILNCFTGVMASTLPAMFPTHIRYSALAAAFNISVLVAGLTPTLAAWLVESSQNLMMPAYYLMVVAVVGLITGVTMKETANRPLKGATPAASDIQEAKEILVEHYDNIEQKIDDIDHEIADLQAKRTRLVQQHPRIDE

SEQ ID NO: 111 - protein, 438 aa, Escherichia coli str. K-12 substr. W3110
MDSTLISTRPDEGTLSLSRARRAALGSFAGAVVDWYDFLLYGITAALVFNREFFPQVSPAMGTLAAFATFGVGFLFRPLGGVIFGHFGDRLGRKRMLMLTVWMMGIATALIGILPSFSTIGWWAPILLVTLRAIQGFAVGGEWGGAALLSVESAPKNKKAFYSSGVQVGYGVGLLLSTGLVSLISMMTTDEQFLSWGWRIPFLFSIVLVLGALWVRNGMEESAEFEQQQHYQAAAKKRIPVIEALLRHPGAFLKIIALRLCELLTMYIVTAFALNYSTQNMGLPRELFLNIGLLVGGLSCLTIPCFAWLADRFGRRRVYITGTLIGTLSAFPFFMALEAQSIFWIVFFSIMLANIAHDMVVCVQQPMFTEMFGASYRYSGAGVGYQVASVVGGGFTPFIAAALITYFAGNWHSVAIYLLAGCLISAMTALLMKDSQRA

SEQ ID NO: 112 - protein, 457 aa, Escherichia coli str. K-12 substr. W3110
MNQQLSWRTIVGYSLGDVANNFAFAMGALFLLSYYTDVAGVGAAAAGTMLLLVRVFDAFADVFAGRVVDSVNTRWGKFRPFLLFGTAPLMIFSVLVFWVLTDWSHGSKVVYAYLTYMGLGLCYSLVNIPYGSLATAMTQQPQSRARLGAARGIAASLTFVCLAFLIGPSIKNSSPEEMVSVYHFWTIVLAIAGMVLYFICFKSTRENVVRIVAQPSLNISLQTLKRNRPLFMLCIGALCVLISTFAVSASSLFYVRYVLNDTGLFTVLVLVQNLVGTVASAPLVPGMVARIGKKNTFLIGALLGTCGYLLFFWVSVWSLPVALVALAIASIGQGVTMTVMWALEADTVEYGEYLTGVRIEGLTYSLFSFTRKCGQAIGGSIPAFILGLSGYIANQVQTPEVIMGIRTSIALVPCGFMLLAFVIIWFYPLTDKKFKEIVVEIDNRKKVQQQLISDITN

SEQ ID NO: 113 - protein, 427 aa, Escherichia coli str. K-12 substr. W3110
MDFQLYSLGAALVFHEIFFPESSTAMALILAMGTYGAGYVARIVGAFIFGKMGDRIGRKKVLFITITMMGICTTLIGVLPTYAQIGVFAPILLVTLRIIQGLGAGAEISGAGTMLAEYAPKGKRGIISSFVAMGTNCGTLSATAIWAFMFFILSKEELLAWGWRIPFLASVVVMVFAIWLRMNLKESPVFEKVNDSNQPTAKPAPAGSMFQSKSFWLATGLRFGQAGNSGLIQTFLAGYLVQTLLFNKAIPTDALMISSILGFMTIPFLGWLSDKIGRRIPYIIMNTSAIVLAWPMLSIIVDKSYAPSTIMVALIVIHNCAVLGLFALENITMAEMFGCKNRFTRMAISKEIGGLIASGFGPILAGIFCTMTESWYPIAIMIMAYSVIGLISALKMPEVKDRDLSALEDAAEDQPRVVRAAQPSRSL

SEQ ID NO: 114 - protein, 440 aa, Escherichia coli str. K-12 substr. W3110
MQATATTLDHEQEYTPINSRNKVLVASLIGTAIEFFDFYIYATAAVIVFPHIFFPQGDPTAATLQSLATFAIAFVARPIGSAVFGHFGDRVGRKATLVASLLTMGISTVVIGLLPGYATIGIFAPLLLALARFGQGLGLGGEWGGAALLATENAPPRKRALYGSFPQLGAPIGFFFANGTFLLLSWLLTDEQFMSWGWRVPFIFSAVLVIIGLYVRVSLHESPVFEKVAKAKKQVKIPLGTLLTKHVRVTVLGTFIMLATYTLFYIMTVYSMTFSTAAAPVGLGLPRNEVLWMLMMAVIGFGVMVPVAGLLADAFGRRKSMVIITTLIILFALFAFNPLLGSGNPILVFAFLLLGLSLMGLTFGPMGALLPELFPTEVRYTGASFSYNVASILGASVAPYIAAWLQTNYGLGAVGLYLAAMAGLTLIALLLTHETRHQSL

SEQ ID NO: 115 - protein, 460 aa, Escherichia coli str. K-12 substr. W3110
MKSEVLSVKEKIGYGMGDAASHIIFDNVMLYMMFFYTDIFGIPAGFVGTMFLVARALDAISDPCMGLLADRTRSRWGKFRPWVLFGALPFGIVCVLAYSTPDLSMNGKMIYAAITYTLLTLLYTVVNIPYCALGGVITNDPTQRISLQSWRFVLATAGGMLSTVLMMPLVNLIGGDNKPLGFQGGIAVLSVVAFMMLAFCFFTTKERVEAPPTTTSMREDLRDIWQNDQWRIVGLLTIFNILAVCVRGGAMMYYVTWILGTPEVFVAFLTTYCVGNLIGSALAKPLTDWKCKVTIFWWTNALLAVISLAMFFVPMQASITMFVFIFVIGVLHQLVTPIQWVMMSDTVDYGEWCNGKRLTGISFAGTLFVLKLGLAFGGALIGWMLAYGGYDAAEKAQNSATISIIIALFTIVPAICYLLSAIIAKRYYSLTTHNLKTVMEQLAQGKRRCQQQFTSQEVQN

SEQ ID NO: 116 - protein, 420 aa, Francisella tularensis subsp. tularensis SCHU S4
MNNHIKAQILLLCFCQLIIIAAMEMSNPFLPIYLQSLGDFDLLPVNAWNVLAYAMPLISAMLFSPFWGKYADRFGYKTMILRAALALAIIQLLIYLTNSALLFITLRFIQGAIAGFLLAAQSYAVVIVSKEFRSRILAWLQSSTAIGIAIGPLIGGVLATLLSYNQIFLICAVIACCIFIILLIKLESISNPSLLSKQDNNKNCYHKNTSNNYLIYVYFSVIFLAILLSQTAKFLPQSFFAIYAKEFFSSDPIIIAAIYAAPAFSLLLFSAPIGYLFDKLLNKQVNNQYFFASSYFIILFAISTVAIYVHGFTHNIYLILAARFILGVTFAGTLPCLFSLACRTKDNNFGFLIGYCNTFSKFGNLCGIFLGGFIFQISSMQTVFYVTAIIYMFLVVIYICLLYLFDLKSSNFKNKELINV

SEQ ID NO: 117 - protein, 412 aa, Kluyvera ascorbata
MNRQSWLLNLSLLKTHPAFRAVFIARFISILSLGLLGVAIPVQIQMMTHSTWQVGLSVTLTGGAMFVGLMVGGVLADRYERRKLILLARGTCGVGFIGLWLNAMLPEPSLVAIYLLGLWDGFFASLGVTALLAATPALVGRENLMQAGAITMLTVRLGSVISPMVGGLLLASGGVSWNYLLAALGTFITTLTLLRLPQLPPPPQPREHPFTSLVAGFRFLLSSPLIGGIALLGGLVTMASAVRVLYPALAQGWQMPAASIGLLYAAIPLGAAFGALTSGRLAQSERPGWVMLLATVGAFVAIGLFAMMPHWALGMVCLALFGWLSAISSLLQYTLIQTQTPEAMLGRINGLWTAQNVTGDAIGAALLGGLGAMLTPVASASVSGWGLAVVGGILLLVLGELRRFRREIPESA

SEQ ID NO: 118 - protein, 418 aa, Kluyvera intermedia strain CAV1151
MKKQSRLLNVSLLKTHPAFRAVFLARFISILSLGLLGVAVPVQIQAMTGSSWQVGLSVTLTGGAMFVGLMTGGVLADRYERKKLILLARGTCGIGFIGLWINALLPEPSLLAIYLLGLWDGFFASLGVTALLAATPALVGRENLMQAGAITMLTVRLGSVISPMCGGLLLATGGVAWNYGLAAAGTFITLLPLLSLPALPSPPQPREHPFSSLVAGIRFLFNSPLIGGIALLGALLTMASAVRVLYPALAVEWHISAAQIGVLYAAIPFGAATGALTSGRLAQHPRPGLLMLCCSVGAFVAIGLFSLMPYWVLGAAFLALFGWLSAISSLLQYTLIQTQTPEAMLGRINGLWTAQNVTGDATGAAVLGGLGAVMTPATSASSSGFVLALVGVALVLMLSELRRFRQTPVASPDGGAGA

SEQ ID NO: 119 - protein, 411 aa, Leclercia adecarboxylata strain USDA-ARS-USMARC-60222
MTRQSWLLNLSLLKTHPAFRAVFIARFISILSLGLLGVAVPVQIQAMTHSSWLVGLSVTLTGGAMFIGLMVGGVLADRYERKKLILLARGTCGVGFVGLCLNAMLPEPSLIAIYLLGLWDGFFASLGVTALLAATPALVGRENLMQAGAITMLTVRLGSVISPMVGGLLLGTGNVAWNYGLAAAGTFITTLTLLRLPLLPPPPQPREHPLKSLLAAIRFLFSNPLIGGIALLGGLLTMASAVRVLYPALAMEWQMSVSQIGLLYAAIPLGAAFGALTSGNLAQSARPGLIMLLATLASFIAIGFFSLMPVWALGVICLALFGWLSAVSSLLQYTLIQTQTPEAMLGRINGLWTAQNVTGDAIGAAILGGLGVIMTPVASASSSGFVLAIVGAMLLVVLAELRRFRQTPATV

SEQ ID NO: 120 - protein, 397 aa, Staphylococcus aureus subsp. aureus NCTC 8325
MTKYFFSSSFLLFLGNWIGQIGLNWFVLTTYHNAVYLGIVNFCRLVPILLLSVWAGAIADKYDKGRLLRITISSSFLVTAILCVLTYSFTAIPISVIIIYATLRGILSAVETPLRQAILPDLSDKISTTQAVSFHSFIINICRSIGPAIAGVILAVYHAPTTFLAQAICYFIAVLLCLPLHFKVTKIPEDASRYMPLKVIIDYFKLHMEGRQIFITSLLIMATGFSYTTLLPVLTNKVFPGKSEIFGIAMTMCAIGGIIATLVLPKVLKYIGMVNMYYLSSFLFGIALLGVVFHNIVIMFICITLIGLFSQWARTTNRVYFQNNVKDYERGKVLSIIMMDRGMIPLGSLLMSICADVFGIVRTFSIMGISTICITMVFYFINRKLKLKLEESNHGIS

SEQ ID NO: 121 - protein, 414 aa, Salmonella enterica subsp. arizonae
MNRQSWLLNLSLLKTHPAFRAVFLARFISIVSLGLLGVAVPVQIQMMTHSTWQVGLSVTLTGGAMFIGLMVGGVLADRYERKKVILLARGTCGIGFIGLCVNVLLPEPSLLAIYLLGLWDGFFASLGVTALLAATPALVGRENLMQAGAITMLTVRLGSVISPMLGGILLASGGVAWNYGLAAAGTFITLLPLLTLPRLPVPPQPRENPFIALLAAFRFLLASPLIGGIALLGGLVTMASAVRVLYPALAMSWQMSAAQIGLLYAAIPLGAAIGALTSGQLAHSVRPGLIMLVSTVGSFLAVGLFAIMPVWIAGVICLALFGWLSAISSLLQYTLLQTQTPENMLGRMNGLWTAQNVTGDAIGAALLGGLGAMMTPVASASVSGFGLVIIGLLLLLVLGELRRFRQTPPVSDAG

SEQ ID NO: 122 - protein, 414 aa, Salmonella enterica subsp. enterica serovar Derby
MNRQSWLLNLSLLKTHPAFRAVFLARVISIVSLGLLGVAVPVQIQMMTHSTWQVGLSVTLTGGAMFIGLMVGGVLADRYERKKVILLARGTCGIGFIGLCVNALLPEPSLLAIYLLGLWDGFFASLGVTALLAATPALVGRENLMQAGAITMLTVRLGSVISPMLGGILLASGGVAWNYGLAAAGTFITLLPLLTLPRLPVPPQPRENPFIALLATFRFLLASPLIGGIALLGGLVTMASAVRVLYPALAMSWQMSAAQIGLLYAAIPLGAAIGALTSGQLAHSVRPGLIMLVSTVGSFLAVGLFAIMPVWIAGVICLALFGWLSAISSLLQYTLLQTQTPENMLGRMNGLWTAQNVTGDAIGAALLGGLGAMMTPVASASVSGFGLVIIGLLLLLVLGELRRFRQTPPVSDAG